(19) United States
(12) Patent Application Publication
(10) Pub. No.: US 2013/0177629 A1
(43) Pub. Date: Jul. 11, 2013

(54) LIPOSOMAL COMPOSITIONS

(71) Applicant: Nuvo Research GmbH, Leipzip (DE)

(72) Inventors: Rainer Martin, Waghaeusel (DE); Jürgen Arnhold, Leipzig (DE); Robert Seifert, Leipzip (DE); Dominic King-Smith, San Diego, CA (US); Tejas Desai, Mississauga (CA); Andreas Wagner, Vienna (AT)

(73) Assignee: Nuvo Research GmbH, Leipzip (DE)

(21) Appl. No.: 13/724,963

(22) Filed: Dec. 21, 2012

Related U.S. Application Data
(60) Provisional application No. 61/579,326, filed on Dec. 22, 2011.

(51) Int. Cl. A61K 9/127

(52) U.S. Cl. CPC A61K 9/127
USPC 424/450; 424/661; 424/662

(57) ABSTRACT

The present application includes liposomes and liposomal compositions that comprise chlorite, chlorate or a mixture thereof entrapped inside the liposome core, methods for their preparation and methods of use, in particular as medicaments.

The present application claims the benefit of priority of co-pending U.S. provisional patent application No. 61/579,326 filed on Dec. 22, 2011, the contents of which are incorporated herein by reference in their entirety.

### FIELD OF THE APPLICATION

The present application relates to liposomal compositions comprising chlorite, chlorate, or a mixture thereof, methods for their preparation and their use, for example, as medicaments.

### BACKGROUND OF THE APPLICATION

### Liposomes

Liposomes have been extensively studied as vehicles for drug delivery. There have been reports of the effective use of liposomes for the delivery of drugs and vaccines (Gregoriadis, G. TIBTECH, 1995, 13:527-537, and referenced cited therein). For example, liposome-based drug delivery systems are widely used for intravenous anticancer chemotherapy for administering drugs such as doxorubicin HCl which is marketed as Doxil and Myocet (Abraham et al. Methods Enzymol. 2005, 391:71-97). Liposomes can also be used to deliver inhaled aerosol drugs, for example: i) insulin (Huang et al. J Control Release, 2006, 113:9-14); ii) antibiotics, particularly targeting tuberculosis infection in alveolar macrophages (Vyas et al. Int J Pharm. 2005, 296:12-25); iii) anticancer chemotherapy drugs (Verschraegen et al. Clin Cancer Res. 2004, 10:2319-2326); and iv) others (see U.S. Pat. No. 5,049,388).

It is more common for organic molecules of a certain size to be incorporated into liposomes. Smaller molecules, such as ethanol, glucose, ammonia and acetate are known to permeate through the lipid bilayers of the liposome. This behavior is known as leakage.

While there are examples of the successful use of liposomes as drug-delivery systems, there are still challenges to be met for many applications. Chemical and physical stability, clearance, entrapment efficiency and targeting are some of the largest challenges, in particular when smaller molecules are to be entrapped.

### Chlorite, Chlorate and Mixtures Thereof

The chlorite ion, ClO₂⁻, referred to herein as chlorite, has been used in various contexts. Sodium chlorite is a strong oxidizing agent, and has been used in water purification, disinfection, and in bleaching and deodorizing. Under acidic conditions, sodium chlorite produces highly toxic chlorine dioxide gas therefore aqueous solutions employed are usually provided as extremely basic (approximately pH 13) solutions, with the pH adjusted using a basic agent such as sodium hydroxide.

Chlorate salts have also been used in various contexts. The chlorate ion, ClO₃⁻, referred to herein as chlorate, is a strong oxidizing agent, and has been used in pyrotechnics, disinfection and pesticides. In addition, chlorate has been used as an antifungal agent for the treatment of skin diseases (U.S. Pat. No. 5,427,801) and has been found to reversibly inhibit proteoglycan sulfation in Chlamydia trachomatis-infected cells (J Med Microbiol February 2004 vol. 53 no. 2 93-95).

Compositions comprising a mixture of chlorite and chlorate have also been used in various contexts, for example, to treat various diseases or conditions. Stabilized chlorite is a non-limiting example of such compositions. For example, U.S. Pat. Nos. 4,725,437, 4,507,285 and 4,851,222 disclose the use of a stabilized chlorite to treat wounds and infections and to cause regeneration of bone marrow. The use of a stabilized chlorite to inhibit antigen specific immune responses is described in U.S. patent application publication no. 2011/0076344. PCT patent application publication no. 2001/017030 discloses the treatment of a wide range of macrophage-related disorders through the administration of an oxidative agent, such as a stabilized chlorite. U.S. Pat. No. 7,105,183 describes the use of a stabilized chlorite for treating macrophage-associated neurogenerative diseases. U.S. Pat. No. 5,877,222 describes the use of a stabilized chlorite to treat AIDS-related dementia. PCT patent application publication no. 2008/145376 reports the use of a stabilized chlorite to treat allergic asthma, allergic rhinitis and atopic dermatitis. PCT patent application publication no. 2007/009245 discloses the use of a stabilized chlorite in combination with fluoropyrimidines for cancer treatment. PCT patent application publication no. 2001/012205 discloses the use of a stabilized chlorite to treat cancer and other conditions that are affected by modulating macrophage function. EP patent application no. 255145 discloses the use of a stabilized chlorite in ophthalmology. A phase II study has been reported that evaluates the use of a stabilized chlorite in the treatment of patients with late hemorrhagic radiation cystitis and proctitis (Veerasarn, V. et al. Gynecologic Oncology, 2006, 100:179-184). Stabilized chlorite has been shown to prolong xenograft survival in a rodent cardiac model (Hansen, A. et al. Pharmacology & Toxicology, 2001, 89:92-95) and in other studies (Kemp, E. et al. Transplantation Proceedings, 2000, 32:1018-1019). The antimicrobial effects of a stabilized chlorite have also been reported (Stoll, P. et al. Zeitschrift fuer Antimikrobielle and Antineoplastische Chemotherapie, 1993, 11:15-20; Stoll, P. et al. Chemotherapy, 1993, 39:40-47; Hollfelder, K. et al. Reakt. Sauerstoffspezies Med. 1987, 253-262; Adamczyk, R. et al. Reakt. Sauerstoffspezies Med. 1987, 247-252; Gillissen, G. et al. Arzneimittel-Forschung, 1986, 36:1778-1782; Ullmann, U. et al. Infection, 1985, 13:S236-S240; Ullmann, U. et al. Infection, 1984, 12:225-229). A recent study showed that a stabilized chlorite is able to stimulate natural killer (NK) cell cytotoxicity against malignant cells which relates to the ability of stabilized chlorite to enhance immunity against tumor cells (Kühne, L. et al. J. Biomedicine and Biotechnology, 2011, p. 436587).

In the above publications, the chlorite is in the form of commercially available formulations of stabilized chlorite known as Oxovasin™ (a topical formulation) or WF10 (a drug product for intravenous administration). Oxovasin™ and WF10 are dilutions of OXO-K993, itself an aqueous solution. Originally OXO-K993 was thought to be a solution comprising a compound called tetrachlorodecaoxygen (TCDO) as the active ingredient and the terms tetrachlorodecaoxygen, tetrachlorodecaoxide and TCDO are frequently used in older literature to refer to the active in WF10 and Oxoferin. However, subsequent analytical testing determined that OXO-K993 does not contain TCDO, but rather that it is an aqueous solution of chlorite, chloride, chlorate, and sulfate ions, with sodium as the cation. Therefore intravenous administration of WF10 or topical application of Oxoferin™ to a patient entails delivery of a mixture of ions. OXO-K993 is available from Nuvo Manufacturing GmbH (Wanzleben, Germany). OXO-K993 and its preparation are described in U.S. Pat. No. 4,507,285. Review articles describing OXO-K993 and WF10 have been published which describes the various uses of this substance known at the time (Drugs in R&D, 2004, 5:242-244; McGrath et al. Current Opinion in Investigational Drugs, 2002, 3:365-373).

### Encapsulation Methods

U.S. Pat. No. 5,269,979 discloses a method of forming vehicles called solvent dilution microcarriers (SDMCs) for encapsulating passenger molecules. The encapsulating vehicles are formed using a multistep method that first involves preparation of a “formed solution”, followed by an organization step which results in the creation of the SDMCs from the “formed solution”. The “formed solution” is prepared by dissolving an amphiphatic material and a passenger molecule in an organic solvent, followed by addition of water to obtain a turbid solution and then addition of more organic solvent to obtain the clear “formed solution”. The formed solution may be used immediately or stored. SDMC's are prepared using an organization step that may comprise diluting the “formed solution” into an aqueous system, aerosolization, or rehydration in situ. In the SDMCs, the passenger molecule is entrapped in the bilayer itself, or in association with a component of the bilayer, rather than inside the space created by a spherical bilayer. Among the carrier molecules that were encapsulated in an SDMC was tetrachlorodecaoxide (TCDO). It is of note that the process described in this application does not involve a “purification” step so that all materials used in the preparation of the SDMC's remain in the final solution used for testing.

Canadian patent application no. 2,636,812 discloses an enveloping membrane for discharging an enclosed agent in an aqueous medium. In one example, the enclosed agent is an oxidizing chlorine-oxygen compound, in particular, TCDO. The enveloping membrane is insoluble and water-permeable in a neutral aqueous medium and is generally made from cationic and/or anionic water-insoluble polymers. The compositions prepared in this application are useful for the disinfection and purification of liquids and of substrate surfaces and for the disinfection of water.

Korean patent application publication no. KR2003/072766 discloses oral hygiene compositions comprising chlorite, specifically sodium chlorite, and zinc ions encapsulated in a liposome. The composition can additionally contain carriers or excipients, can have a double or single phase structure and a pH of 7-8.5. In an example, the liposome is prepared from lecithin.

PCT patent application publication no. WO 00/19981 discloses antimicrobial preparations which include chlorite in combination with a peroxy compound (e.g., hydrogen peroxide), and methods for using these preparations for disinfection of articles or surfaces. The preparations can be formulated as liposomes and have a pH of between 6.8-7.8. Both the chlorite and peroxy compound are required for microbial activity. Notably, no actual liposomal formulations were prepared in this application.

U.S. patent application publication no. 2007/0145328 discloses chlorite, such as sodium chlorite, formulations for parenteral, systemic or intravenous administration comprising chlorite and a pH adjusting agent. The pH adjusting agent adjusts the pH of the formulations so that it is in the range of about 7 to about 11.5. The formulations are taught to be less toxic than WF10.

Panasenko et al. Membr Cell Biol. 1997; 11(2):253-8 disclose the ability of sodium hypochlorite (NaClO), chlorite (NaClO₂), chlorate (NaClO₃) and perchlorate (NaClO₄) to initiate lipid peroxidation. The liposomes are prepared from egg phosphatidylcholine at a pH of 7.4 in simple NaCl and buffer. The oxochloric acid salts are only added to the outer phase. No steps are taken to load the vesicles with chlorite or chlorate or to remove the oxochloric acid salts from the external phase.

U.S. patent application publication no. 2011/0052655 describes the encapsulation of biocides in micro- or nano-capsules (including liposomes) for controlling protozoa trophozoites and cysts in aqueous systems.

U.S. patent application publication no. 2011/0177147 describes the encapsulation of an antimicrobial composition in combination with at least one stabilizer for removing bio-fouling in industrial water bearing systems. The antimicrobial composition can be a non-oxidizing biocidal compound, such as isothiazolin, and the stabilizer can be a buffer that contains chlorate.

### SUMMARY OF THE APPLICATION

The present application is directed to liposomal compositions comprising chlorite, chlorate or a mixture thereof and methods for their preparation and use.

Accordingly, the present application includes a liposomal composition comprising liposomes having at least one lipid bilayer and chlorite, chlorate or a mixture thereof encapsulated inside the liposomes, wherein the lipid bilayer is comprised of one or more suitable lipids. Typically, there is a plurality of separated liposomes (also referred to as vesicles) in the composition. Typically the vesicles are dispersed in an aqueous phase and encapsulate one or more aqueous compositions. Therefore, in accordance with one embodiment, the chlorite, chlorate or a mixture thereof are encapsulated in a plurality of vesicles the walls of which comprise one or more lipid bilayers. The present application also includes a liposome comprising at least one lipid bilayer and chlorite or chlorate or a mixture thereof entrapped inside the liposomes, wherein the lipid bilayer is comprised of one or more suitable lipids.

The present application further includes a liposomal composition comprising encapsulated and non-encapsulated chlorite, chlorate or a mixture thereof, wherein the encapsulated chlorite, chlorate, or a mixture thereof is in the internal phase and the non-encapsulated chlorite, chlorate, or a mixture thereof is in the external phase of the composition. Alternately, the external phase may comprise a substance other than chlorite, chlorate or a mixture thereof, such as another therapeutic agent or sodium chloride. In one embodiment, the pH of the internal phase and external phase is the same. In another embodiment, the pH of the internal phase and external phase are different, for example, the pH in the inner phase may be pH of about 5 to about 14, about 6 to about 13, about 8 to about 12.5, or about 10 to about 12, and the pH of the external phase may be approximately neutral such as about 6-8. In one embodiment of the instant invention the pH of the inner and external phases is adjusted by means of buffers such as, for example, a carbonate buffer.

In a further embodiment, the liposome is comprised of lipids that are suitable for the entrapment of chlorite, chlorate or a mixture thereof having a pH of about 5 to about 14, about 8 to about 13, about 9 to about 12.5, or about 10 to about 12. In an embodiment, the lipids are selected from those that form liposomes that are impermeable to leakage of chlorite and/or chlorate ions at about 5° C. or above. Such lipids include, for example, phospholipids such as phosphatidylcholines having saturated and/or unsaturated fatty acid chains of a sufficient length and/or sphingolipids such as sphingomyelin or appropriate mixtures of such lipids showing the desired behavior.

In another embodiment, the chlorite is a stabilized chlorite composition, such as OXO-K993 or a composition comprising 1-10%, 10-20%, 20-30%, 30-50% or 50-90% (w/v) OXO-K993. In a further embodiment, the stabilized chlorite comprises 10% (w/v) OXO-K993 (OXO-K993 diluted in this manner is known as WF10).

In an embodiment, the stabilized chlorite comprises 2% (w/v) OXO-K993. In a further embodiment, the stabilized chlorite comprises about 2% (w/v) OXO-K993, about 2% (w/v) glycerol and about 96% (w/v) water. Such a composition is sold commercially as Oxovasin™ or Oxoferin™ (Nuvo Manufacturing GmbH, Wanzleben, Germany).

In a further embodiment, the liposomal composition comprises encapsulated chlorite present in an amount of about 0.01% (w/w) to about 50% (w/w), about 0.1% (w/w) to about 20% (w/w) or about 0.5% (w/w) to about 10% (w/w) of the total encapsulated ion content of the composition.

In yet a further embodiment, the liposomal composition comprises encapsulated chlorate present in an amount of about 0.01% (w/w) to about 50% (w/w), about 0.1% (w/w) to about 20% (w/w) or about 0.5% (w/w) to about 10% (w/w) of the total encapsulated ion content of the composition.

In still a further embodiment, the liposomal composition comprises an encapsulated mixture of chlorate and chlorite ions present in an amount of about 0.01% (w/w) to about 50% (w/w), about 0.1% (w/w) to about 20% (w/w) or about 0.5% (w/w) to about 10% (w/w) of the total encapsulated ion content of the composition. Any composition of matter containing chlorite and/or chlorate ions may also have at least one counter ion to maintain charge neutrality. Thus, according to one embodiment of the present application, the liposomal compositions comprise one or more cations. Non-limiting examples of possible cations include alkali metal cations (such as sodium or Na⁺) and alkaline earth cations. In another embodiment, the liposomal compositions comprising chlorite ions, chlorate ions or a mixture thereof, further comprise sodium and/or potassium counter ions. In a further embodiment, the liposomal compositions are isotonic with respect to a subject's body fluids. Determining isotonicity in this respect is well within the knowledge of the skilled artisan.

The present application also includes a method of preparing liposomes having at least one lipid bilayer and chlorite, chlorate or a mixture thereof encapsulated inside the liposomes, wherein the lipid bilayer is comprised of one or more suitable lipids that could be from natural, semi-synthetic or synthetic source. In one embodiment the method comprises:

(a) adding an aqueous solution of chlorite, chlorate or a mixture thereof to a vessel having a film of the one or more lipids on at least a portion of an inner surface;
(b) agitating the vessel under conditions sufficient to wholly or partially remove the film from the inner surface to provide a turbid solution comprising the chlorite- and/or chlorate-entrapped liposomes;
(c) treating the turbid solution to reduce the average diameter of the liposomes a desired amount; and
(d) optionally treating the liposomes to remove chlorite, chlorate or a mixture thereof from a solution external to the liposomes.

Following the methods of the application, it is an embodiment that the inclusion rate of chlorite, chlorate or a mixture thereof in the inventive liposomes is from about 0.1% to about 50%, about 0.5% to about 25%, about 1% to about 15%, about 1% to about 10%, or about 1% to about 5%.

In another embodiment, the liposomes of the present application are prepared using an ethanol injection method. In a further embodiment, the liposomes are prepared using an ethanol injection method comprising the crossflow technique.

The present application also includes pharmaceutical compositions comprising liposomes with at least one physiologically acceptable carrier or excipient.

The application also includes a use of the compositions of the present application as medicaments. The compositions of the application may be sterile or non-sterile depending on their intended use. The compositions of the invention may also be pyrogen-free.

The application further includes a method for treating a disease, disorder or condition for which administration of chlorite, chlorate or a mixture thereof is beneficial comprising administering an effective amount of a composition of the application to a subject in need thereof.

The application further includes a use of a composition of the application for treating a disease, disorder or condition for which administration of chlorite, chlorate or a mixture thereof is beneficial.

In one embodiment, a method of using the composition comprises informing a user of certain safety or clinical effects. For example, the user may be informed that liposomal compositions are more stable, target specific, or therapeutically effective than non-liposomal compositions that provide, or would be expected to provide, a similar therapeutic effect. The user may also be informed that the composition is in one or more respects safer than non-liposomal formulations that provide, or would be expected to provide, similar therapeutic effect. For instance, treatment with the liposomal compositions may result in lowered side effects, such as, reduced vein irritation (phlebitis), thereby increasing patient tolerance and compliance. The user may additionally be informed that liposomal compositions may be administered at a faster rate (e.g IV push vs. dilution infusion) or at a reduced volume compared to non-liposomal formulations that provide, or would be expected to provide, similar therapeutic effect. The user may also be informed that the liposomal compositions provide an extended, controlled or delayed release of the active ingredient. Further, the user many be informed that this extended or delayed release may be customized or tuned, depending on the identity of the lipids in the liposome and the timing required for therapeutic treatment by the user. The user may be informed by way of published material such as a label or product insert.

Other features and advantages of the present application will become apparent from the following detailed description. It should be understood, however, that the detailed description and the specific examples, while indicating embodiments of the application, are given by way of illustration only, since various changes and modifications within the spirit and scope of the application will become apparent to those skilled in the art from this detailed description.

### BRIEF DESCRIPTION OF THE DRAWINGS

The embodiments of the application will now be described in greater detail with reference to the attached drawings in which:

FIG. 1 shows a WF10 dilution curve used to relate the measured absorbance of chlorite/chlorate to a certain inverse dilution factor in the o-toludin Method B of Example 7 for the detection of chlorite/chlorate in a sample. The inverse dilution factor is plotted versus the measured absorbance at 447 nm. The left hand plot shows the whole dataset, while the right hand plot presents only the non-linear domain. The top and bottom lines represent the fits on the linear and non-linear parts of the dataset, respectively. The first intersection of both marks the transition between the two domains.

FIG. 2 is a graph showing leakage of chlorite and chlorate from liposomes prepared from 1-palmitoyl-2-oleoyl-sn-3-glycero-3-phosphocholine (POPC) and stored at 5° C., 23° C. and 37° C.

FIG. 3 is a graph showing leakage of chlorite and chlorate from liposomes prepared from sphingomyelin (SM) and stored at 5° C., 23° C. and 37° C. The cluster of data points at the end of the graph at approximately 522 h is a measurement after the temperature was purposefully increased to 37° C.

FIG. 4 is a graph showing the leakage of chlorite and chlorate from liposomes prepared from POPC:POPG (1-palmitoyl-2-oleoyl-sn-glycero-3-phospho-(1′-rac-glycerol) at a ratio of 3:1 and stored at 5° C., 23° C. and 37° C.

FIG. 5 is a graph showing the leakage of chlorite and chlorate from liposomes prepared from POPC:POPG at a ratio of 2:1 and stored at 5° C., 23° C. and 37° C.

FIG. 6 is a graph showing the leakage of chlorite and chlorate from liposomes prepared from POPC:POPG at a ratio of 1:1 and stored at 5° C., 23° C. and 37° C.

FIG. 7 is a graph showing the leakage of chlorite and chlorate from liposomes prepared from 1,2-dimyristoyl-sn-glycero-3-phosphocholine

(DMPC) and stored at 5° C. and 23° C. for 1, 5 and 7 days.

FIG. 8 is a graph showing the leakage of chlorite and chlorate from liposomes prepared from sphingomyelin SM. Storage was at 5° C. (blue) and 23° C. (red) followed by a temperature increase to 37° C. which led to immediate leakage.

FIG. 9 is a graph showing the leakage of chlorite and chlorate from liposomes prepared from DPPC and DPPC/DMPC and stored at 22° C. and 38° C.

FIG. 10 presents an overview of the long term stability studies on DPPC and DPPC/DMPC liposomes. Each bar represents the outcome of the leakage experiments of a sample at room (ca. 22° C.) and fridge (ca. 6° C.) temperature. The x-axis represents the time in days. The medium grey colour marks liposomes where the WF10 content in the outer medium was found to be below the LOD. The lighter grey colour marks periods where the WF10 content in the outer medium was below the LOQ. The darkest grey colour indicates WF10 content above the LOQ.

FIG. 11 shows the results of the High Resolution Leakage (HRL) experiment performed with SX122 on Dec. 12, 2011, described in Example 12. The graphs for both samples can be seen. The temperature curves refer to the right hand y-axis. The amount of leaked substance is provided as a fraction of the total sample volume. The experiment was used to determine the temperature at which leakage starts within a few hours.

FIG. 12 shows the results of the HRL Experiment performed with SX122 on Jan. 6, 2012, described in Example 12. The graphs for both samples can be seen. The temperature curves refer to the right hand y-axis. The amount of leaked substance is provided as a fraction of the total sample volume. The experiment examines the leakage behavior at 38° C. The experiment runs for several days, indicating a very slow leakage at this temperature.

FIG. 13 shows the results of the HRL Experiment performed with SX126 on Jan. 26, 2012, described in Example 12. The graphs for both samples can be seen. The temperature curves refer to the right hand y-axis. The amount of leaked substance is provided as a fraction of the total sample volume. Partial leakage can be seen in the graph.

FIG. 14 shows the results of the HRL Experiment performed with SX126 on Feb. 22, 2012, described in Example 12. The graphs for both samples can be seen. The temperature curves refer to the right hand y-axis. The amount of leaked substance is provided as a fraction of the total sample volume.

FIG. 15 shows the results of the HRL Experiment performed with SX126 on Jan. 25, 2012, described in Example 12. The graphs for both samples can be seen. The temperature curves refer to the right hand y-axis. The amount of leaked substance is provided as a fraction of the total sample volume.

FIG. 16 is a graph showing the amount of released substance as a fraction of the total sample volume, plotted versus the time for experiments reported in Example 14. The large graph shows the whole experiment, as well as the upper leakage limit, where the enclosed WF10 would have been completely released. This is at approximately 7.5% of the whole sample volume, showing, therefore, also the encapsulation capacity of the liposomes. The smaller graph covers in more detail the time period where some leakage took place.

FIG. 17 shows a schematic of the preparation of liposomes using the ethanol injection method in one embodiment of the present application. Liposomes exiting the steel chamber encapsulate WF10 and are dispersed in an outer phase comprising WF10.

FIG. 18 is a graph showing the amount of chlorite collected in the filtrate during the diafiltration process to prepare WF10 containing liposomes using the ethanol injection method.

FIG. 19 is a graph showing the amount of chlorite collected in the filtrate during the diafiltration process to prepare WF10 containing liposomes using the ethanol injection method (repeat of the experiments reported in FIG. 18).

FIG. 20 is a graph showing the amount of chlorite collected in the filtrate during the diafiltration process to prepare WF10 containing liposomes using the ethanol injection method. The liposomes were prepared using double the concentration of WF10 compared to the experiments reported in FIGS. 18 and 19.

FIG. 21 is a graph showing collagen induced arthritis (CIA) score of mice receiving WF10, WF10 in liposomal form or NaCl (i.p.) after the induction of CIA.

FIG. 22 is a bar graph showing CIA score on Day 16 for mice receiving WF10, WF10 in liposomal form or NaCl (i.p.).

### DETAILED DESCRIPTION OF THE APPLICATION

### I. Definitions

Unless otherwise indicated, the definitions and embodiments described in this and other sections are intended to be applicable to all embodiments and aspects of the application herein described for which they are suitable as would be understood by a person skilled in the art.

The terms “a,” “an,” or “the” as used herein not only include aspects with one member, but also includes aspects with more than one member. For example, an embodiment including “a phospholipid” should be understood to present certain aspects with one phospholipid or two or more additional phospholipids.

In compositions comprising an “additional” or “second” component, the second component as used herein is chemically different from the other components or first component. A “third” component is different from the other, first, and second components, and further enumerated or “additional” components are similarly different.

The term “agent” as used herein indicates a compound or mixture of compounds that, when added to a composition, tend to produce a particular effect on the composition's properties.

The term “chlorite” as used herein refers to the anion “ClO₂⁻”.

Anionic species typically exist in aqueous solutions in dissociated form, however the anion is often derived from a parent salt containing an anion and a cation.

The term “chlorate” as used herein refers to the anion “ClO₃⁻”. Anionic species typically exist in aqueous solutions in dissociated form, however the anion is often derived from a parent salt containing an anion and a cation.

The term “stabilized chlorite” as used herein refers to a composition or substance, comprising chlorite ions (ClO₂⁻) and in which the concentration of chlorite ions, the pH and/or the activity remains stable for an acceptable period of time prior to use. In a stabilized chlorite, the chlorite ions do not substantially degrade and the activity of the chlorite ions is substantially maintained prior to use. The stabilized chlorite may contain a buffer, such as a sodium carbonate/sodium hydroxide buffer system, which maintains the alkaline pH of the formulation. The concentration of chlorite ions may be monitored, for example, by high performance liquid chromatography (HPLC).

“WF10” as used herein refers to a 10% (w/v) aqueous dilution of the drug substance OXO-K993 analytically characterized as a solution containing the ions chlorite 4.25%, chlorate (1.5%), chloride (2.0%), sulfate (0.7%) and sodium (4.0%).

The term “an acceptable period of time” as used herein means at least about 1 day, at least about 1 week, at least about 30 days, at least about six months, at least about one year, at least about two years, or at least about the time between preparation and use.

The term “liposome” as used herein refers to artificially prepared vesicles composed of at least one lipid bilayer surrounding an inner core. The inner phase, internal phase or inner core (used interchangeably herein) contains substances, such as chlorite, chlorate or a mixture thereof. The vesicle may be used to deliver the substances, for example, topically or within the body. There are three types of liposomes—MLV (multilamellar vesicles), SUV (small unilamellar vesicles—25-300 nm in diameter) and LUV (large unilamellar vesicles—>300 nm in diameter). The volume of material exterior to the vesicles may be referred to as the external phase, outer phase or continuous phase. These terms are used interchangeably herein. A liposomal composition will comprise a plurality of individual, separate liposomes and the inner and outer phase usually comprise water.

Sphingolipids are a class of lipids containing as a backbone sphingosine (2-amino-4-octadecene-1,3-diol) attached to a variety of head groups.

Sphingomyelin (SM) refers to a type of sphingolipid found in animal cell membranes, especially in the membranous myelin sheath that surrounds some nerve cell axons. Sphingomyelin has a ceramide core (sphingosine bonded to a fatty acid via an amide linkage) and a polar head group which is either phosphocholine or phosphoethanolamine.

The term “encapsulated” or “entrapped” as used herein means that the referred-to agent is located inside, or in the internal phase or core of, the liposome. It is possible for the agent to also be located in the external, outer or continuous phase.

The term “inclusion rate” as used here in refers to the percentage of a material or solution that has been encapsulated into liposomal vesicles relative to the starting material during a fabrication process.

“Pharmaceutical composition” refers to a composition of matter for pharmaceutical use. The terms “pharmaceutical composition” and “formulation” are used interchangeably.

“Polydispersity index” or “PdI” is a dimensionless number that is related to the size distribution of particles in a solution. PdI can be obtained by analysis of correlation data measured with the technique known as dynamic light scattering. This index is a number calculated from a simple two parameter fit to the correlation data (the cumulants analysis). The PdI is dimensionless and scaled such that values smaller than 0.05 are rarely seen other than with highly monodisperse standards. Values greater than 0.7 indicate that the sample has a very broad size distribution and is probably not suitable for size distribution measurement by dynamic light scattering (DLS) technique. The various size distribution algorithms work with data that falls between these two extremes. The calculations for these parameters are defined in the ISO standard document 13321:1996 E and ISO 22412:2008.

“Published material” means a medium providing information, including printed, audio, visual, or electronic medium, for example a flyer, an advertisement, a product insert, printed labeling, an internet web site, an internet web page, an internet pop-up window, a radio or television broadcast, a compact disk, a DVD, a podcast, an audio recording, or other recording or electronic medium.

“Safety” means the incidence or severity of adverse events associated with administration of a composition, including adverse effects associated with patient-related factors.

The term “effective amount” as used herein means an amount sufficient to achieve the desired result and accordingly will depend on the ingredient and its desired result. Nonetheless, once the desired effect is known, determining the effective amount is within the skill of a person skilled in the art.

The term “water” as used herein as an ingredient in the compositions of the application refers to pharmaceutically acceptable water.

The term “aqueous solution” as used herein means a solution wherein the solvent is primarily water, although small amounts, for example, less than 10, 9, 8, 7, 6, 5, 4, 3, 2 or 1% (v/v) of a non-aqueous solvent may be present.

The term “w/v” as used herein means the number of grams of solute in 100 mL of solution.

The term “w/w” as used herein means the number of grams of solute in 100 g of solution.

The term “turbid solution” refers to a solution that is cloudy or hazy in appearance due the presence of individual particles or suspended solids that, individually, are generally invisible to the naked eye.

The term “phase transition temperature” as used herein refers to the temperature required to induce a change in the lipid physical state from the ordered gel phase, where the hydrocarbon chains are fully extended and closely packed, to the disordered liquid crystalline phase, where the hydrocarbon chains are randomly oriented in the fluid.

In general, the “error bars” on the graphs represent the standard error of the mean value, whereas the top of the solid, shaded bar represents a single data value, which is the mean value of the distribution of data values.

The term “pharmaceutically acceptable” means compatible with the treatment of animals, in particular, humans.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilizing (i.e. not worsening) the state of disease, prevention of disease spread, delaying or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment. Treatment methods comprise administering to a subject a therapeutically effective amount of an active agent and optionally consists of a single administration, or alternatively comprises a series of applications. The length of the treatment period depends on a variety of factors, such as the severity of the condition, the age of the patient, the concentration of active ingredient or agent, the activity of the compositions described herein, and/or a combination thereof. The treatment period may also comprise cycles, for example, administration once daily for about two to seven days, followed by a period of rest for about 1 to 20 days, to constitute one cycle of treatment. Patients may be treated with more than one cycle, for example, at least two, three, four or five cycles. It will also be appreciated that the effective dosage of the agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient.

The term “subject” as used herein includes all members of the animal kingdom, including mammals, and suitably refers to humans.

A “user” means a subject such as a patient, a medical care worker, or a pharmaceutical supplier.

“Zeta potential” is a quantity which is related to the surface charge of a particle in a liquid and gives an indication of the potential stability of a solid dispersed in a liquid or a liquid dispersed in a liquid. If all the particles in suspension have a large negative or positive zeta potential then they will tend to repel each other and there is no tendency to flocculate. However, if the particles have low zeta potential values then there will tend to be less force to prevent the particles coming together and the particles may have a greater tendency to flocculate. Further information concerning the zeta potential may be found in Hunter, R. J. (1988) Zeta Potential In Colloid Science: Principles And Applications, Academic Press, UK.

When used with respect to methods of treatment and the use of compositions of the application, a subject “in need thereof” may be a subject who is suspected of having, has been diagnosed with or has been previously treated for the condition to be treated.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

### II. Liposomal Compositions

The present application is directed to liposomal compositions comprising chlorite, chlorate or a mixture thereof and methods for the preparation and use.

Accordingly, the present application includes a liposomal composition comprising liposomes having at least one lipid bilayer, and chlorite, chlorate or a mixture thereof encapsulated inside the liposomes, wherein the lipid bilayer is comprised of one or more suitable lipids. The liposomal composition comprises a plurality liposomes which in turn comprise bilayer-encapsulated, chlorite, chlorate or a mixture thereof. The liposomes comprise suitable lipids including, for example, phospholipids such as phosphatidylcholines having saturated and/or unsaturated fatty acid chains of a sufficient length and/or sphingolipids such as sphingomyelin, or appropriate mixtures of such lipids showing the desired behavior. These lipids could be of natural, semi-synthetic or synthetic source. The liposomes may further comprise additional components such as cholesterol or cholesterol sulfate to enhance the rigidity and reduce the permeability of the bilayer(s) and/or charged lipids to enhance the stability of the liposomes.

The present application also includes a liposome comprising at least one lipid bilayer and chlorite, chlorate or a mixture thereof entrapped inside the liposome, wherein the lipid bilayer is comprised of one or more suitable lipids.

The present application further includes a liposomal composition comprising encapsulated and non-encapsulated chlorite, chlorate or a mixture thereof, wherein the encapsulated chlorite, chlorate, or a mixture thereof is in the internal phase and the non-encapsulated chlorite, chlorate, or a mixture thereof is in the external phase of the liposome. In another embodiment the ratio of the chlorite, chlorate or a mixture thereof encapsulated within the at least one lipid bilayer and that present in the external phase is from about 100:1 to about 1:100, or from about 90:10 to 10:90. Alternately, the external phase may comprise a substance other than chlorite, chlorate or a mixture thereof, such as another therapeutic agent and/or sodium chloride.

In an embodiment of the application, the pH of the internal phase and external phase is the same. In another embodiment, the pH of the internal phase and external phase are different, for example, the pH in the inner phase may be pH of about 6 to about 13, about 8 to about 12.5, or about 10 to about 12, and the pH of the external phase may be approximately neutral such as about 6-8. In another embodiment, the liposomal composition has a pH difference between the inner core phase and outer continuous phase of about 1 to about 7, about 1 to about 5 or about 1 to about 3.

In another embodiment of the application, the internal phase and external phase of the liposomes are iso-osmotic or exhibit the same osmolarity. That is, the osmolarity of the internal phase and the external phase are within 1, 2, 3, 4, 5, 6 or 7% of each other. In an embodiment, by having the solutions on the inside and the outside of the liposome at the same or similar osmotic pressure, the amount of leaking of substances into or out of the liposome is reduced. In a further embodiment, the liposomal compositions are isotonic or iso-osmotic with respect to a subject's body fluids.

Alternately, in one embodiment the osmolarity of the internal phase and external phase of the liposomes are different. For example, the difference in osmolarity between the internal phase and the external phase may be about 8, 10, 15, 20, 25, 50, 100 or 200%.

In an embodiment the liposomal compositions of the present application show pharmaceutically-acceptable stability from about 3-48 months. In yet another embodiment, the pharmaceutically acceptable stability is achieved with storage of the composition at a temperature of about 5° C. to about 50° C. or about 5° C. to about 30° C.

In a further embodiment, the liposome composition may be lyophilized. Techniques for liposome lyophilization are well known, for example, Chen et al. (J. Control Release 2010 Mar. 19; 142(3):299-311) summarizes key factors determining the lyoprotective effect of freeze-dried liposomes.

In a further embodiment, the liposomal composition comprises liposomes that are unilamellar and/or multilamellar vesicles with an average diameter of about 80 nm to about 300 nm, about 90 nm to about 200 nm or about 100 nm to about 140 nm. In another embodiment, the liposomal composition comprises liposomes that are unilamellar and/or multilamellar vesicles with an average diameter of about 80 nm to about 15 microns, about 300 nm to about 12 microns or about 7 microns to about 10 microns. The nature of the particles size distribution can be unimodal or multimodal and the polydispersity index can be controlled by the method of manufacturing as would be known to a person skilled in the art, and determined based on the desired route of delivery.

Depending on the route of administration, it may be desirable to administer vesicles of a specific diameter. For instance, vesicles with a diameter of about 80 nm to about 300 nm may be useful for intravenous administration, while vesicles with a diameter of about 80 nm to about 10 microns may be useful for administration via inhalation, such as pulmonary, tracheal or nasal routes.

In a further embodiment, the liposome compositions may be sterilized. Non-limiting examples of suitable sterilization techniques include filtration, autoclaving, gamma radiation, and lyophilization (freeze-drying). In one embodiment, the sterilization is performed by filtration using a 220 nm filter.

Surprisingly, given that chlorite and chlorate contain only three or four, respectively, atoms, it has been discovered that it is possible to develop liposomes where the lipid bilayer forming the liposomal vesicle is substantially impermeable to chlorite and/or chlorate (i.e. the vesicles are ion-tight). This is despite the fact that other molecules, such as ethanol, glucose, ammonia and acetate are known to permeate through the lipid bilayers of liposomes. Thus, in one embodiment of the application the lipid bilayer is substantially impermeable to chlorite. In another embodiment of the application the lipid bilayer is substantially impermeable to chlorate. In a further embodiment, the lipid bilayer is substantially impermeable to chlorite and chlorate.

The ion-tight liposomes of the present application may be achieved with a single walled liposomal vesicle. In one embodiment of the application the ion-tight liposomes show stability over a commercially-acceptable shelf life (e.g. about 3-48 months at room or refrigeration temperature). In another embodiment the external (or outer phase) in which the ion-tight liposomal vesicles are dispersed may be engineered to contain a composition which is different from the encapsulated internal (or inner) phase. For example, the inner and outer phases may contain different concentrations of chlorite or, alternately, either phase may be substantially free of chlorite. As another example, the inner and outer phases may contain different concentrations of chlorate or, alternately, either phase may be substantially free of chlorate. In one embodiment of the application the pHs of the inner and outer phases of the ion-tight liposomal formulations are different. The outer phase may, for example, comprise a saline solution and may have a pH which is substantially different from the inner phase which may contain chlorite and/or chlorate. In one embodiment the outer phase may comprise sodium chloride, be substantially free of chlorite and/or chlorate and have an approximately neutral pH while the inner phase comprises chlorite and/or, and has a higher pH than the outer phase. Thus, the lipid bilayer forming the liposomal vesicle may additionally be substantially impermeable to H⁺ and OH⁻. Theoretically, it is possible to measure pH in the liposomes using a ¹³C-NMR based method. The ion-tight liposomal compositions of the instant application may be substantially free of chlorate or may comprise liposomal vesicles containing different concentrations of chlorate than the external phase. In another embodiment of the application the distribution of the diameters of the ion-tight vesicles comprising the liposomal composition has a range of values. In another embodiment the inner phase, external phase or both contain a plurality of substances. In yet other embodiments there is variation in the composition of material entrapped in different liposomes in the liposomal dispersion.

In an embodiment, the liposomal compositions of the present application do not contain hydrogen peroxide, isothiazolin and/or zinc ions. In another embodiment, the liposomal compositions do not contain liposomes prepared from lecithin. In a further embodiment, the liposomal compositions do not contain liposomes with chlorite and/or chlorite solely entrapped between the two lipids of a lipid bilayer. In still another embodiment, the liposomal compositions are substantially free of degradation products.

In a further embodiment, the liposomes of the present application are stable, that is the chlorite, chlorate or mixture thereof, does not leak from inside the liposomes for an acceptable shelf life, for example, as defined in a product specification approved by the Food and Drug Administration (FDA) or other government regulatory agencies. In one embodiment, the liposomes are stable at temperatures below about 30, 25, 20, 15, 10, 9, 8, 7 or 6° C., for a period of time sufficient to allow their use for an intended purpose. For example, for a period of time of 1 minute to 1 hour, 1 hour to 24 hours, 1 day to 30 days, 30 days to 200 days, 6 months to 1 year or further.

### A. Chlorite, Chlorate and Mixtures Thereof

The liposomal compositions of the present application comprise chlorite, chlorate or a mixture thereof. In one embodiment the liposomal compositions comprise chlorite. In one embodiment the liposomal compositions comprise stabilized chlorite. In one embodiment the liposomal compositions comprise chlorate. In one embodiment the liposomal compositions comprise chlorite and chlorate. In one embodiment the liposomal compositions comprise chlorite and are substantially free of chlorate. In one embodiment the liposomal compositions comprise chlorate and are substantially free of chlorite. In one embodiment the liposomal composition is a chlorate-free composition. In another embodiment the liposomal composition is a chlorite-free composition.

In another embodiment, the liposomal composition comprises a stabilized chlorite. Non-limiting examples of stabilized chlorite-based compositions include those described in U.S. Pat. Nos. 6,350,438, 6,251,372, 6,235,269, 6,132,702, 6,077,502 and 4,574,084, the contents of each of which is incorporated by reference in their entirety.

In another embodiment, the stabilized chlorite is a composition comprising chlorite, such as OXO-K993 (which comprises both chlorite and chlorate), or a composition comprising about 1-10%, 10-20%, 20-30%, 30-50% or 50-90% (w/v) OXO-K993. In a further embodiment, the stabilized chlorite is a composition comprising WF10 or is WF10.

In an embodiment, the stabilized chlorite is a composition comprising about 2% (w/v) OXO-K993. In a further embodiment, the stabilized chlorite is a composition comprising about 2% (w/v) OXO-K993, about 2% (w/v) glycerol and about 96% (w/v) water. Such compositions are sold commercially under the names of Oxovasin™ and Oxoferin™ (Nuvo Manufacturing, Wanzleben, Germany), where 1 ml of Oxovasin™ comprises about 0.85 mg (or about 0.085% w/v) of chlorite in 1.0 ml water. The pH of Oxovasin™ is between 10.75 and 11.90.

In an embodiment of the application, OXO-K993 is prepared using the following method:

Sodium chlorite (NaClO₂) and sodium hypochlorite (NaOCl) are mixed in a molar ratio of 4.8 to 1 in Water for Injection (WFI). The pH of the solution should be greater than pH 11.0. After addition of the catalyst, chlorylsulfuric acid [ClO₂⁺][HSO₄], to this mixture the following reaction can be observed:

2ClO₂⁻+OCl⁻+2H⁺→2ClO₂+Cl⁻+H₂O (1)

The pH of the solution decreases. A portion of the chlorite is oxidized to chlorine dioxide (ClO₂) in the redox process described by Equation (1). In an equilibrium reaction, the developing chlorine dioxide forms an intense brown charge-transfer complex with the excess unoxidized chlorite, as shown in Equation (2):

ClO₂+ClO₂⁻ [symbol] [Cl₂O₄]⁻ (2)

9.65 mmol (per kg of the reaction solution) of sodium carbonate peroxohydrate (2 Na₂CO₃.3H₂O₂) is then added to the solution. Upon addition of sodium carbonate peroxohydrate, part of the chlorine dioxide is reduced back to chlorite, and oxygen is formed simultaneously:

2ClO₂+H₂O₂+2OH⁻→2ClO₂⁻+O₂+2H₂O (3)

After a suitable time, for example 15 minutes, 102 mmol (per kg of the reaction solution) of sodium peroxide (Na₂O₂) is added to the solution, which becomes completely decolorized as the remaining chlorine dioxide is reduced completely to chlorite. From sodium peroxide, oxygen evolves in a slow process that typically requires at least 4 weeks (Equation 4). Simultaneously, hydroxyl ions are formed, resulting in a high pH value (pH>13) of the solution, which thereby stabilizes the active substance chlorite.

2O₂²⁻+2H₂O→O₂+4OH⁻ (4)

The final reaction product, OXO-K993, resulting from this synthesis is a stable aqueous solution, which contains the anions chlorite (4.25%), chloride (2.0%), chlorate (1.5%), and sulfate (0.7%), and sodium as the cation as well as a sodium carbonate/sodium hydroxide buffer system which maintains the alkaline pH of the formulation.

The skilled artisan will recognize that any chemically-stabilized chlorite solution, including derivatives of OXO-K993, WF10, Oxovasin™ or other chlorite-based or chlorate-based solutions and their derivatives, are well within the scope of the application. These solutions can be used in the compositions, methods and uses of the present application and, as such, the scope of the application is not necessarily limited to use of the products described herein.

OXO-K993 and its derivatives (WF10, Oxoferin, Oxovasin) are also examples of compositions comprising a mixture of ions, since the formulations comprise a combination of chlorite, chloride, chlorate, sulfate and sodium ions. In one embodiment of the application, a composition comprising a mixture of ions is a composition comprising at least two different types of anions (chlorate and chlorite). In another embodiment of the application, the composition may comprise at least three different types of anions. In a further embodiment of the application, the composition may comprise at least four, at least 5 or at least 6 different types of anions.

In a further embodiment, the liposomal composition comprises encapsulated chlorite present in an amount of about 0.01% (w/w) to about 50% (w/w), about 0.1% (w/w) to about 20% (w/w), or about 0.5% (w/w) to about 10% (w/w) of the total encapsulated ion content of the composition.

In yet a further embodiment, the liposomal composition comprises encapsulated chlorate present in an amount of about 0.01% (w/w) to about 50% (w/w), about 0.1% (w/w) to about 20% (w/w) or about 0.5% (w/w) to about 10% (w/w) of the total encapsulated ion content of the composition.

In still a further embodiment, the liposomal composition comprises an encapsulated mixture of chlorate and chlorite ions present in an amount of about 0.01% (w/w) to about 50% (w/w), about 0.1% (w/w) to about 20% (w/w) or about 0.5% (w/w) to about 10% (w/w of the total encapsulated ion content of the composition. Any composition of matter containing chlorite and/or chlorate ions also has at least one counter ion to maintain charge neutrality. Thus, according to one embodiment of the present application, the liposomal compositions comprise one or more cations. Non-limiting examples of possible cations include alkali metal cations (such as sodium or Na⁺) and alkaline earth cations. In another embodiment, the liposomal compositions comprising chlorite ions, chlorate ions or a mixture thereof, further comprise sodium and/or potassium counter ions.

In a further embodiment, the liposomal composition comprises encapsulated chlorite present in an amount of about 0.1% (w/w) to about 100% (w/w), about 1% (w/w) to about 90% (w/w), about 2% (w/w) to about 80% (w/w), about 3% (w/w) to about 70% (w/w), about 4% (w/w) to about 60% (w/w) or about 5% (w/w) to about 50% (w/w) of the total encapsulated anion content of the composition.

In yet a further embodiment, the liposomal composition comprises encapsulated chlorate present in an amount of about 0.1% (w/w) to about 100% (w/w), about 1% (w/w) to about 90% (w/w), about 2% (w/w) to about 80% (w/w), about 3% (w/w) to about 70% (w/w), about 4% (w/w) to about 60% (w/w) or about 5% (w/w) to about 50% (w/w) of the total encapsulated anion content of the composition.

In still a further embodiment, the liposomal composition comprises an encapsulated mixture of chlorate and chlorite ions present in an amount of about 0.1% (w/w) to about 100% (w/w), about 1% (w/w) to about 90% (w/w), about 2% (w/w) to about 80% (w/w), about 3% (w/w) to about 70% (w/w), about 4% (w/w) to about 60% (w/w) or about 5% (w/w) to about 50% (w/w) of the total encapsulated anion content of the composition.

The skilled artisan will recognize that the ratio of anions in the liposomal composition may be adjusted qualitatively or quantitatively based on its intended use. For example, the ratio of anions in the composition may be adjusted for the treatment of a specific disease, disorder or condition. In one embodiment the ratio of the first type of anion to the second type of anion is from about 100:1 to about 1:100, from about 90:10 to 10:90, from about 1:1 to about 3:1 (w/w) or about 2:1 (w/w). In an embodiment, the first type of anion is chlorite and the second type of anion is chlorate.

In an embodiment, the pH of the chlorite, chlorate or a mixture thereof is greater than about 8, greater than about 9, or greater than about 10. In an embodiment the pH is about 8 to about 14, about 8 to about 13, about 9 to about 12.5, or about 10 to about 12.

In another embodiment, the pH of the chlorite, chlorate or a mixture thereof is less than about 8, less than about 7, or less than about 6. In an embodiment the pH is about 5 to about 8, about 6 to about 7, or about 7 to about 7.5.

In a further embodiment, the liposomal composition may contain two pH values, the first value relates to an inner, internal or encapsulated phase pH and the second value relates to an external or outer phase pH. For example, the pH of the inner phase of a liposomal composition may be about 6-13, while the pH of the outer phase of a liposomal composition may be about 6-8. Accordingly, in one embodiment, there is a pH difference of the inner phase and outer phase of between about 1-7, about 1-6, about 1-5, about 1-4, about 1-3, about 1-2 or about 1.

The chlorite and chlorate for use in the present application may be obtained from any available source and are commercially available. In an embodiment, the chlorite or chlorate is a sodium salt, although a person skilled in the art would appreciate that other metal salts can be used.

The amount of chlorite, chlorate or mixture thereof in the liposomal compositions of the present application is typically the maximum amount that can be entrapped in the liposome using the preparation method. In an embodiment, the chlorite, chlorate or mixture thereof is entrapped with an efficiency or inclusion rate of about 1% to about 50%, about 2% to about 25% or about 5% to about 15%.

### B. Lipids

The lipids comprised in the liposomes of the present application are selected from those that are suitable for the entrapment of ions having a pH of about 5 to about 14, about 6 to about 13, about 8 to about 12.5, or about 10 to about 12. Factors determining the suitability of a lipid for preparing the liposomes of the present application include: (1) ability to form lipid bilayers in an aqueous medium; (2) ability to encapsulate appreciable amounts of ions; (3) impermeability of formed liposomes to leakage of chlorite and/or chlorate ions; (4) resistance of formed liposomes to hydrolysis in alkaline environments (e.g. if inner pH is 8 or above); and/or (5) ability of formed liposomes to remain stable for an acceptable period of time at storage temperatures falling within the range of about 5° C. to about 50° C.

The examples provided herein demonstrate that not all lipids are suitable for preparing stable liposomes for the entrapment of ions, such as chlorate or chlorite. Surprisingly, through the teachings of the present application, in one embodiment it has been found that suitable lipids are selected from phospholipids and sphingolipids. In another embodiment, suitable phospholipids are selected from a phosphatidylcholine (PC) comprising saturated or unsaturated fatty acyl chains of a sufficient length, such as greater than 12, greater than 13 or greater than 14 carbon atoms, and the suitable sphingolipids are selected from sphingomyelin (SM). In another embodiment, the suitable lipids are selected from sphingomyelin (SM), 1,2-dipalmitoyl-2-sn-glycero-3-phosphocholine (DPPC), 1,2-dimyristoyl-2-sn-glycero-3-phosphocholine (DMPC), hydrogenated soybean or egg yolk phospholipids and mixtures thereof. In a further embodiment, the suitable lipids are selected from those that form liposomes that are impermeable to leakage of ions at about 5° C. or above, at about 23° C. or above, at about 37° C. or above, or at about 50° C. or above. In another embodiment, the liposomes of the application may or may not be impermeable at or near the Phase Transition Temperature (PPT) of the lipids from which they are formed. The PPTs for various lipids are known, for example, 1-palmitoyl-2-oleoyl-sn-3-glycero-3-phosphocholine (−2° C.), 1,2-dimyristoyl-2-sn-glycero-3-phosphocholine (23° C.), sphingomyelin (37° C.), 1,2-dipalmitoyl-2-sn-glycero-3-phosphocholine (41° C.), and hydrogenated soybean or egg yolk phospholipids (e.g. 50° C. for hydrogenated soybean phospholipid).

The liposomes of the application may include two or more types of suitable lipids. When the liposomes comprise two types of suitable lipids the lipids may be present in a molar ratio of 20:1 to 1:1, 15:1 to 5:1 or 10:1 to 9:1. In one embodiment, the two types of suitable lipids are selected from sphingomyelin (SM), 1,2-dipalmitoyl-2-sn-glycero-3-phosphocholine (DPPC), 1,2-dimyristoyl-2-sn-glycero-3-phosphocholine (DMPC), and hydrogenated soybean or egg yolk phospholipids.

The compositions of the application may further comprise at least one additional component such as cholesterol or cholesterol sulfate to enhance the rigidity and/or reduce the permeability of the lipid bilayer(s). The at least one additional component can be present in an amount from about 0.1 to about 50%, about 1 to about 30%, about 5 to about 25% or about 10 to about 20% of the total lipid content.

It is another embodiment that the composition of the application further comprising at least one additional component that provides the liposomes with a zeta potential that reduces aggregation of the liposomes, such as a zeta potential that is at least more positive than about +0.5 mV or more negative than about −0.5 mV. In an embodiment, the zeta potential is from about +1 mV to +50 mV, about +10 mV to +40 mV or about +15 mV to +30 mV. In another embodiment, the zeta potential is from about −1 mV to −50 mV, about −10 mV to −40 mV or about −15 mV to −30 mV. In a further embodiment, the at least one additional component that provides the liposomes with a zeta potential that reduces aggregation of the liposomes is a charged lipid. In another embodiment, the lipids used for preparing the liposomes of the present application include at least one charged lipid. While not wishing to be limited by theory, the presence of a charged lipid causes adjacent liposomes to repel as they approach each other, reducing the amount of contact between liposomes and therefore the amount of fusion or aggregation of liposomes that causes an increase in their size. Accordingly, it is an embodiment of the application that the liposomes comprise charged lipids that provide a zeta potential that allows them to repel each other, for example at least more positive than +1 mV, +5 mV, +10 mV, +15 mV, +25 mV, +35 mV or +45 mV or more negative than −1 mV, −5 mV, −10 mV, −15 mV, −25 mV, −35 mV or −45 mV.

In an embodiment, the charged lipid is a negatively charged lipid, such as a phosphatidyl glycerol, a phosphatidyl ethanolamine, a phosphatidyl serine, or a phosphatidic acid. However, the charged lipid need not be a phospholipid or sphingolipid.

Negatively charged lipids are preferable for preparing the liposome of the present application, however, the liposomes of the present application do not exclude the use of positively charged lipids. Accordingly, in one embodiment the liposomes of the application may include at least one positively charged lipid. For example, 1,2-dilauroyl-sn-glycero-3-ethylphosphocholine chloride salt EPC (chloride salt), N-[1-(2,3-dioleyloyx) propyl]-N—N—N-trimethyl ammonia chloride (DOTMA), dimethyldioctadecyl ammonium bromide salt (DDAB) and other pH-sensitive cationic phospholipids. However, the charged lipid need not be a phospholipid or sphingolipid. For example, other charged lipids such as N-[1-(2,3-dioleoyloxy)propyl]-N,N,N-trimethylammonium methyl-sulfate (DOTAP) may be used to prepare the liposomes of the application.

In one embodiment, the charged lipid is present in an amount that provides a molar ratio of uncharged:charged lipids of 20:1 to 1:1, 15:1 to 5:1 or 10:1 to 9:1. In a further embodiment, the presence of a charged lipid in the liposomes improves the stability of the resulting liposome, in particular in comparison to an otherwise identical liposome lacking the charged lipid.

In an embodiment, the suitable lipids that are comprised in the liposomes of the present application are selected from those listed in Table 1.

In an embodiment, the suitable lipids are selected from 1,2-dipalmitoyl-2-sn-glycero-3-phosphocholine (DPPC), 1,2-dimyristoyl-2-sn-glycero-3-phosphocholine (DMPC), 1-myristoyl-2-stearoyl-sn-glycero-3-phosphocholine (MSPC), 1-palmitoyl-2-myristoyl-sn-glycero-3-phosphocholine (PMPC), 1,2-distearoyl-sn-glycero-3-phosphocholine (DSPC), hydrogenated Egg PC (HEPC), 1-palmitoyl-2-stearoyl-sn-glycero-3-phosphocholine (PSPC), 1-stearoyl-2-myristoyl-sn-glycero-3-phosphocholine (SMPC), 1-stearoyl-2-oleoyl-sn-glycero-3-phosphocholine (SOPC), 1-stearoyl-2-palmitoyl-sn-glycero-3-phosphocholine (SPPC), diarachidoylphosphatidylcholine (DAPC), 1,2-dibehenoyl-sn-glycero-3-phosphocholine (DBPC), 1,2-dierucoyl-sn-glycero-3-phosphocholine (DEPC), 1,2-Dioleoyl-sn-glycero-3-phosphocholine (DOPC), hydrogenated soybean or egg yolk phospholipids, and mixtures thereof. In another embodiment, the lipid is sphingomyelin (SM) or milk sphingomyelin. In a further embodiment, the lipid is a hydrogenated soybean or egg yolk phospholipid. In another embodiment, the suitable lipids are selected from 1,2-dipalmitoyl-2-sn-glycero-3-phosphocholine (DPPC), 1,2-dimyristoyl-2-sn-glycero-3-phosphocholine (DMPC), hydrogenated soybean or egg yolk phospholipids, and mixtures thereof, in combination with a charged lipid or another lipid. In a further embodiment, the charged lipid is a negatively charged phospholipid. In yet another embodiment, the negatively charged phospholipid is a phosphatidyl glycerol, such as the salts of 1,2,-dipalmitoyl-sn-glycero-3-phosphoglycerol (DPPG), 1,2-dimyristoyl-sn-glycero-3-phosphoglycerol (DMPG), 1,2-dioleoyl-sn-glycero-3-phosphoglycerol (DOPG), or 1,2-distearoyl-sn-glycero-3-phosphoglycerol (DSPG), a phosphatidyl ethanolamine such as the salts of 1,2-distearoyl-sn-glycero-3-phosphoethanolamine (DSPE), or 1,2-distearoyl-sn-glycero-3-phosphoethanolamine-methyl-polyethyleneglycol (MPEG-DSPE), or a mixture thereof. In a further embodiment, the phosphatidyl glycerol is DPPG or DSPG or a mixture thereof.

It is an embodiment of the application that when a mixture of lipids is used in the liposomes, the lipid that is present in the majority amount is a lipid with a PTT that is greater than about 5° C. to about 30° C. Such lipids are those, for example, comprising a carbon chain that contains more than 12 contiguous carbon atoms.

In one embodiment of the application, particular ratios of lipids are desirable. In another embodiment, the lipids are selected from 1,2-dipalmitoyl-2-sn-glycero-3-phosphocholine (DPPC) and 1,2-dimyristoyl-2-sn-glycero-3-phosphocholine (DMPC) in a molar ratio of 9:1. In a further embodiment, the lipids are selected from 1,2-dipalmitoyl-2-sn-glycero-3-phosphocholine (DPPC) and 1,2-dimyristoyl-sn-glycero-3-phosphoglycerol (DMPG) in a molar ratio of 9:1. In yet another embodiment, the lipids are selected from 1,2-dipalmitoyl-2-sn-glycero-3-phosphocholine (DPPC) and 1,2,-dipalmitoyl-sn-glycero-3-phosphoglycerol (DPPG) in a molar ratio of 9:1. In still another embodiment, the suitable lipid is pure DPPC.

Lipids with a particular PPT may also be selected for preparation of the liposomal composition of the application. Accordingly, it is an embodiment of the application that the liposomes comprise lipids with a PPT that is above the body temperature of an animal. It is another embodiment of the application that the liposomes comprise lipids with a PPT that is below the body temperature of an animal.

Such materials are commercially available, for example from Avanti Polar Lipids, (Alabaster, Ala. USA), LIPOID GmbH (Germany) or may be prepared using methods known in the art.

### C. Liposome Surface Modifying Agents

To increase the circulation time of the liposomes of the present application, it is an embodiment that the surface of the liposomes, or the lipid bilayer, is modified with molecules that increase hydrophilicity, such as with hydrophilic polymers.

In an embodiment, the hydrophilic polymer is poly-(ethylene glycol) (PEG). The presence of PEG on the surface of the liposomes has been shown to extend blood-circulation time while reducing mononuclear phagocyte system (MPS) uptake.

Surface modification of liposomes with PEG can be achieved in several ways: by physically adsorbing the polymer onto the surface of the vesicles, by incorporating the PEG-lipid conjugate during liposome preparation, or by covalently attaching reactive groups onto the surface of preformed liposomes.

Grafting PEG onto liposomes has demonstrated several biological and technological advantages. The most significant properties of PEGylated vesicles are their strongly reduced MPS uptake and their prolonged blood circulation and thus improved distribution in perfused tissues. Moreover, the PEG chains on the liposome surface avoid vesicle aggregation, improving their stability. PEG-modified liposomes are also often referred to as “shielded” liposomes. Doxil™ (doxorubicin HCl liposome injection) is a liposome-enclosed doxorubicin, with adjunct polyethylene glycol (PEG) utilized to avoid the reticuloendothelial system (RES) and prolong drug circulation time (see Vail D M, Amantea M A, Colbern G T, et al., “Pegylated Liposomal Doxorubicin. Proof of Principle Using Preclinical Animal Models and Pharmacokinetic Studies.” Semin Oncol. (2004) 31 (Suppl 13): 16-35).

In liposomes composed of phospholipids and cholesterol, the ability of PEG to increase the circulation lifetime of the vehicles has been found to depend on both the amount of grafted PEG and the length or molecular weight of the polymer Allen T M, et al. Biochim Biophys Acta. 1989, 981:27-35). In most cases, the longer-chain PEGs have produced the greatest improvements in blood residence time. In an embodiment, the PEG has a molecular weight of about 1500 to about 5000.

Other hydrophilic polymers that may be used to surface-modify the liposomes of the present application include polymers that are water soluble, hydrophilic, have a flexible main chain, and biocompatibility. In an embodiment, the hydrophilic polymers are selected from PEG, poly(vinyl pyrrolidone) (PVP), poly(acryl amide) (PAA), phosphatidyl polyglycerols, poly[N-(2-hydroxypropyl) methacrylamide], L-amino-acid-based biodegradable polymers, polyvinyl alcohol (e.g. PVA with a MW of about 20,000), poly(2-methyl-2-oxazoline), poly(2-ethyl-2-oxazoline) and mixtures thereof.

In another embodiment, the surface of the liposome is modified with a ganglioside and/or a sialic acid derivative, such as monosialoganglioside (GM1). Several glycolipids have been tested in studies of MPS uptake of liposomes after iv injection: the glycolipid GM1 (a brain-tissue-derived monosialoganglioside) significantly decreased MPS uptake when incorporated on the liposome surface, and the formulation remained in blood circulation for several hours. GM1 grafted liposomes with a diameter in the 90-200 nm range have longer blood retention, with consequent accumulation in tumor tissues, than those out of this size range. GM-coated liposomes may be useful for oral administration and delivery to the brain. In particular, it has been suggest that among liposomal formulations used as oral drug carriers, those containing GM1 and GM type III have better possibilities of surviving through the gastrointestinal tract (Taira M C, et al. Drug Deliv. 2004; 11:123-8). Further, there was a higher brain-tracer uptake for GM1 liposomes than for control liposomes in the cortex, basal ganglia, and mesencephalon of both hemispheres; conversely, no significant changes were observed in liver uptake or blood concentration of the tracer (Mora M, et al. Pharm Res. 2002; 19:1430-8).

In addition to PEG-modified liposomes, researchers developed a variety of other derivatized lipids. These derivatized lipids could also be incorporated into liposomes. See, for example: International Patent Application WO 93/01828; Park Y S, Maruyama K, Huang L. “Some negatively charged phospholipids derivatives prolong the liposome circulation in vivo.” Biochimica et Biophysica Acta (1992) 1108: 257-260; Ahl et al., Biochimica Biophys. Acta (1997) 1329: 370-382.

### D. Targeting Moieties

To increase liposomal drug accumulation in desired tissues, producing higher and more selective therapeutic activity, it is an embodiment that the liposomes of the present application are modified by attaching cell-specific targeting moieties to their surface to facilitate their association with a specific cell or tissue type. Targeting moieties include, for example, monoclonal antibodies (MAb) or fragments, peptides, growth factors, glycoproteins, carbohydrates, or receptor ligands, or mixtures thereof. Exemplary targeting moieties include, but are not limited to, transferrin, folic acid, folate, hyaluronic acid, sugar chains (e.g., galactose, mannose, etc.), fragments of monoclonal antibodies, asialoglycoprotein, etc., as well as other targeting factors known to the skilled artisan, and mixtures thereof. In particular embodiments, the targeting factor is a protein, peptide or other molecule, directed to a cell surface receptor (e.g., transferrin, folate, folic acid, asialoglycoprotein, etc.).

Examples of lipid compositions that include targeting factors include those disclosed in U.S. Pat. Nos. 5,049,390; 5,780,052; 5,786,214; 6,316,024; 6,056,973; 6,245,427; 6,524,613; 6,749,863; 6,177,059; and 6,530,944; U.S. Pat. App. Publication. Nos. 2004/0022842; 2003/0224037; 2003/143742; 2003/0228285; 2002/0198164; 2003/0220284; 2003/0165934; and 2003/0027779; International Patent Application Nos. WO 95/33841; WO 95/19434; WO 2001037807; WO 96/33698; WO 2001/49266; WO 9940789; WO 9925320; WO 9104014; WO 92/07959; EP 1369132; and JP 2001002592; and in linuma H, et al. Int J Cancer, 2002, 99:130-137; Ishida 0, et al. Pharmaceutical Research, 2001, 18:1042-1048; Holmberg et al. Biochem. Biophys. Res. Comm. 1989, 165(3):1272-1278; Nam et al., J. Biochem. Mol. Biol. 1998, 31(1): 95-100; and Nag et al. J. Drug Target. 1999, 6(6): 427-438.

In particular, linuma et al. (ibid) developed a Tf-PEG-liposome, with transferrin (Tf) attached at the surface of the liposome and showed that a greater number of liposomes were bound to the surface of the tumor cells, and there was a greater uptake of liposomes by the tumor cells for Tf-PEG-liposome as compared to PEG-liposome (Inuma et al., ibid; Ishida et al., ibid).

Examples of specific targeting moieties and their therapeutic targets are as follows: Anti-HER2 (trastuzumab)—breast/ovarian cancer; Anti-EGF—solid tumors; Anti-CD19—lymphoma; Anti-CD22—B-cell lymphoma; Anti-beta1 integrin—cancer cells; Anti-GD2—neuroblastoma; Anti-GAH—gastric, colon and breast cancer; Folic Acid—cancer cells; Transferrin—cancer cells; Anisamide—breast, melanoma and prostate cancer; Vasoactive intestinal peptide 28-mer—imaging of breast cancer cells; RGD—neuroblastoma, melanoma and colon cancer; Angiogenic homing peptide—melanoma, sarcoma and colon cancer.

In an embodiment, the liposomal compositions of the application are injected into a subject and heat is applied to the relevant body part to enable targeted delivery of the vesicle contents. This is particularly useful for liposomes that are stable at body temperature. Known methods such as the hyperthermic treatment of cancer may benefit from targeted delivery of the compositions of the application.

In an embodiment, the liposomes of the present application comprise a surface-modifying hydrophilic peptide and a targeting moiety. In another embodiment, these liposomes are prepared by mixing a PEG derivative of a suitable lipid containing a maleimide group at the end of the PEG chain into the liposome formulation. After liposome preparation, targeting moieties comprising a nucleophilic N, O, and/or S atom, for example, are joined via surface linkage to the maleimide group of the aforementioned PEG-liposome, obtaining a stable bond. Alternatively, commercially pre-loaded long-circulating liposomes are modified by post-insertion of the targeting moiety.

### E. Other Components

In further embodiments of the present application, the liposomal compositions and liposomes of the application further comprise other additives or agents that are desired for particular applications. Such additives or agents include, but are not limited to, humectants, solvents, antibiotics, dyes, perfumes, fragrances and the like. In one embodiment, the compositions comprise an anti-oxidant. In an embodiment, the anti-oxidants for use in the present application include butylated hydroxytoluene, butylated hydroxyanisole, ascorbyl linoleate, ascorbyl dipalmitate, ascorbyl tocopherol maleate, calcium ascorbate, carotenoids, kojic acid and its pharmaceutically acceptable salts, thioglycolic acid and its pharmaceutically acceptable salts (e.g., ammonium), tocopherol, tocopherol acetate, tocophereth-5, tocophereth-12, tocophereth-18, or tocophereth-80, or mixtures thereof.

### III. Methods of Preparation

The liposomes of the present application may be prepared using any known method for the preparation of liposomes, for example, thin-film methods, sonication, extrusion, high pressure/homogenization, microfluidization, detergent dialysis, ethanol injection method, ethanol injection method comprising the crossflow technique, calcium-induced fusion of small liposomes vesicles and ether-infusion methods. Such methods are well-known in the art (see, for example, “Liposome Technology”, G. Gredoriadis (Ed.), 1991, CRC Press: Boca Raton, Fla.; D. Deamer and A. D. Bangham, Biochim. Biophys. Acta, 1976, 443: 629-634; Fraley et al., Proc. Natl. Acad. Sci. USA, 1979, 76: 3348-3352; F. Szoka et al., Ann. Rev. Biophys. Bioeng., 1980, 9: 467-508; Hope et al., Biochim. Biophys. Acta, 1985, 812: 55-65; L. D. Mayer et al., Biochim. Biophys. Acta, 1986, 858: 161-168; Hope et al., Chem. Phys. Lip., 1986, 40: 89-1-7; K. J. Williams et al., Proc. Natl. Acad. Sci., 1988, 85: 242-246; Wagner and Vorauer-Uhl, Journal of Drug Delivery, 2011, pp. 1-9; Wagner et al., Journal of Liposome Research, 2002, 12(3): 259-270; Wagner et al., European Journal of Pharmaceutics and Biopharmaceutics, 2002, 54:213-219 and U.S. Pat. Nos., 4,217,344; 4,235,871; 4,241,046; 4,356,167; 4,485,054; 4,551,288; 4,663,161; 4,737,323; 4,752,425; 4,774,085; 4,781,871; 4,877,561; 4,927,637; 4,946,787; 5,190,822; 5,206,027; 5,498,420; 5,556,580 and 5,700,482).

In one embodiment, the liposomes are prepared using the traditional thin-film method. In this method, the bilayer-forming elements are mixed with a volatile organic solvent or solvent mixture (e.g., chloroform, ether, methanol, ethanol, butanol, cyclohexane, and the like). The solvent is then evaporated (e.g., using a rotary evaporator, a stream of dry nitrogen or argon, or other means) resulting in the formation of a dry lipid film. The film is then hydrated with an aqueous medium containing chlorite, chlorate or a mixture thereof. The hydration steps used influence the type of liposomes formed (e.g., the number of bilayers, vesicle size, and entrapment volume). The hydrated lipid thin film detaches during agitation and self-closes to form large, multilamellar vesicles (MLV) of heterogeneous sizes. The size distribution of the resulting multilamellar vesicles can be shifted toward smaller sizes by hydrating the lipids under more vigorous agitation conditions or by adding solubilizing detergents, such as deoxycholate. Alternatively or additionally, the vesicle size can be reduced by sonication, freeze/thawing or extrusion (see below).

Large unilamellar vesicles (LUVs) can be prepared from the MLV using any of a variety of methods. For example, extrusion of MLVs through filters can provide LUVs whose sizes depend on the filter pore size used. In such methods (described, for example, in U.S. Pat. No. 5,008,050), the MLV liposome suspension is repeatedly passed through the extrusion device resulting in a population of LUVs of homogeneous size distribution. When lipids having a gel to liquid crystal transition above ambient temperature are employed, an extruder having a heated barrel (or thermojacket) may be employed. LUVs may be exposed to at least one freeze-and-thaw cycle prior to the extrusion procedure as described by Mayer et al. (Biochim. Biophys. Acta, 1985, 817: 193-196).

Other methods for the preparation of unilamellar vesicles rely on the application of a shearing force to an aqueous dispersion of liposomes. Such methods include sonication and homogenization. Sonicating a liposome suspension using either a bath or probe sonicator leads to a progressive size reduction down to small unilamellar vesicles less than 50 nm in size. The size of the liposomal vesicles can be determined by quasi-elastic light scattering (QELs) (V. A. Bloomfield, Ann. Rev. Biophys. Bioeng., 1981, 10: 421-450). In a typical homogenization procedure, multilamellar vesicles are repeatedly circulated through a standard emulsion homogenizer at a pressure of 3,000 to 14,000 psi, preferably 10,000 to 14,000 psi and at a temperature corresponding to the gel-liquid crystal transition temperature of the lipid with the highest Tc, until selected liposome sizes, typically between about 100 and 500 nm, are observed.

Other techniques for preparing LUVs include reverse phase evaporation (U.S. Pat. No. 4,235,871) and infusion procedures, and detergent dilution. For example, unilamellar vesicles can be produced by dissolving lipids in chloroform or ethanol and then injecting the lipids into a buffer, causing the lipids to spontaneously aggregate and form unilamellar vesicles. Alternatively, phospholipids can be solubilized into a detergent (e.g., cholates, Triton-X, or n-alkylglucosides). After formation of the solubilized lipid-detergent micelles, the detergent is removed by dialysis, gel filtration, affinity chromatography, centrifugation, ultrafiltration or any other suitable method.

In one embodiment, the present application includes a method of preparing liposomes comprising chlorite, chlorate or a mixture thereof entrapped within at least one lipid bilayer, wherein the lipid bilayer is comprised of one or more suitable lipids, the method comprising:

(a) adding an aqueous solution of chlorite, chlorate or a mixture thereof to a vessel having a film of the one or more lipids on at least a portion of an inner surface;
(b) agitating the vessel under conditions sufficient to wholly or partially remove the film from the inner surface to provide a turbid solution comprising the chlorite- and/or chlorate-entrapped liposomes;
(c) treating the turbid solution to reduce the average diameter of the liposomes to a desired amount, for example, between about 50 nm and about 300 nm; and
(d) optionally treating the liposomes to remove chlorite, chlorate or a mixture thereof from a solution external to the liposomes.

In another embodiment, the liposomes of the present application are prepared using an ethanol injection method, for example, as described in Wagner and Vorauer-Uhl, Journal of Drug Delivery, 2011, pp. 1-9, the relevant portions of which are incorporated herein by reference. I n a further embodiment, the liposomes are prepared using an ethanol injection method by crossflow technique, for example, as described in Wagner et al., Journal of Liposome Research, 2002, 12(3): 259-270, the relevant portions of which are incorporated herein by reference.

In an embodiment, the molar ratio of the one or more suitable lipids to the chlorite, chlorate or a mixture thereof in (a) is about 0.01:1 to about 10000:1, about 0.1:1 to about 5000:1, about 0.5:1 to about 2500:1, about 1:1 to about 1000:1, or about 0.1:1 to about 100:1.

The lipids used for the preparation of the liposomes of the present application can be purchased as solutions, in particular from Avanti Lipids. LIPOID AG delivers the lipids undiluted in solid state. In certain embodiments, the concentration of the solution is about 10 mg to about 100 mg of lipid per milliliter of solution. The amount of this solution that is used for the preparation of the liposomes will vary depending on the amount of lipids needed, which will depend on the desired sample volume. Should pure, undissolved lipids be used, they are dissolved in an appropriate volume of chloroform or another suitable organic solvent.

To obtain a vessel having a film of the one or more lipids on at least a portion of an inner surface, it is an embodiment that the vessel is treated, with agitation, under reduced pressure and at a temperature near or above the phase transition temperature (PPT) of all of the one or more lipids, to remove the solvent. Note, it is not necessary to stay above PTT in the evaporation step. For chloroform solutions, a water bath of 37° C. can be applied, which is below the PTT of DPPC (41° C.) and that of the hydrogenated soybean phospholipid (50° C.). In an embodiment, the solvent is removed using a rotary evaporator with the vessel being maintained at a temperature above the lipid phase transition temperature of all of the one or more lipids.

The aqueous solution is added to the vessel and the vessel is agitated under conditions sufficient to remove the film from the inner surface of the vessel to provide a turbid solution/dispersion comprising liposomes. In an embodiment, the conditions sufficient to remove the film comprise shaking the vessel and heating the vessel to a temperature above the lipid phase transition temperature of all of the one or more lipids. In another embodiment the conditions further comprise shaking at a temperature above the lipid phase transition temperature of all of the one or more lipids for about 1 minute to about 2 hours, or about 5 minutes to about 1 hour. In this step, rehydration of the one or more lipids occurs with formation of liposomes and entrapment of the chlorite, chlorate or a mixture thereof solution. These, so called primary liposomes are large multilamellar vesicles (LMV) with the solution both inside (inner phase) and outside (outer phase) the liposomes. At this stage the sample is a turbid, milky-white solution/dispersion.

Treating the turbid solution to reduce the average diameter of the liposomes to between about 50 nm and about 300 nm can be done using any known means to reduce the size of liposomes. In an embodiment of the application a combination of freeze-thaw cycles and extrusion methods are used. In another embodiment, only extrusion methods are used.

For the freeze-thaw cycle, it is an embodiment that the sample is transferred to a suitable vessel (if needed), such as a cryotube, and the vessel, immersed in liquid nitrogen and subsequently thawed in a water bath above the lipid phase transition temperature of all of the one or more lipids. In an embodiment, at least 2, 3, 4, 5, 6, 7, 8, 9, or 10 cycles are carried out. While not wishing to be limited by theory, the freezing results in destruction of the primary liposomes and thawing, above the lipid phase transition temperature of all of the one or more lipids, results in spontaneous re-formation of liposomes with a smaller average diameter. However, freeze-thaw methods are optional.

In an embodiment of the application extrusion methods are carried out by squeezing the liposome sample through at least one about 50 nm to about 200 nm, filter disk. In an embodiment, the squeezing is done by applying a pressure to an extruder comprising the appropriate filter. In a further embodiment, extrusion is carried out at a temperature above the lipid phase transition temperature of all of the one or more lipids and is repeated at least 2, 3, 4, 5, 6, 7, 8, 9, or 10 times.

In an embodiment, the use of extrusion methods only provides unilamellar liposomes with an average diameter of about 50 nm to about 150 nm or about 100 nm. In another embodiment, a sonication method can be used to reduce the particle size or convert MLVs into LUV/SUVs.

In another embodiment, the methods of the present application provide liposomal compositions that are substantially free of degradation products, for example, as monitored by MALDI-TOF. In a further embodiment, the methods of the present application provide liposomal compositions that contain degradation product levels that meet the guidelines approved by the FDA or other regulatory authorities.

At this stage, the sample comprises ion-filled liposomes dispersed in a chlorite, chlorate or a mixture thereof solution. In an embodiment, the outer phase comprising the solution is replaced with, for example, saline, using an optional dialysis treatment. In an embodiment, the sample is filled in a dialysis chamber and placed in a saline solution at a temperature below the PPT of all of the one or more lipids. After about 1 hour, with stirring, the excess medium is exchanged and the procedure is repeated at least 1, 2, 3 or 4 times. In an embodiment, the procedure is repeated until the concentration of chlorite, chlorate or mixture thereof is below the detection limit of the o-tolidin method. This method for chlorite/chlorate analysis is based on the fact, that chlorite and chlorate (ClO₂⁻ and ClO₃⁻, respectively), react with ortho-tolidin (o-tolidin) in strong hydrochloric acid solutions to yield a change in color highly dependent on the amount of ClO₂⁻ and ClO₃⁻ that is present in the solution. This method provides a two-step approach to detect both species separately. However, due to the constant ratio of chlorite to chlorate in WF10, only one step needs to be used in which both constituents contributed to the change of color. Methods for performing the o-tolidin method are described in Example 7.

In an embodiment of the application, the liposomes comprising chlorite, chlorate or a mixture thereof and compositions comprising these liposomes are stored at a temperature below about 10, 9, 8, 7, or 6° C., suitably below about 6° C.

In a further embodiment, the liposome compositions may be sterilized. Non-limiting examples of suitable sterilization techniques include filtration, autoclaving, gamma radiation, and lyophilization (freeze-drying). In one embodiment, the sterilization is performed by filtration using a 220 nm filter.

### IV. Therapeutic Applications

Chlorite and chlorate are well known to be strong oxidizing agents. The oxidative power of these substances tends to increase as the pH decreases. Commercial stabilized chlorite formulations for medical use, such as WF10 and Oxoferin (a treatment for chronic wounds), are therefore generally provided as high pH aqueous solutions which contain little or no organic matter that might either react with the chlorite or decompose due to the high pH environment (e.g. due to hydrolysis of ester groups). This strategy can be employed to ensure that the products have commercially-acceptable shelf lives, but can be disadvantageous with respect to product side effects and constrain the types of products that can be developed.

For example, administering a highly basic solution, such as WF10, by intravenous infusion can result in phlebitis (inflammation of the vein) unless the infusion is performed slowly, and only after WF10 is diluted using about 250-500 mL of saline. For this reason a WF10 infusion might typically be administered over a period such as 1.5 hours resulting in patient inconvenience and significant utilization of medical resources. Similarly, Oxoferin is approved in several countries for the treatment of wounds and, unsurprisingly given its high pH, one of the most frequently observed adverse reactions is pain (See Hinz J, Hautzinger H, Stahl K W. Rationale for and results from a randomised, double-blind trial of tetrachlorodecaoxygen anion complex in wound healing. Lancet. 1986 Apr. 12; 1(8485):825-8). It may also be expected that the highly basic nature of the WF10 and Oxoferin also renders them unsuitable for other sites of administration such as to the delicate epithelium of the alveoli targeted by the pulmonary mode of delivery.

It will be also appreciated by those skilled in the art of pharmaceutical dosage form development that the difficulty of co-formulating chlorite, chlorate or mixture thereof with many organic materials can impose undesirable limitations. For example some dressing materials do not show long-term stability when impregnated with Oxoferin making it challenging to develop a product such as an individually pouched wound healing dressing which is preloaded with Oxoferin. It is also common practice to develop drug products containing more than one active pharmaceutical ingredient and it would be anticipated that many actives will not show commercially-acceptable stability if admixed with solutions containing chlorite, chlorate or mixture thereof.

WF10 is believed to exert its therapeutic benefit by its action on macrophages (see McGrath, Kodelja, V. Balanced macrophage activation hypothesis: a biological model for development of drugs targeted at macrophage functional states. Pathobiology, 67, 277-281 (1999)). A further aspect of WF10 is that it is often administered at a dose which is quite close to the limit at which toxic side effects may appear. For example in a trial of WF10 on patients with advanced AIDS, a WF10 dose of 0.5 mL/kg was employed because a previous dose ranging trial which had used doses ranging from 0.1 mL/kg to 1.5 mL/kg, had shown phlebitis, increases in methemoglobin levels, and reductions in red blood cell (RBC) glutathione reductase levels in patients receiving greater than 0.5 ml/kg which was, therefore, considered to be the maximum tolerated dose (see Raffanti, S. P., Schaffner, W., Federspiel, C. F., Blackwell, R. B., Ah Ching, 0., Kühne, F. W. Randomized, double blind, placebo-controlled trial of the immune modulator WF10 in patients with abvanced AIDS. Infection, Vol. 26, 4, 201-206 (1998)).

The effects of OXO-K993 (in the form of WF10) have also been studied on the healing and prevention of complications caused by radiotherapy on patients with advanced collum carcinoma (see Rattka, P., Hinz, J. Influence of TCDO on the results of radiotherapy in advanced collum carcinoma stage IIIB. Abstract: Int. Symp. on Tissue Repair, Pattaya, Thailand, Dec. 6-8 (1990) in: Highlights—International Symposium on Tissue Repair, 30-31 (1990)). In this study WF10 was administered 1.5 hours prior to irradiation to patients in the treatment arm and the authors of the paper developed the impression that WF10 treatment given before irradiation significantly increase biological effects on tumor tissue during radiation. For such treatments it would be desirable to have a means for targeting a stabilized chlorite formulation to the tumor tissue, although unfortunately this is difficult with a solution formulation of stabilized chlorite such as WF10 administered intravenously.

The ability to encapsulate chlorite, chlorate or a mixture thereof in liposomes in a formulation in which the external phase is substantially free of these agents has a number of advantages. For example, the external phase of the composition containing liposomes may also contain matter such as organic substances which would not be compatible with chlorite, chlorate or a mixture thereof including for example certain active pharmaceutical ingredients and excipients. The liposomal formulation may also be dispersed in a solid matrix that would not otherwise be readily compatible with these agents such as a dressing material for application to wounds. Similarly the ability to create a chlorite- or chlorate-free external phase of approximately neutral pH can be advantageously exploited to reduce the occurrence of side effects, such as phlebitis and pain, observed when high pH solutions are administered to the body intravenously or topically and to enable chlorite, chlorate or a mixture thereof to be delivered via new modes of administration such as the pulmonary route.

It will also be appreciated that liposomal structures can be designed to be preferentially cleared from the circulation by macrophages, believed to be the site of action of chlorite or chlorate-based drugs. For example, the liposomes can lower the dose of chlorite, chlorate or mixture thereof necessary to achieve a given treatment effect and can improve the margin of safety of the agents. Alternatively, the liposomes can be used to increase the pharmacological effect of chlorite, chlorate or mixture thereof without inducing unacceptable toxicities.

Alternatively, the liposomes can be designed to be delivered to targets other than macrophages. For example, a technology such as is used by the chemotherapeutic drug Doxil (doxorubicin HCl liposome injection, Centocor Ortho Biotech Products LP) may be employed to target tumors in the body of a patient. Such targeting may be beneficially employed when it is desired to use chlorite, chlorate or a mixture thereof as a sensitizer prior to radiotherapy.

The present application therefore further includes all uses of the herein described liposomes and compositions comprising the same as well as methods which include these entities. In a particular embodiment, there is included a use of the liposomes or compositions of the present application as medicaments.

The benefits of active agents can be improved by incorporation into liposomes. Improved characteristics of liposomal compositions include, for example, altered pharmacokinetics, biodistribution and/or protection of the chlorite, chlorate or a mixture thereof. It is also possible to reduce the frequency of administration of the agents or to provide a more rapid infusion rate by providing sustained release formulations. Targeted delivery of chlorite, chlorate or a mixture thereof into macrophages in the lung or to inflamed tissue is also possible with the liposomal compositions of the present application. By targeting delivery to a target site, it is possible to reduce dosing, for example, by a factor of about 1 to about 1000. Benefits may also include reducing the occurrence of side effects, such as phlebitis and pain, observed when high pH chlorite solutions are administered to the body intravenously or topically and to enable agents to be delivered via new modes of administration such as the pulmonary route or bolus injection (e.g. composition delivered via I.V. during a short period of time, e.g. less than 30 min., less than 15 min., less than 5 min.), which typically requires lower dose volumes.

One particular benefit of the liposomes of the present application is the ability to adjust the timing of the release of the encapsulated agent by varying the composition of the lipid membrane. Accordingly, the timing of the release of an encapsulated agent or agents, may be controlled by selecting a combination of lipids that have a desired temperature release profile. For example, for wound healing applications, the lipids may be selected such that the release temperature is at or near skin temperature and administration of the liposomes to the skin causes minimal or gradual release as the composition reaches skin temperature, at which time the bulk of the agent is released.

Accordingly, the application further includes a method for treating a disease, disorder or condition for which administration of chlorite, chlorate or a mixture thereof is beneficial comprising administering an effective amount of a composition of the application to a subject in need thereof.

The application further includes a use of a composition of the application for treating a disease, disorder or condition for which administration of chlorite, chlorate or a mixture thereof is beneficial and a composition of the application for use to treat a disease, disorder or condition for which administration of chlorite, chlorate or a mixture thereof is beneficial.

In one embodiment, the present application includes a method for regulating macrophage function comprising administering an effective amount of a composition of the application to a subject in need thereof. The application further includes a use of a composition of the application for regulating macrophage function and a composition of the application for use to regulate macrophage function.

Without being bound by theory, regulating macrophage function has been associated with treatment of diseases that produce symptoms of chronic inflammation as a result of an inappropriate immune response (see, for example, U.S. patent application publication no. 2011/0076344). Accordingly, in an embodiment, the diseases, disorders or conditions for which administration of chlorite, chlorate or a mixture thereof is beneficial are selected from those derivable from an antigen-specific immune responses, including, for example, autoimmune diseases and diseases caused by inappropriate immune response such as myasthenia gravis, systemic lupus erythematosus, serum disease, diabetes, rheumatoid arthritis, juvenile rheumatoid arthritis, rheumatic fever, Sjorgen syndrome, systemic sclerosis, spondylarthropathies, Lyme disease, sarcoidosis, autoimmune hemolysis, autoimmune hepatitis, autoimmune neutropenia, autoimmune polyglandular disease, autoimmune thyroid disease, multiple sclerosis, inflammatory bowel disease, colitis, Crohn's disease, chronic fatigue syndrome, and the like. Chronic obstructive pulmonary disease (COPD) also may have some autoimmune etiology, at least in some patients. In an autoimmune response, the patient's body produces too many cytotoxic T-lymphocytes (CTLs), or other cytokines which turn against the body's own healthy cells and destroy them. In transplant or graft patients, an inappropriate immune response occurs because the immune system recognizes the transplanted organ or graft's antigens as foreign, and hence, destroys them. This results in graft rejection. Likewise, transplant and graft patients can develop a graft vs. host response where the transplanted organ or graft's immune system recognizes the host's antigen as foreign and destroys them. This results in graft vs. host disease. Other inappropriate immune responses are observed in allergic asthma, allergic rhinitis and atopic dermatitis. In addition, diseases that produce symptoms of chronic inflammation also involve an inappropriate immune response, characterized by excessive macrophage activation. For example, a healthy response to tissue insult, such as a physical wound, or invasion by pathogenic organisms such as bacteria or viruses, involves activation of macrophages (via the “conventional,” proinflammatory route) and leads to an inflammatory response. However, this response can “overshoot” in an inappropriate manner, leading to chronic inflammation if the proinflammatory immune response cannot be suppressed. Diseases such as hepatitis B and C, chronic hepatitis, and manifestations of COPD such as obstructive bronchitis and emphysema that apparently are caused by prolonged exposure to non-specific bronchial and pulmonary irritants, are characterized by chronic inflammation (of the liver in hepatitis and of the pulmonary tissue in COPD) induced by excessive macrophage activation.

Other diseases, disorders or conditions for which administration of chlorite, chlorate or a mixture thereof is beneficial are selected from neoplastic disorders (cancer), HIV infection, AIDS, neurodegenerative disease, AIDS-associated dementia, stroke, spinal cord pathology, microbial infections and other viral infections.

Examples of neurodegenerative diseases include, for example, amyotrophic lateral sclerosis (ALS), Alzheimer's disease (AD), Parkinson's disease (PD) and multiple sclerosis (MS). The cancer can be, without limitation, adrenal cortical cancer, anal cancer, aplastic anemia, bile duct cancer, bladder cancer, bone cancer, bone metastasis, central nervous system (CNS) cancers, peripheral nervous system (PNS) cancers, breast cancer, Castleman's Disease, cervical cancer, childhood Non-Hodgkin's lymphoma, colon and rectum cancer, endometrial cancer, esophagus cancer, Ewing's family of tumors (e.g., Ewing's sarcoma), eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine cancer (e.g., uterine sarcoma), vaginal cancer, vulvar cancer, and Waldenstrom's macroglobulinemia.

In an embodiment, the disease, disorder or condition for which administration of chlorite, chlorate or a mixture thereof is beneficial is selected from allergic asthma, allergic rhinitis, atopic dermatitis, neoplastic disorders, HIV infection, and AIDS. In an embodiment, the neoplastic disorder or cancer is a cancer of the gastrointestinal tract, head, neck, breast or pancreas.

Other therapeutic applications for which administration of chlorite, chlorate or a mixture thereof is beneficial include treatment of patients suffering from radiation syndrome or exposure to environmental toxins. The radiation syndrome may include an acute radiation syndrome or a delayed effect of radiation exposure caused by radiation therapy or, for example, a nuclear weapon used in warfare or a terrorist attack. Therapeutic applications for wound healing, including pressure, post-operative or post-traumatic wound healing, or chronic wound healing as in the healing of diabetic ulcers, venous ulcers, arterial ulcers or decubitus ulcers are also contemplated.

The compositions, and formulations thereof, of the application can be administered to a subject using any suitable route, for example, intravenous administration, intraarterial administration, intramuscular administration, intraperitoneal administration, subcutaneous administration, intradermal administration, intraarticular administration, intrathecal administration, intracerebroventricular administration, rectal administration, ocular administration, as a nasal spray, via pulmonary inhalation, and oral administration, as well as other suitable routes of administration known to those skilled in the art. Tissues which can be treated using the methods and uses of the present application include, but are not limited to, nasal, pulmonary, liver, kidney, bone, pancreas, reproductive, soft tissue, muscle, adrenal tissue and breast. Tissues that can be treated include both cancerous tissue, otherwise diseased or compromised tissue, as well as healthy tissue if so desired.

For greater certainty, the term “composition(s) of the application” refer to the liposomal compositions described herein, either as prepared or in combination with additional carriers and/or other ingredients.

The compositions, and formulations thereof, of the application, are used alone or in conjunction with (e.g., prior to, concurrently with, or after) other modes of treatments (e.g., adjunctive cancer therapy, combined modality treatments). For example, in combination with other therapeutic agents (e.g., cancer chemotherapeutic agents as described herein and known to those of skill in the art (e.g., alkylating agents, antimetabolites, taxanes, metabolic antagonist, antitumour antibiotic, plant alkaloids, hormone therapy drug, molecular target drug, etc.)), surgery, and/or radiation therapy. Where the condition being treated is cancer, the compositions and formulations thereof, described herein can be administered in conjunction with one or more of other anticancer agents or cytotoxic compounds as described herein and as known in the art, one or more additional agents to reduce the occurrence and/or severity of adverse reactions and/or clinical manifestations thereof, surgery (e.g., to remove a tumor or lymph nodes, etc.) or radiation. Where one or more of surgery or radiation are part of the treatment regimen, the compositions or formulations thereof may be administered before, concurrently, or after the radiation therapy or surgery. Likewise, the compositions, and formulations thereof, as described herein may be administered before, concurrently, or after the administration of one or more anticancer agents. The compositions and formulations thereof described herein may also be administered in conjunction with (e.g., prior to, concurrently with, or after) drugs to alleviate the symptoms associated with the condition or the treatment regimen (e.g., drugs to reduce vomiting, hair loss, immunosuppression, diarrhea, rash, sensory disturbance, anemia, fatigue, stomatitis, hand foot syndrome, etc.). The compositions may also be administered at more than one stage of (including throughout) the treatment regimen (e.g., after surgery and concurrently with and after radiation therapy, etc.). In one embodiment, the compositions, or formulations thereof, of the application are administered in combination with one or both of 5-fluorouracil and a prodrug thereof, such as capecitabine.

In a further embodiment of the application, when the compositions, and formulations thereof, are used to treat allergic asthma or allergic rhinitis, or other pulmonary or respiratory disease, disorder or condition, they are normally administered by the intravenous route or transmucosally and, more particularly, nasally, ocularly or pulmonarily. For example, compositions of the application are administered by way of a nasal spray, nasal drops and/or eye drops. It is also possible to administer compositions of the application as a fine mist to the lungs by nebulization, using any electronic or pneumatic nebulizers. For nasal administration, any state-of-the-art device suitable for producing sprays of aqueous liposomal compositions may be used. The compositions, and formulations thereof, of the application, are used alone or in conjunction with (e.g., prior to, concurrently with, or after) other modes of treatments for allergic asthma or allergic rhinitis. For example, in combination with other therapeutic agents (e.g., steroids and antihistamines) as known to those of skill in the art. The compositions of the application may also be buffered or diluted prior to use, for example, with a suitable diluent (e.g., saline) prior to administration by intravenous infusion.

In a further embodiment of the application, when the compositions, and formulations thereof, are used to treat atopic dermatitis, or other skin disease, disorder or condition, they are normally administered topically or transdermally, for example, in the form of lotions, liniments, jellies, ointments, creams, pastes, gels, hydrogels, aerosols, sprays, powders, granules, granulates, lozenges, suppositories, salve, chewing gum, pastilles, sachets, mouthwashes, tablets, dental floss, plasters, bandages, sheets, foams, films, sponges, dressings, drenches, bioadsorbable patches, sticks, and the like. The compositions, and formulations thereof, of the application, are used alone or in conjunction with (e.g., prior to, concurrently with, or after) other modes of treatments for atopic dermititis. For example, in combination with other therapeutic agents as known to those of skill in the art

### V. Formulations and Dosing

As noted previously, the compositions and pharmaceutical formulations of the application are administered to subjects in need thereof for the treatment of conditions as described herein in conjunction with the methods of use described herein.

The liposomal composition can be administered per se or as a pharmaceutical composition or formulation. Accordingly, the present application also includes pharmaceutical compositions comprising chlorite, chlorate or a mixture thereof encapsulated liposomes admixed with at least one physiologically acceptable carrier or excipient. In one embodiment, the pharmaceutical composition provides sustained release of chlorite, chlorate or a mixture thereof and therefore comprises a sustained release formulation.

As noted above, the liposomal compositions or pharmaceutical compositions or formulation thereof are administered to a subject using any suitable route, for example, intravenous administration, intraarterial administration, intramuscular administration, intraperitoneal administration, subcutaneous administration, intradermal administration, transdermal administration, epicutaneous administration, intraarticular administration, intrathecal administration, intracerebroventricular administration, as a nasal spray, via pulmonary inhalation, and oral administration, as well as other suitable routes of administration known to those skilled in the art, and are formulated accordingly.

Depending on the mode of administration, the pharmaceutical compositions may be in the form of liquid, solid, or semi-solid dosage preparation. For example, the compositions may be formulated as solutions, dispersion, suspensions, emulsions, mixtures, lotions, liniments, jellies, ointments, creams, pastes (including toothpastes), gels, hydrogels, aerosols, sprays (including mouth sprays), powders (including tooth powders), granules, granulates, lozenges, salve, chewing gum, pastilles, sachets, mouthwashes, tablets, dental floss, plasters, bandages, sheets, foams, films, sponges, dressings, drenches, bioadsorbable patches, sticks, tablets, buccal tablets, troches, capsules, elixirs, suspensions, syrups, wafers, modified release tablets, and the like.

The pharmaceutical compositions of the present application may be formulated according to general pharmaceutical practice (see, for example, Remington's Pharmaceutical Sciences (2000-20th edition) and in The United States Pharmacopeia The National Formulary (USP 34 NF19)).

Physiologically acceptable carriers or excipients for use with the pharmaceutical compositions of the application can be routinely selected for a particular use by those skilled in the art. These include, but are not limited to, solvents, buffering agents, inert diluents or fillers, suspending agents, dispersing or wetting agents, preservatives, stabilizers, chelating agents, emulsifying agents, anti-foaming agents, gel-forming agents, ointment bases, penetration enhancers, humectants, emollients, and skin protecting agents.

Examples of solvents are water, alcohols, vegetable, marine and mineral oils, polyethylene glycols, propylene glycols, glycerol, and liquid polyalkylsiloxanes. Inert diluents or fillers may be sucrose, sorbitol, sugar, mannitol, microcrystalline cellulose, starches, calcium carbonate, sodium chloride, lactose, calcium phosphate, calcium sulfate, or sodium phosphate. Examples of buffering agents include citric acid, acetic acid, lactic acid, hydrogenophosphoric acid, and diethylamine. Suitable suspending agents are, for example, naturally occurring gums (e.g., acacia, arabic, xanthan, and tragacanth gum), celluloses (e.g., carboxymethyl-, hydroxyethyl-, hydroxypropyl-, and hydroxypropylmethyl-cellulose), alginates and chitosans. Examples of dispersing or wetting agents are naturally occurring phosphatides (e.g., lecithin or soybean lecithin), condensation products of ethylene oxide with fatty acids or with long chain aliphatic alcohols (e.g., polyoxyethylene stearate, polyoxyethylene sorbitol monooleate, and polyoxyethylene sorbitan monooleate).

Preservatives may be added to a pharmaceutical composition of the application to prevent microbial contamination that can affect the stability of the formulation and cause infection in the patient. Suitable examples of preservatives include parabens (such as methyl, ethyl, propyl, p-hydroxybenzoate, butyl, isobutyl, and isopropylparaben), potassium sorbate, sorbic acid, benzoic acid, methyl benzoate, phenoxyethanol, bronopol, bronidox, MDM hydantoin, iodopropynyl butylcarbamate, benzalconium chloride, cetrimide, and benzylalcohol. Examples of chelating agents include sodium EDTA and citric acid.

Examples of emulsifying agents are naturally occurring gums, naturally occurring phosphatides (e.g., soybean lecithin; sorbitan mono-oleate derivatives), sorbitan esters, monoglycerides, fatty alcohols, and fatty acid esters (e.g., triglycerides of fatty acids). Anti-foaming agents usually facilitate manufacture, they dissipate foam by destabilizing the air-liquid interface and allow liquid to drain away from air pockets. Examples of anti-foaming agents include simethicone, dimethicone, ethanol, and ether.

Examples of gel bases or viscosity-increasing agents are liquid paraffin, polyethylene, fatty oils, colloidal silica or aluminum, glycerol, propylene glycol, carboxyvinyl polymers, magnesium-aluminum silicates, hydrophilic polymers (such as, for example, starch or cellulose derivatives), water-swellable hydrocolloids, carrageenans, hyaluronates, and alginates. Ointment bases suitable for use in the compositions of the present application may be hydrophobic or hydrophilic, and include paraffin, lanolin, liquid polyalkylsiloxanes, cetanol, cetyl palmitate, vegetable oils, sorbitan esters of fatty acids, polyethylene glycols, and condensation products between sorbitan esters of fatty acids, ethylene oxide (e.g., polyoxyethylene sorbitan monooleate), and polysorbates.

Examples of humectants are ethanol, isopropanol glycerin, propylene glycol, sorbitol, lactic acid, and urea. Suitable emollients include cholesterol and glycerol. Examples of skin protectants include vitamin E, allatoin, glycerin, zinc oxide, vitamins, and sunscreen agents.

In an embodiment the compositions of the application are stored at a temperature below about 10, 9, 8, 7, or 6° C., suitably below about 6° C.

In a further embodiment, the compositions of the present application are lyophilized or freeze-dried. Techniques for liposome lyophilization are well known, for example, Chen et al. (J. Control Release 2010 Mar. 19; 142(3):299-311) summarizes key factors determining the lyoprotective effect of freeze-dried liposomes.

The compositions of the application, will generally be used in an amount effective to achieve the intended result, for example in an amount effective to treat or prevent the particular condition, disease or disorder being treated. The dose and/or ratio of chlorite, chlorate or a mixture thereof administered to the subject using the compositions and liposomes of the application is readily determined by those of skill in the art. In one embodiment, administration of the effective amount of chlorite, chlorate or a mixture thereof encapsulated in the liposomal formulation to treat a disease or disorder is reduced by a factor of about 1 to about 1000 compared to the effective amount of chlorite, chlorate or a mixture thereof that is administered using standard dosing regimens to treat the same disease or disorder.

In one embodiment, the compositions, or formulations thereof, of the application are administered intravenously over an extended time period, for example over about 1 minute to several hours, for example, 2, 3, 4, 6, 24 or more hours.

In one embodiment, the treatment is administered once a day. In another embodiment, the treatment is administered twice a day. In still another embodiment, the treatment is administered three times a day. In yet another embodiment, the treatment is administered four times a day. In a further embodiment, the treatment is administered one to two times a day for one, two, three, four, five, six or seven days. In still a further embodiment, the treatment is administered at least once a day for a longer term such as 1, 2, 3, 4, 5, 6, 7, 8, 9, 10, 11 or 12 weeks. In an even further embodiment, the treatment is administered at least once a day until the condition has ameliorated to where further treatment is not necessary. In an alternate embodiment, the treatment provides sustained release of the active and administration is require less frequently, for example, once a week, once a month, once every 6 months, once every year, once every two years, or once every five years. In another embodiment, the persistence of the disease or disorder is reduced for a period of time following administration of the liposomal composition, for example, for six months, one year or two years.

In another embodiment, the treatment is administered at least once per week. In another embodiment, the treatment is administered twice per week. In still another embodiment, the treatment is administered three times per week. In yet another embodiment, the treatment is administered four times per week. In yet another embodiment, the treatment is administered five times per week. In yet another embodiment, the treatment is administered six times per week. In a further embodiment, the treatment is administered one to six times per week for one, two, three, four, five, six or seven weeks. In still a further embodiment, the treatment is administered at least once per week for a longer term such as 1, 2, 3, 4, 5, 6, 7, 8, 9, 10, 11 or 12 weeks. In an even further embodiment, the treatment is administered at least once per week until the condition has ameliorated to where further treatment is not necessary.

In another embodiment the treatment may be administered as a continuous, intermittent or patient-controlled infusion using an infusion pump. In one embodiment an infusion pump is used to administer the treatment intravenously.

### VI. Examples

### Example 1

### Preparation Sphingomyelin/WF10 Liposomes

2700 μl sphingomyelin (chicken egg SM (MW 703.3), Avanti Polar Lipids, Alabama, USA) solution (25 mg/mL solution in chloroform=67.5 mg, 0.095 mmol SM) was added to a round-bottom flask (typically 25 mL) filled ⅓ with chloroform. The solution was evaporated to dryness to provide a thin, clearly visible lipid film on the inner surface of the flask.

4800 μL of WF10 (0.095 mmol/4.8 mL) was added and the flask was shaken in a vortex mixer at approximately 45° C. (above the phase transition temperature of the lipid). The vortexing was stopped when the film was not visible anymore. The rehydration of the lipids in this step results in the formation of liposomes that are large multilamellar vesicles (LMV). WF10 is both inside (inner phase) and outside the liposomes (outer phase).

To reduce the size of the primary liposomes, the mixture was frozen and thawed repeatedly. The freeze/thaw cycle was repeated 10 times using liquid nitrogen as refrigerant and thawing in a water bath typically at 45° C. with an overall duration of about 30 to 40 min. Freezing results in a destruction of the primary liposomes. Thawing was done above the phase transition temperature. In this case, liposomes smaller than the primary liposomes form again spontaneously. All samples were well above room temperature for about 20 minutes due to the given temperature scheme (−180°/45° C.).

The above mixture (approx. 5 mL) was then extruded repeatedly through (the same) disc filter with 100 nm pore width. A stainless steel chamber with heating jacket and a 100 nm disc filter, laid double was used (although a single filter can also be used). The working temperature was 45° C. for SM. Pressurization was achieved using nitrogen at 30 bar and the extrusion was repeated 10 times which is known to result in unilamellar vesicles of a relatively uniform diameter. The inclusion rate was at approximately 2 to 3%; theoretically, 7% was possible.

The extruded mixture (outer phase: WF10, inner phase: WF10) was split up into 3 dialysis chambers. The chambers were placed floating in 0.9% sodium chloride solution. The dialysis chamber were “Slide-A-Lyzer Dialysis Cassettes, 20K MWCO”, having a 3 mL maximum volume, permeable up to 20 kDa. The liposome mixture (1 mL) was placed in the inner chamber and NaCl 0.9% (saline, 1 L) was the outer medium. The process was repeated 4 times with fresh saline. The rest of the liposome mixture (approx. 5 mL-3×1 mL dialysed) was used for phosphate determination and DSC (Differential Scanning calorimetry [symbol] determination of phase transition temperature).

At the end of the dialysis stage, the outer phase of the liposome preparation consisted of 0.9% sodium chloride solution (=saline). The purpose was to get the concentration of chlorite and chlorate in the outer phase below the detection limit of the method used for quantitation. By doing this, the leaking behaviour of the liposomes can be tested by monitoring a re-occurrence of these ions.

### Example 2

### Preparation POPC/WF10 Liposomes

Using the procedure described in Example 1, and a working temperature for the extrusion step of room temperature (heating jacket not connected), liposomes made of POPC and comprising WF10 were prepared.

### Example 3

### Preparation POPC:POPG/WF10 Liposomes

Using the procedure described in Example 1, and a working temperature for freeze/thaw of 35° C. and for the extrusion step of 23° C., liposomes made of 1-palmitoyl-2-oleoyl-sn-3-glycero-3-phosphocholine (POPC) and 1-palmitoyl-2-oleoyl-sn-glycero-3-phospho-(1′-rac-glycerol) (POPG) (3:1, 2:1 and 1:1) and comprising WF10 were prepared. POPG was obtained from Avanti Polar Lipids, Alabama, USA.

### Example 4

### Preparation DPPC/WF10 Liposomes

Using the procedure described in Example 1, and a working temperature for the extrusion step of 45° C., liposomes made of 1,2-dipalmitoyl-sn-glycero-3-phosphocholine (DPPC) and comprising WF10 were prepared. DPPC was obtained from Avanti Polar Lipids, Alabama, USA.

### Example 5

### Preparation DMPC/WF10 Liposomes

Using the procedure described in Example 1, and a working temperature for the extrusion step of 30° C., liposomes made of 1,2-dimyristoyl-sn-glycero-3-phosphocholine (DMPC) and comprising WF10 were prepared. DMPC was obtained from Avanti Polar Lipids, Alabama, USA.

### Example 6

### Additional Preparation Method

### (a) Evaporation of Lipids

Lipids dissolved in chloroform were added to a 100 mL round-bottom flask. In most cases the lipids were already purchased in solution with a typical concentration of about 25 mg/mL. Lipids delivered as powder were usually dissolved before addition to the flask, having a similar concentration. The total mass of added lipid was then controlled via the volume. Typical masses were 300-400 mg for 14 mL of a 40 mM sample. Afterwards, the flask was mounted in a Buchi ‘Rotavapor™ R-210’ rotational evaporator and was evacuated while rotating at 15 rpm in a water bath at 37° C. The pressure in the flask decreased to values of about 5-15 mbar during the operation. The evaporation was considered to be finished when all solvent was removed and no chloroform odor could be smelled in the flask. The result at this stage was a visible lipid film on the walls of the bottle and (depending on the amount of lipid) additionally accumulated fluffy powder at the flask-bottom.

### (b) Hydration

During the hydration procedure, lipids were suspended in aqueous medium, which was later entrapped in the final liposomes. As is known, during this process multilamellar vesicles (MLVs) are formed. First of all, the necessary amount of WF10 medium, was added to the round-bottom-flask. The volume was equivalent to the desired total sample volume (usually about 8 to 14 mL). The result was a turbid solution containing large lipid aggregates. Then, a water bath was prepared at a temperature of 5K above the lipid phase transition temperature (PTT) of the lipids, during which the flask was slightly shaken. Once the temperature in the flask reached the PTT, the suspension became a homogeneous milky-white liquid. After approximately five minutes at a temperature above the PTT, the lipid film was removed from the flask walls and no lipid powder remained at the bottom. Finally the flask was shaken with a vortex mixer for a few seconds. The still-heated sample was immediately passed to the extrusion device to start the next preparation step or was stored at refrigerator temperature until it was time to perform the next preparation step.

### (c) Extrusion

During the extrusion process, the sample was repeatedly squeezed through a 220 nm filter disk. This forced the large vesicles to reorganize and form smaller structures having fewer layers. Generally this yielded unilamellar vesicles, however, for relatively large pore sizes, such as 220 nm, it was still probable to obtain multilamellar vesicles but with only few layers. Prior to the extrusion step, the extruder was equipped with three stacked 220 nm filter discs (Millipore GPWP, diameter 25 mm) and was heated 5K above the LPTT. After filling the lipid suspension in the extrusion chamber, a pressure of 25 bar was applied to the chamber, forcing the sample through the filters. The pressure was applied via compressed nitrogen. The sample was collected at the extruder-outlet. The whole extrusion was performed at least 10 times.

### (d) Dialysis

At this stage the liposomes were in their final state but remained suspended in WF10 solution. To replace the outer WF10 medium with 0.9% saline solution, a series of successive dialysis steps was performed. The sample was first introduced in a Thermo Scientific Slide-A-Lyzer™ dialysis frame. The frame's membranes have a molecular weight cutoff of 20,000 Da and a capacity of 3-12 mL. For sample volumes larger than 6 mL, the sample was split and distributed over two such frames, both of which were placed in the same beaker. The dialysis setup included a 1 L glass beaker with 0.9% saline solution placed on a magnetic stirring device. The saline solution was usually made from deionized water and standard laboratory NaCl (min. purity 99%) and the volume was about 900-950 mL. The frames were submerged in the dialysis medium for approximately one hour, then the medium was exchanged with fresh medium for subsequent steps. Typically a small sample of the dialysis medium was taken for an “o-tolidin quick check”, which was similar to the usual o-tolidin analysis procedure. When the sample appeared dark yellow, the medium was exchanged earlier. Such a quick check was also performed to determine the end of the dialysis series. The dialysis was considered to be completed, when two successive dialysis steps showed no visible change of color during the test and after a dialysis duration of one hour. After each dialysis step, the dialysis medium was exchanged until at least 5 steps had been performed. After the last step, the samples were stored at ca. 6° C. for further usage.

### Example 7

### o-Tolidin Methods for Detection of Chlorite and Chlorate

### Method A

This o-tolidin method included adding 50 μl of a solution of 155 mg o-tolidine in a mixture of 200 mL water and 67 mL 12M hydrochloric acid to 200 μL of excess medium. This mixture was prepared twice. 250 μL of 4.8M or 12M hydrochloric acid, respectively, were added and the mixture was incubated for 5 to 10 minutes. Using hydrochloric acid of the lower concentration allowed detection of chlorite only, while using the higher concentration additionally allowed detection of chlorate. The absorption of the test solution was measured at 442 nm (4.8M HCl) or 445 nm (12M HCl), respectively. A calibration curve was established using standard dilutions prepared from untreated WF10.

### Method B

Method B is an improvement to Method A in that it allows the detection of both chlorite and chlorate. The method includes dissolving 114 mg o-tolidin in 150 mL of water and adding 50 mL of hydrochloric acid (37%) to prepare an o-tolidin solution. The o-tolidin solution was prepared at least 24 h prior to the first application and could be stored at 6° C. for a few months. Care was taken to measure all samples at the same time after initiation of the analysis procedure to avoid the influence of bleaching, that occurred immediately after the preparation of the analysis mixture. The analysis scheme was designed to analyze up to 96 samples simultaneously and times were chosen in a way to ensure those capacities. At the beginning of the analysis procedure, 400 μL of samples to be analyzed were filled into standard 2 mL reaction vessels. At time to, 100 μL of the o-tolidin solution were added to each of the reaction vessels. Ten minutes later, (t₀+10 min), 500 μL of hydrochloric acid (37%) was added. Another ten minutes later (t₀+20 min), 200 μL of a sample from each vessel was transferred to a standard 96-well plate for absorbance measurement at a wavelength of 447 nm. Those measurements were performed on an TECAN Infinite 200PRO™ microplate reader and were started 15 min later (t₀+35 min).

In the calculations, the concentration of WF10 in the storage medium was represented by an inverse dilution factor d. This quantity represented the volume of undiluted WF10 per volume of storage medium. It was therefore dimensionless with d=1 for undiluted WF10

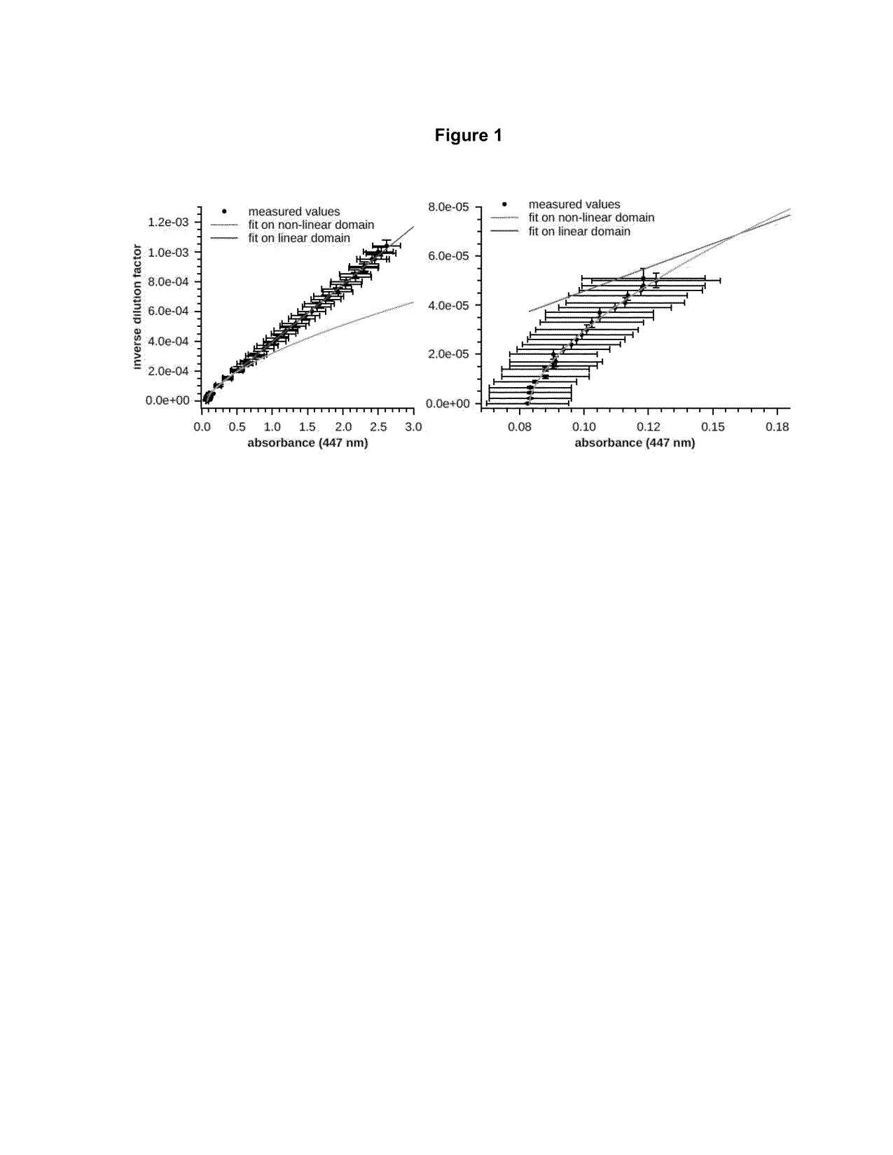

To relate the measured absorbance values to a certain inverse dilution factor, a WF10 dilution curve was recorded. The curve is shown in FIG. 1. The data showed a strong linear dependence for values of d above approximately 6×10⁻⁵ and the parameters for the corresponding equation

d(A)=mA+n (1)

have been found to be m=(3.87±0.02)×10⁻⁴ and n=(7±2)×10⁻⁶. The non-linear domain for concentrations below the mentioned value were fitted using the equation

d(A)=α|A−A₀|^{P} (2)

with A₀=0.0787±0.0007, α=(3.36±0:05)×10⁻⁴ and p=0.631±0.009. In both cases the, fits were weighted with respect to the uncertainties of the data points. To consider the relatively large uncertainties of the absorbance values, hits on the inverse dataset were carried out as well, using the inverse functions. The presented parameters were the arithmetic means for both results.

From the determined inverse WF10 dilution factor d in the storage medium, the amount, X_{WF10}, of WF10 that has been released from the liposomes' interior was calculated. Given the volume of the sample Vₛₐₘₚₗₑ as well as the volume of the storage-medium V_{storage}, the value for d was calculated using the following equation

$$d = \frac{{x}_{WF 10}}{{V}_{sample} + {V}_{storage}} \qquad ( 3 )$$

and thus

x_{WF10}=d(Vₛₐₘₚₗₑ+V_{storage}). (4)

This is, of course, only true after the leaked substance has completely mixed with the sample-medium and the storage-medium. Experiments show that this happened in a relatively short amount of time, when the appropriate setup was chosen. Problems occur when leaked substance is removed from the experimental setup, which always happens when fractions of the storage medium are taken for analysis. However, to take this removal into account, all previous measurements need to be considered in the calculation of the current dilution factor. In such an implicit determination, all errors and uncertainties accumulate with every new concentration determination, such that one single bad data point would alter all the subsequent measurements of an experiment. Therefore, an explicit approach was used, in which only d and V_{storage} of the current analysis step were considered and the substance was neglected, taken away in previous analysis steps. However, the maximum possible amount that could have been neglected was added to the uncertainties. Deviations of this simplified explicit approach from the implicit method were usually small compared to uncertainties arising from other factors. Nevertheless, alternative calculations in the course of every evaluation process were done to verify the use of the method separately for each case.

### Method C

This method is another improvement in method A in that is allows detection of chlorite but not chlorate. The o-tolidin solution was prepared at least 24 h prior to its application. Stored at 6° C. in a dark place, it was stable for several months. First, a temporary HCl-solution was prepared by adding 50 mL of concentrated (37%) hydrochloric acid to 150 mL H₂O (high-purity-water was used at this stage). Then, 58 mg of o-tolidin (dihydrochloride, H₂O content 1.5 mol/mol, 285.2 g/mol, SIGMA Prod. No.:T-6269) was flushed into a 100 mL measuring flask, using the temporary HCl-solution. The flask was filled completely (100 mL). After shaking the solution, it was filled into a brown glass bottle. After 24 h, the solution was colorless and some precipitation was found at the bottom of the flask.

4.8M hydrochloric acid was prepared immediately before the measurement. It was prepared by adding 40 mL of concentrated hydrochloric acid (37%) to 60 mL of H₂O.

The reaction causes a color change in the sample which can be detected via absorbance measurements. However, color fading starts immediately after the reaction takes place, so that time is an important parameter in this procedure. A time schedule was followed which enabled the analysis of up to 96 samples at a time. At the beginning, 400 μL of each sample was filled into 1.5 mL reaction vessels. Afterwards 100 μL of the o-tolidin solution was added to the vessels. 15 min after starting the o-tolidin addition, 500 μL of the 4.8M hydrochloric acid was given to each vessel. Then 350 μL of each sample was transferred from the reaction vessel to the cavity of a transparent 96-well plate (with cover). Another 25 min later, the photometric measurement of the samples was started by recording the absorbance at 442 nm.

Recorded absorbance values were compared to a standard curve of known concentrations, recorded under the same conditions. The dependence of the absorbance on the chlorite concentration is linear for chlorite concentrations between 2 μM and 60 μM.

### Leakage Monitoring

The basic purpose of all leakage monitoring experiments was the detection of substance which had been released from the liposome's interior. The general approach for such experiments was similar to the dialysis procedure described in Example 6(d). A substance which had been released from the liposomes in a sample passed into the surrounding sample-medium and could easily pass the dialysis membrane, such that the storage-medium, surrounding the dialysis frame, contained leaked substance at certain concentrations. For leaked chlorite/chlorate, the above o-tolidin methods are used to quantitatively or qualitatively determine leakage. From this concentration, the total amount of leaked substance could be determined, using the calculations described below. It was clear, that this concentration decreased with increasing volume of the storage solution and, thus, the storage solution was chosen to be relatively small. However to determine the concentration of leaked substance, 1 mL of the storage medium was typically required and, therefore, a storage volume of at least ten or twenty milliliters was desired.

### Example 8

### Stability of Liposomes from Examples 1-5

After the liposomes were prepared, the sample was left in the dialysis frame which was then placed in 120 mL of saline solution for storage. In intervals of 1-3 days, 1 mL of the excess medium was taken and analyzed using o-tolidin method A (Example 7).

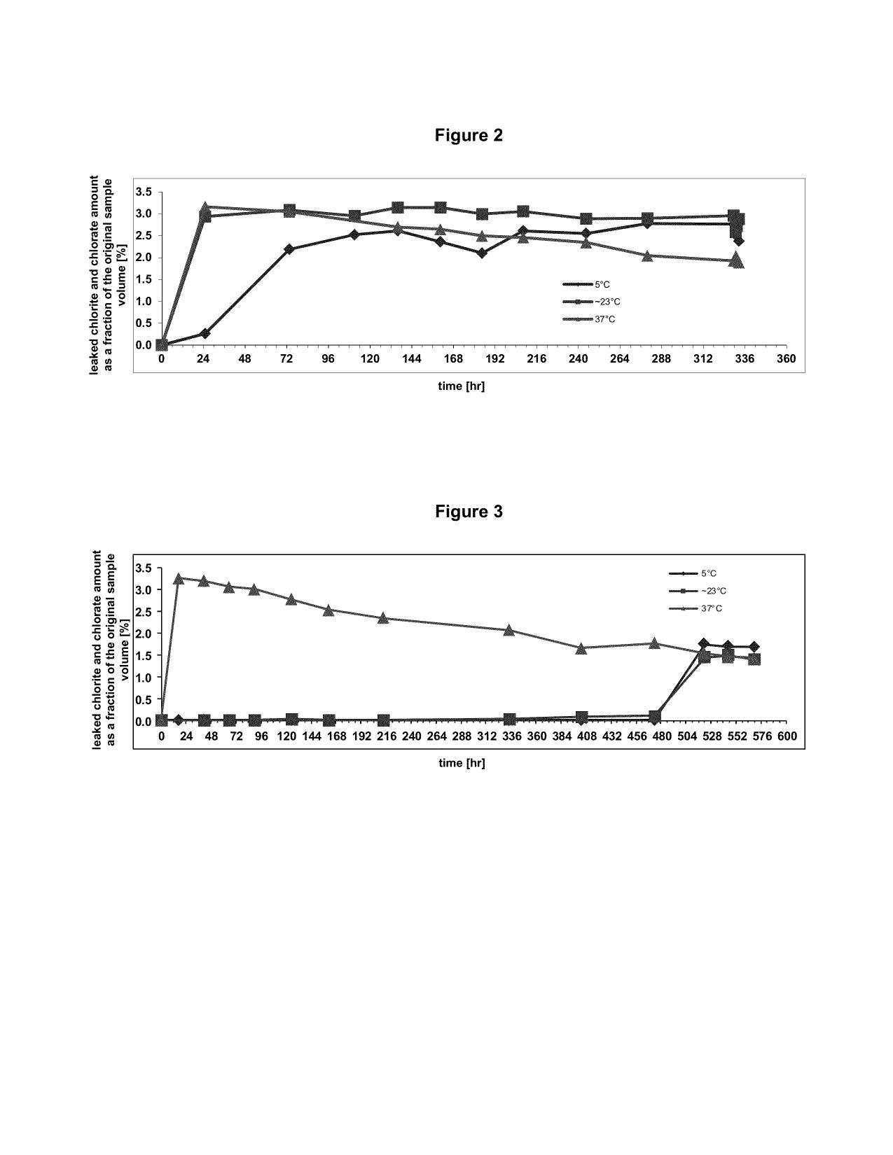

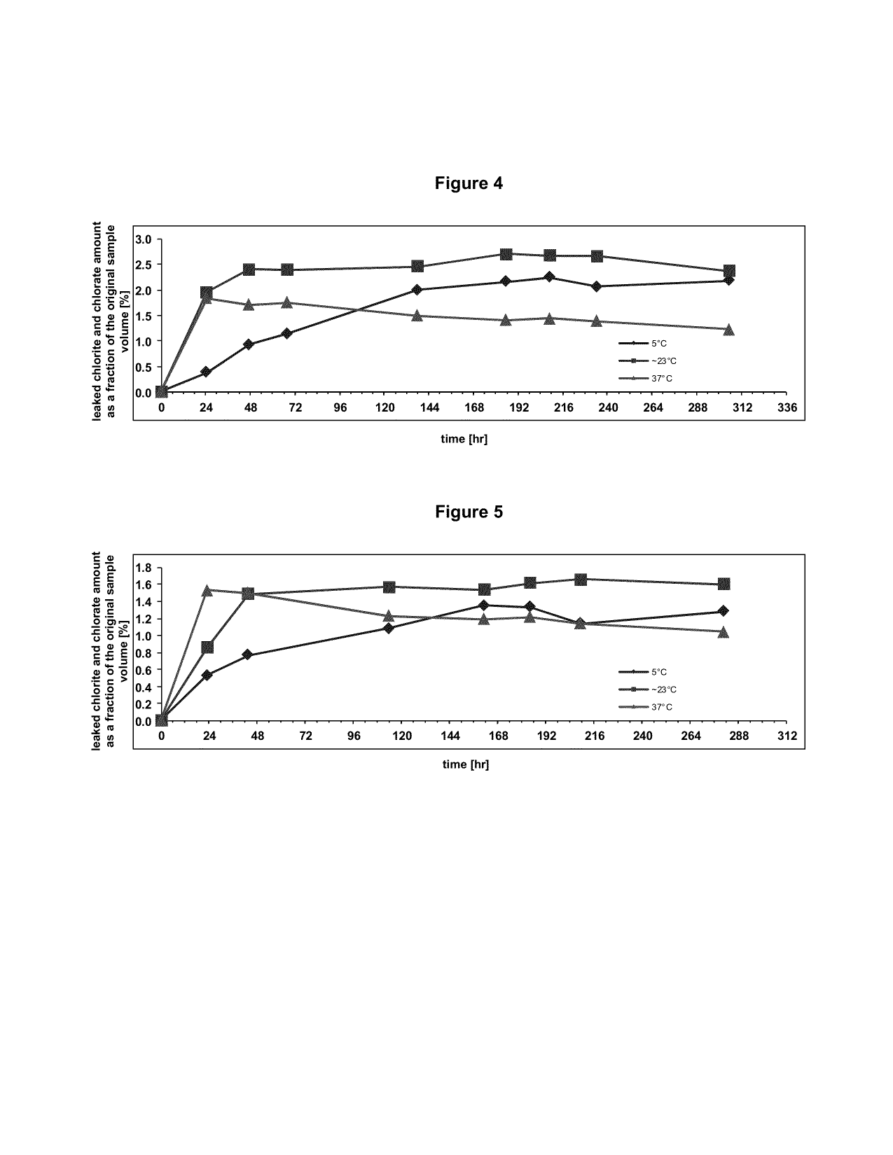

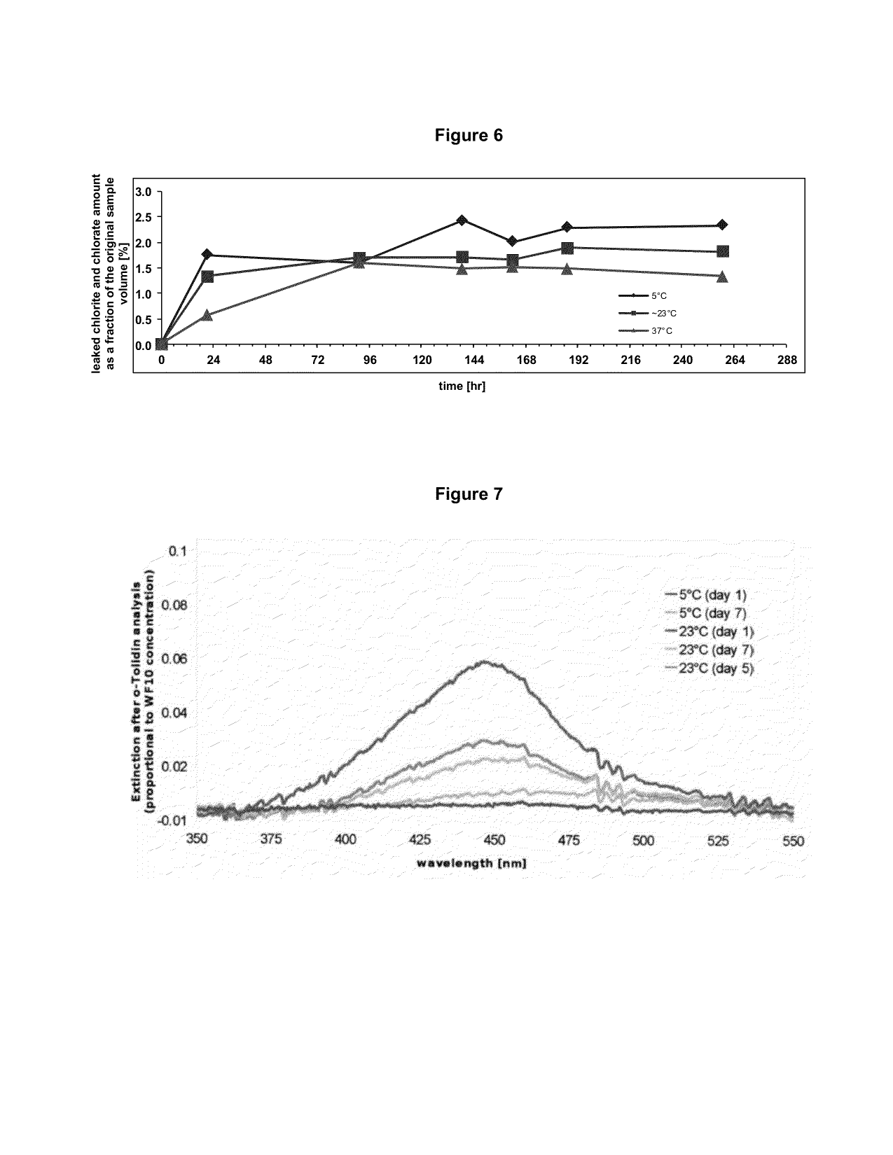

Leakage was detected from POPC liposomes at all temperatures (5° C., 23° C. and 37° C.) (see FIG. 2). In the case of SM liposomes, leakage was only detected at 37° C. (see FIG. 3). Further leakage experiments were performed on POPC/POPG liposomes as well as pure DMPC liposomes. The POPC/POPG mixtures (3:1, 2:1 and 1:1) behaved similar to the pure POPC samples and showed complete leakage after a short time (see FIGS. 4, 5 and 6, respectively). DMPC leaked near its phase transition temperature (23° C.) as shown by spectrometer readings taken at days 1, 5 and 7 from samples stored at 5° C. and 23° C. (see FIG. 7).

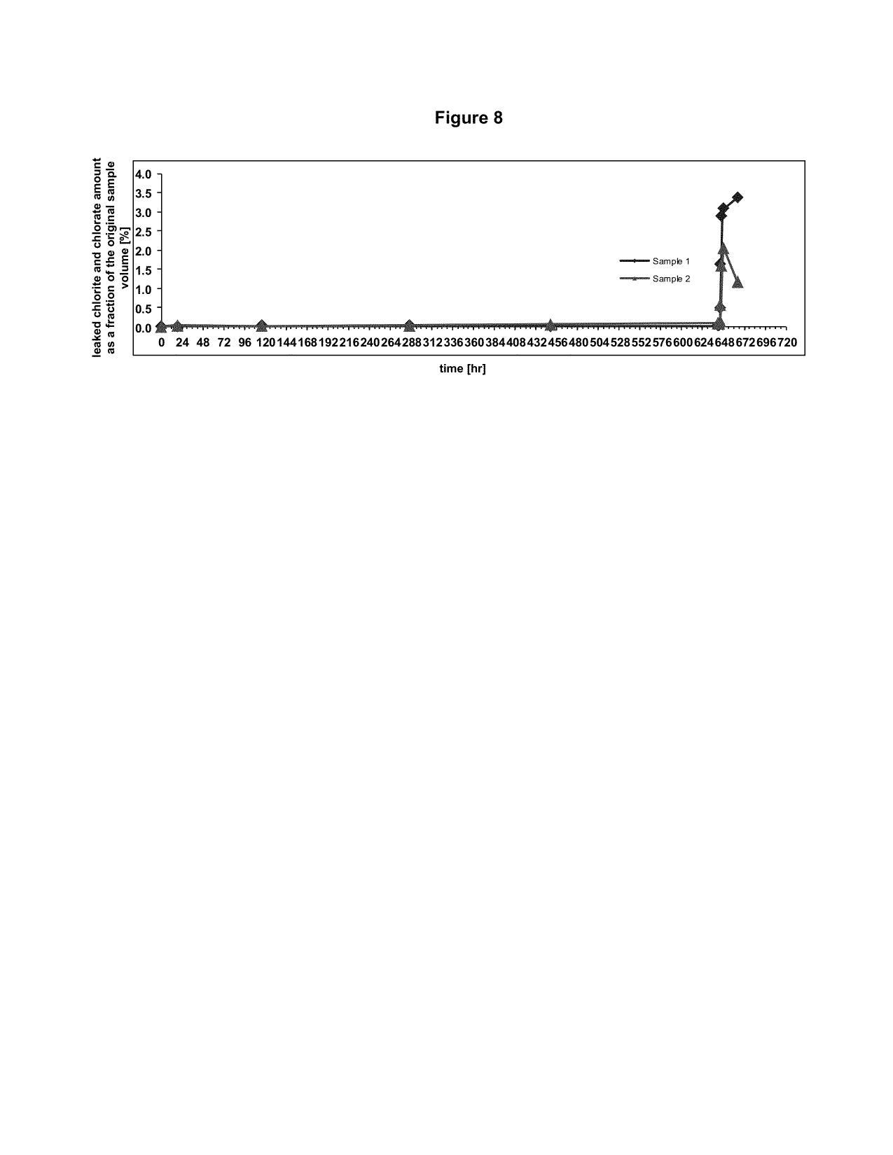

Additionally, SM experiments were repeated, yielding the same results as described above (see FIG. 8). At the end of the experiments, all samples were placed at 37° C. to monitor leakage of ions. It was found that major leakage started about 2 hours after being placed at 37° C. and was completed 4 hours after being placed at 37° C.

### Example 9

### Preparation DPPC, DPPC/DMPC, S_PC3 and S_PC3/DMPC WHO Liposomes

Using a modified version of the procedure described in Example 1 (freeze/thaw step omitted; 220 nm pore size of membrane), liposomes were made of DPPC, DPPC/DMPC, hydrogenated soybean phospholipid (S_PC3) and S_PC3/DMPC. The ingredients and extrusion temperatures are provided in Table 2. DPPC was obtained from Avanti Polar Lipids, Alabama, USA while S_PC3 and DMPC were obtained from LIPOID AG.

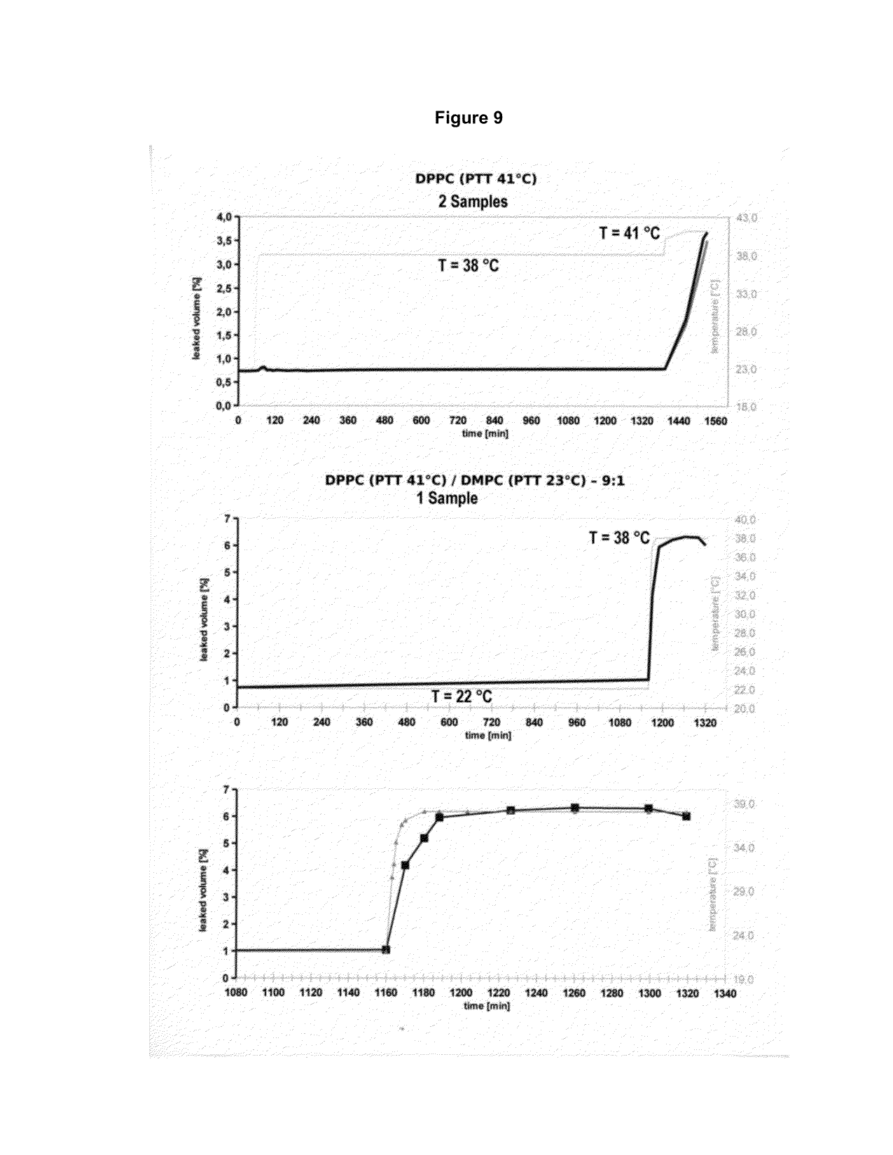

Leakage was detected from DPPC liposomes at the phase transition temperature of 41° C. for DPPC (see FIG. 9). At 38° C., 3 degrees below phase transition temperature of DPPC (41° C.), the liposomes were tight. DPPC:DMPC 9:1 leaked at 38° C., below the phase transition temperature (PTT) for DPPC. Further leakage experiments were performed on S_PC3 liposomes as well as S_PC3:DMPC 3:5 with leakage detected from S_PC3 liposomes at 60° C. (Table 2).

### Example 10

### MALDI-TOF MS Measurements to Find Possible Degradation Products

### Preparation and Experiments—Samples

Three samples were prepared using the method described in Example 6. For all samples 1,2-diapalmitoyl-sn-glycero-3-phosphocholine (DPPC) was used. The samples were named as follows:

SX123: ordinary WF10

SX124: WF10 buffered with PBS

SX125: WF10 buffered with taurine

### SX123:

9:2 mL of a 25 mg/mL DPPC-chloroform-solution was added to a round-bottom-flask. This corresponds to a lipid mass of 233 mg. DPPC was purchased from Avanti Polar Lipids (Product no.: 850355C). Hydration was done with 7:92 mL WF10 prepared from OXO-K993 25.03 g OXO-K993 in 250 mL solution. During the hydration process, the sample was heated to 46° C. for 5 min. Extrusion took place at 50° C. and lasted 19 min. 10 consecutive passes were performed. Samples were stored overnight at 6° C. in a fridge. Dialysis steps were performed in 900 mL saline solution, prepared with 45:001 g NaCl per 5000 mL solution. 5 subsequent steps lasting 22, 40, 47, 61 and 71 min were carried out, a 6^{th} step was carried out overnight in a 6° C. fridge. SX124

Preparation of “WF10/PBS”: 10 g of OXO-K993 were diluted to 80 g with water for injection (WFI). The pH was adjusted to an immediate pH of 7.36 by adding an aqueous solution of sodium dihydrogenphosphate monohydrate (4.31 g/25 mL). The mixture was completed to 100 g using WFI and contained chloride, chlorite, chlorate and sulfate in the concentrations present in WF10.

9.2 mL of a 25 mg/mL DPPC-chloroform-solution was added to a round-bottom-flask. This corresponds to a lipid mass of 233 mg. DPPC was purchased from Avanti Polar Lipids (Product no.: 850355C). Hydration was done with 7.92 mL WF10/PBS (pH 7.63 on use). During the hydration process, the sample was heated to 50° C. for 2 min. Extrusion took place at 50° C. and lasted 7 min. consecutive passes were performed. Samples were stored overnight at 6° C. in a fridge. Dialysis steps were performed in 900 mL saline solution, prepared with 45.001 g NaCl per 5000 mL solution. 5 subsequent steps lasting 22, 40, 47, 61 and 71 min were carried out, a 6^{th} step was carried out overnight in a 6° C. fridge.

### SX125

Preparation of “WF10/Taurine”: 10 g of OXO-K993 were diluted to 80 g with WFI. The pH was adjusted to an immediate pH of 8.49 using an aqueous solution of taurine (3.91 g/50 mL). The mixture was completed to 100 g using WFI and contained chloride, chlorite, chlorate and sulfate in the concentrations present in WF10.

9.2 mL of a 25 mg/mL DPPC-chloroform-solution was added to a round-bottom-fask. This corresponds to a lipid mass of 233 mg. DPPC was purchased from Avanti Polar Lipids (Product no.:850355C). Hydration was done with 7.92 mL WF10/Taurine (pH 8.47 on use). During the hydration process, the sample was heated to 50° C. for 4 min. Extrusion took place at 50° C. and lasted 7 min. 10 consecutive passes were performed. Afterward samples were stored overnight at 6° C. in a fridge. Dialysis steps were performed in 900 mL saline solution, prepared with 44.998 g NaCl per 5000 mL solution. 5 subsequent steps lasting 22, 40, 47, 61 and 71 min were carried out, a 6^{th} step was carried out overnight in a 6° C. fridge.

### Storage Experiments

On Dec. 16, 2011, the samples were removed from the dialysis frames and were stored at 6° C. until Dec. 19, 2011. On this day 2 mL of each sample was removed for storage in a separate vessel at 6° C. Another 2 mL was stored in the same way at room temperature (˜22° C.). The rest of the samples were used for different purposes. On Dec. 19, 2011, Jan. 6, 2012 and Feb. 1, 2012, 0.2 mL of sample was removed from each vessel and put in a −80° C. freezer for later MALDI-TOF measurements.

### MALDI-TOF MS

A total of 18 samples were prepared for MALDI-TOF MS measurements (3 sample types, each stored at two temperatures, all at 3 different dates) using the following procedure:

Samples were diluted prior to the preparation using 560 μL of 0.9% saline solution and 40 μL of the original sample, stored at −80° C. Afterward a lipid extraction procedure followed. Therefore, 100 μL of prediluted sample was mixed with 200 μL chloroform/methanol-mixture (1:1). The resulting mixture was then centrifuged at 800 g for 5 min, resulting in a visible phase separation. 50 μL of the lower phase were taken for further preparation. To this volume 3.7 μL of a 25 mg/mL DMPC-chloroform-solution and 2.5 μL of a 25 mg/mL 14:0-Lyso PC-chloroform-solution were added as standards. Afterwards, all solvent was removed in a vacuum centrifuge. Then, 100 μL of a DHB-Matrix (156 mg dihydroxy benzoic acid, 1998 μL, 2 μL trifluoroacetic acid) and 100 μL chloroform were added to the evaporated samples. Finally 1 μL of the prepared sample was placed on a gold-coated target under a stream of warm air.

MALDI-TOF MS measurements were carried out on an autoflex LRF MS spectrometer by Bruker Daltronics, Leipzig.

### Results and Discussion

In all 18 cases the recorded spectra indicated absolutely no sign of lyso products. The term “lyso” is used to refer to phospholipids in which one of the two O-acyl chains has been removed. Signals of the two added standards as well as the lipids used for liposome preparation were clearly identified. No other signals were found in the spectra.

Accordingly, no hydrolysis and/or other degradation processes occurred at 6° C. or room temperature, at the high pH-values of ordinary WF10 or at the lower pH-values of the alternate buffered versions.

### Example 11

### Long Term Stability Tests on DPPC and DPPC/DMPC Liposomes

### Samples

Six liposome samples were assayed. The first three samples were the DPPC-based liposome samples SX123, SX124 and SX125 prepared as described in Example 10. The remaining three samples (samples SX130, SX131 and SX132) contained liposomes that were prepared with a 9:1 (mol ratio) mixture of DPPC and DMPC as described below.

### SX130: Ordinary WF10

3.14 mL of a 84.3 mg/mL DPPC-chloroform-solution and 2.00 mL of a 13.4 mg/mL DMPC-chloroform-solution were added to a round-bottom flask. This corresponds to a lipid mass of 264 mg and 27 mg, respectively. Hydration was done with 10.00 mL WF10. During the hydration process, the sample was heated to 46±1° C. for 4 min. 10 consecutive extrusion passes followed at 46±1° C. and lasted 20 min in total. Afterwards, samples were stored overnight at 6° C. in a fridge. Dialysis steps were performed in 900 mL saline solution, prepared with 45.008 g NaCl per 5000 mL solution. 4 subsequent steps lasting 46, 65, 75, and 174 min were carried out—a 5^{th} step was carried out overnight.

SX131: WF10 Buffered with Taurine

3.14 mL of a 84.3 mg/mL DPPC-chloroform-solution and 2.00 mL of a 13.4 mg/mL DMPC-chloroform-solution were added to a round-bottom flask. This corresponds to a lipid mass of 264 mg and 27 mg, respectively. Hydration was done with 10.00 mL taurine-buffered WF10. During the hydration process, the sample was heated to 46° C. for 4 min. 10 consecutive extrusion passes followed at 46° C. and lasted 13 min in total. Afterwards, samples were stored overnight at 6° C. in a fridge. Dialysis steps were performed in 900 mL saline solution, prepared with 45.010 g NaCl per 5000 mL solution. 4 subsequent steps lasting 46, 65, 75, and 174 min were carried out—a 5^{th} step was carried out overnight.

SX132: WF10 Buffered with PBS

3.14 mL of a 84.3 mg/mL DPPC-chloroform-solution and 2.00 mL of a 13.4 mg/mL DMPC-chloroform-solution were added to a round-bottom flask. This corresponds to a lipid mass of 264 mg and 27 mg, respectively. Hydration was done with 10.00 mL PBS-buffered WF10, prepared as described in Example 10. During the hydration process, the sample was heated to 46° C. for 3 min. 10 consecutive extrusion passes followed at 46° C. and lasted 11 min in total. Afterwards, samples were stored overnight at 6° C. in a fridge. Dialysis steps were performed in 900 mL saline solution, prepared with 45.015 g NaCl per 5000±mL solution. 4 subsequent steps lasting 46, 65, 75, and 174 min were carried out—5^{th} step was carried out overnight.

### Long Term Stability (LTS) Experiments

LTS-experiments were established to monitor liposome leakage over periods of weeks or months. Therefore, the setup was designed in a way to enable a multitude of experiments running parallel. For this purpose commercially available gel staining trays with dimensions of approximately (75×110×25)mm³ were used, such that a relatively small volume of storage-medium was required to completely immerse the dialysis frame. A cover was provided to tightly seal the trays while storing them under appropriate conditions of interest. The usual LTS setup included a Slide-A-Lyzer™ dialysis frame containing 1 mL of sample, immersed in 120 mL 0.9% saline solution as storage-medium. The dialysis frame was similar to the one used during dialysis (Example 6(d)), but having a capacity of only 0.5-3.0 mL.

### Storage Experiments

After the dialysis procedure, the samples were removed from the dialysis frames and were stored at 6° C. overnight. The LTS experiments were prepared as described above, using Slide-A-Lyzer dialysis cassettes (Thermo Scientific, 0.5-3.0 mL, 20K MWCO) and 120 mL of 0.9% saline solution in standard polypropylene gel-staining trays. For each sample, 4 separate LTS setups were prepared, each having a sample volume of 1 mL. Two of those setups where stored at room temperature, while the other two were placed in a fridge at 6° C. To analyze the WF10 concentration in the outer medium and, thus, the amount of chlorate and chlorite, leaked from the liposomes, 400 μL of the outer saline medium was removed for further analysis with the o-tolidin method, as described in Example 7, Method B. The following table indicates the dates at which the analysis samples were taken.

| Date | Samples |
|---|---|
| Dec. 19, 2011 | SX123, SX124 and SX125 (start) |
| Jan. 6, 2012 | SX123, SX124 and SX125 (analysis) |
| Jan. 16, 2012 | SX123, SX124 and SX125 (analysis) |
| Mar. 7, 2012 | SX130, SX131 and SX132 (start) |
| | SX123, SX124 and SX125 (analysis) |
| Mar. 29, 2012 | analysis of all samples |
| Apr. 27, 2012 | analysis of all samples |
| May 25, 2012 | analysis of all samples |
| Jul. 6, 2012 | analysis of all 6° C. samples |

### Evaluation

Evaluation of the recorded absorption data took place according to the procedures defined in Example 7, Method B, regarding the transformation of absorbance to a WF10 dilution factor. For display purposes, WF10 dilution factors were converted to a more convenient quantity: The amount of leaked WF10 as a volume fraction of the total sample. Thus, a value of 2% leakage in a 1 mL sample means that a total of 20 μL WF10 was released from the liposomes interior. The detection and quantitation limits are defined in the subsections below. A discussion on uncertainties arising from the storage volume is also provided below.

### Limit of Detection (LOD)

A measurement value was defined as the limit of detection. The value was defined such that, above the value, the presence of chlorite/chlorate WF10 in the medium can be considered verified. The absorbance value of 0.095, which is slightly above the uncertainty range of the zero value, was used. This explicit value is only valid for measurements performed according to Example 7, method B.

### Limit of Quantitation

When calculating the amount of leaked WF10 as a fraction of the sample volume, processing data points close to the LOD often yields results with uncertainties. Therefore a second limit was defined (the limit of quantitation (LOQ)) which considered all parameters that contribute to the calculations of the final results (such as the volumes of the sample and the outer dialysis medium). Those parameters were kept constant for the whole experiment and, therefore, resulted in a single LOQ which indicates the point where the total relative uncertainty of the final result falls below 50% of its value. In the present study the amount of leaked WF10 equal to 0.61 vol. % of the sample volume was defined to be the LOQ.

### Results

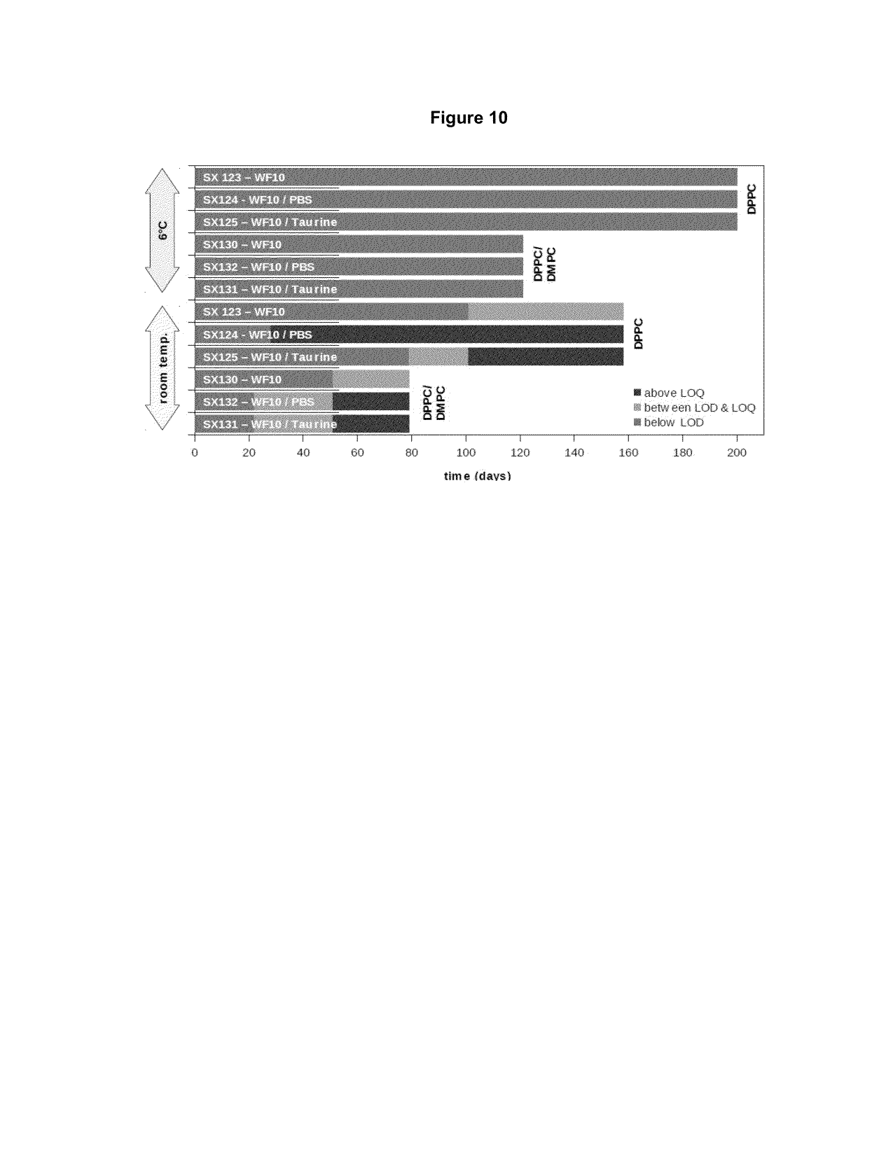

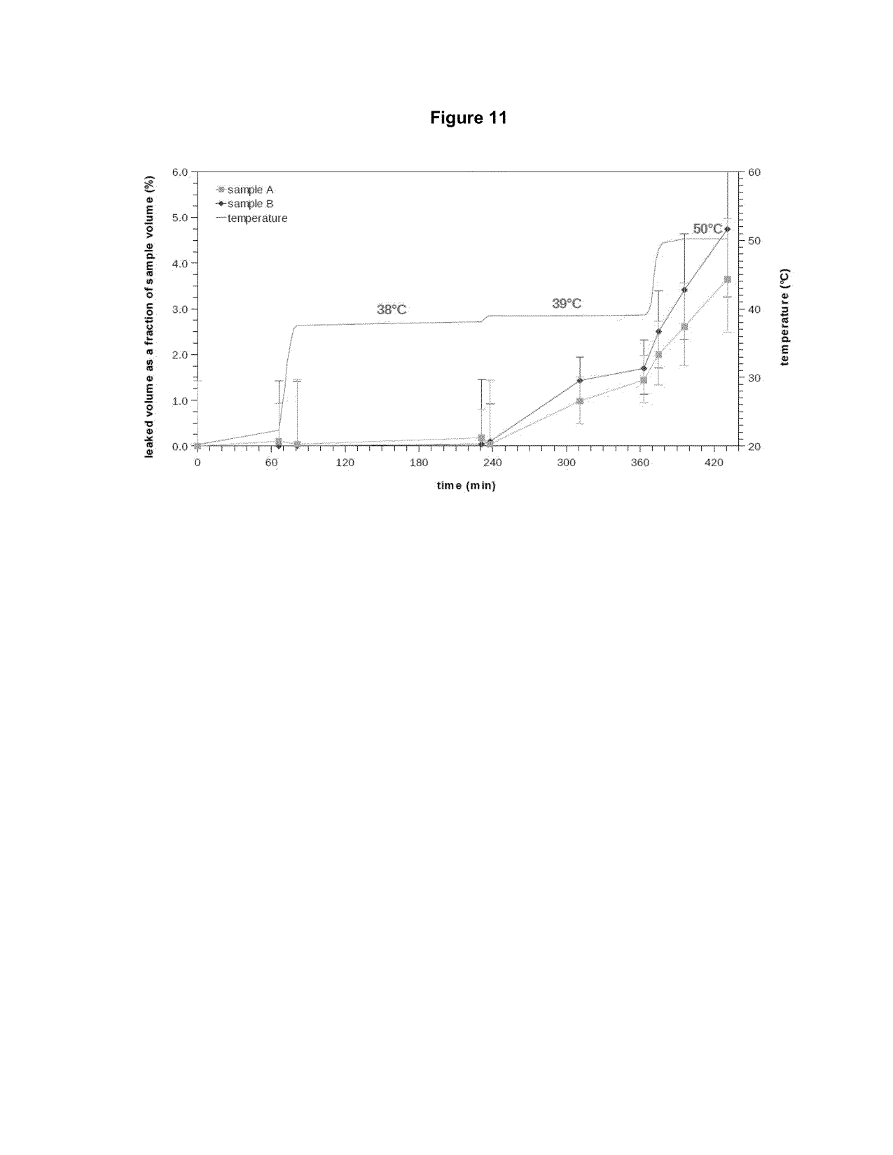

All results are summarized in an overview bar-chart in FIG. 10.

### Samples Stored at 6° C.

The fridge-samples were stored a total of 200 days for DPPC liposomes and 121 days for DPPC/DMPC liposomes. For both liposome types, chlorite/chlorate was not detected in the storage medium.

### Room-Temperature Samples

Room temperature samples were stored for a total number of 158 days for DPPC liposomes and 79 days for DPPC/DMPC liposomes. In all cases chlorite/chlorate was detected in the storage medium and increased continuously over time.

### DPPC Liposomes

The results remained below the LOD for at least 101, 28 and 79 days for SX123, SX124 and SX125, respectively. The period between the second and the third measurement (28 and 79 days) was relatively long, such that SX124 exceeded LOD as well as LOQ in this period. SX123 never exceeded LOQ at all, while SX125 did so between the 77^{th} and 101^{st} day of storage.

### DPPC/DMPC Liposomes

The results for pure WF10 encapsulating Liposomes (SX130) remained below the LOD for at least 51 days. Readings never exceeded the LOQ. For SX132 (PBS) and SX131 (taurine), LOD was exceeded between days 22 and 51, followed by an overrun of the LOQ between day 51 and 79.

### Discussion & Conclusion

With these LTS-experiments, it was possible to get a view of the long-term behavior of WF10 liposomes and to show that DPPC liposomes could be stored at 6° C. without significant leakage for at least 200 days. The same was true for DPPC/DMPC liposomes within a period of 121 days. At room temperature, the sample exhibited leakage after 80 days.

### Example 12

### Characterization of the Leakage Behavior of DPPC Liposomes

The lipid phase transition temperature (LPTT) is a consideration when it comes to long-term storage of liposomal encapsulated material. Knowledge about the leakage processes around this temperature is useful when designing formulations to encapsulate chlorite and/or chlorate. In this example, the results of leakage studies on pure DPPC liposomes are reported.

### Preparation and Experiments

### Samples

Two similar samples were prepared from 1,2-dipalmitoyl-sn-glycero-3-phosphocholine, following the preparation procedure described in Example 6. The samples are SX122 and SX126 and have a lipid concentration of about 40 mM. Detailed parameters are given below:

### SX122

12.0 mL of a 25 mg/mL DPPC-chloroform-solution was added to a round-bottom flask. This corresponds to a lipid mass of 300 mg. DPPC was purchased from Avanti Polar Lipids (Product no.: 850355C). Hydration was done with 10.22 mL WF10 prepared from OXO-K993 25.03 g OXO-K993 in 250 mL solution. During the hydration process, the sample was heated to 42° C. for 5 min. Extrusion took place at 45.4° C. and lasted 20 min. 10 consecutive passes were performed and the sample was stored overnight at 6° C. in a fridge. Dialysis steps were performed in 900 mL saline solution, prepared with 45.004 g NaCl per 5000 mL solution. 5 subsequent steps lasting 74, 73, 38, 39 and 69 min were carried out. Afterwards, the sample was stored at 6° C. for later use.

### SX126

14 mL of a 25 mg/mL DPPC-chloroform-solution was added to a round-bottom flask. This corresponds to a lipid mass of 350 mg. DPPC was purchased from Avanti Polar Lipids (Product no.: 850355C). Hydration was done with 11.9 mL WF10 prepared from OXO-K993 25.03 g OXO-K993 in 250 mL solution. OXO-K993 was received from Nuvo Research GmbH. During the hydration process, the sample was heated to 46±+3° C. for 4 min. Extrusion took place at 46±1° C. and lasted 12 minutes. 10 consecutive passes were performed. Afterward samples were stored over the weekend at 6° C. in a fridge. Dialysis steps were performed in 900 mL saline solution (0.9%), prepared from 45.08 g NaCl per 5000 mL solution. 5 subsequent steps lasting 78, 68, 68, 85 and 51 min were carried out. Afterwards, the samples were stored at 6° C. for later use.

### HRL Experiments

HRL experiments where designed to monitor liposome leakage over a range of few hours to a maximum of a few days. It was possible to see changes of leakage behavior with a time resolution >5 min. Additionally, the temperature of interest could be precisely adjusted between room temperature and 60° C. in steps of 0.1 K.

The HRL setup included the exact control of the temperature as well as the possibility to stir the storage medium. As a storage vessel, a 500 mL measuring cylinder was used, filled with 200 mL 0.9% saline solution as storage medium. The sample was introduced to a Slide-A-Lyzer™ dialysis frame, as was done in the LTS setup (Example 11), which was then immersed into the storage solution. The frame was held up with a foam oat buoy as was also done during dialysis. A magnetic stirrer constantly stirred the storage solution. The measuring cylinders were placed in a Julabo U3/B heat bath which controlled the temperature with a precision of about 0.1K, for periods <12 h and 0.3K for periods >12 h. Temperature was monitored with officially calibrated thermometers having a precision of about 0.1K. The setup enabled the monitoring of two samples in separate storage cylinders. Usually two identical samples were observed to provide reliable data.

The following HRL experiments were performed:

| Date | Sample | Type |
|---|---|---|
| Dec. 12, 2011 | SX122 | temp. scan |
| Dec. 13, 2011 | SX122 | HRL at 41° C. |
| Jan. 6, 2012 | SX122 | HRL at 38° C. |
| Jan. 11, 2012 | SX122 | HRL at 39° C. |
| Jan. 25, 2012 | SX126 | HRL at 41° C. |
| Jan. 26, 2012 | SX126 | HRL at 39° C. |
| Feb. 22, 2012 | SX126 | HRL at 40° C. |

### Results and Discussion

The results of the experiments are depicted in FIGS. 11-15. Here the leaked amount of WF10 is plotted versus the time, elapsed since the beginning of the experiment. The temperature is also displayed on the right hand y-axis. The leaked WF10-amount is given as a fraction of the total sample volume, i.e. a value of 2% for a 1 mL sample, for example, corresponds to a total release of 20 μL of WF10 from the sample's liposomes.

As previously mentioned, every experiment was usually performed with two separate identical samples at a time. The samples are called ‘sample A’ and ‘sample B’ in the graphs.

### Leakage Starts at 39° C.

On a scale of a few hours, significant leakage starts at temperatures above 38° C. FIG. 11 shows the results of a temperature scan. Temperature was held at 38° C. for about three hours with no measurable change in the concentration of WF10 in the outer medium. Then, the temperature was raised by 1K inducing a leakage of WF10 from the liposomes within only a few minutes. Further heating to ˜50° C. increased the rate of leakage.

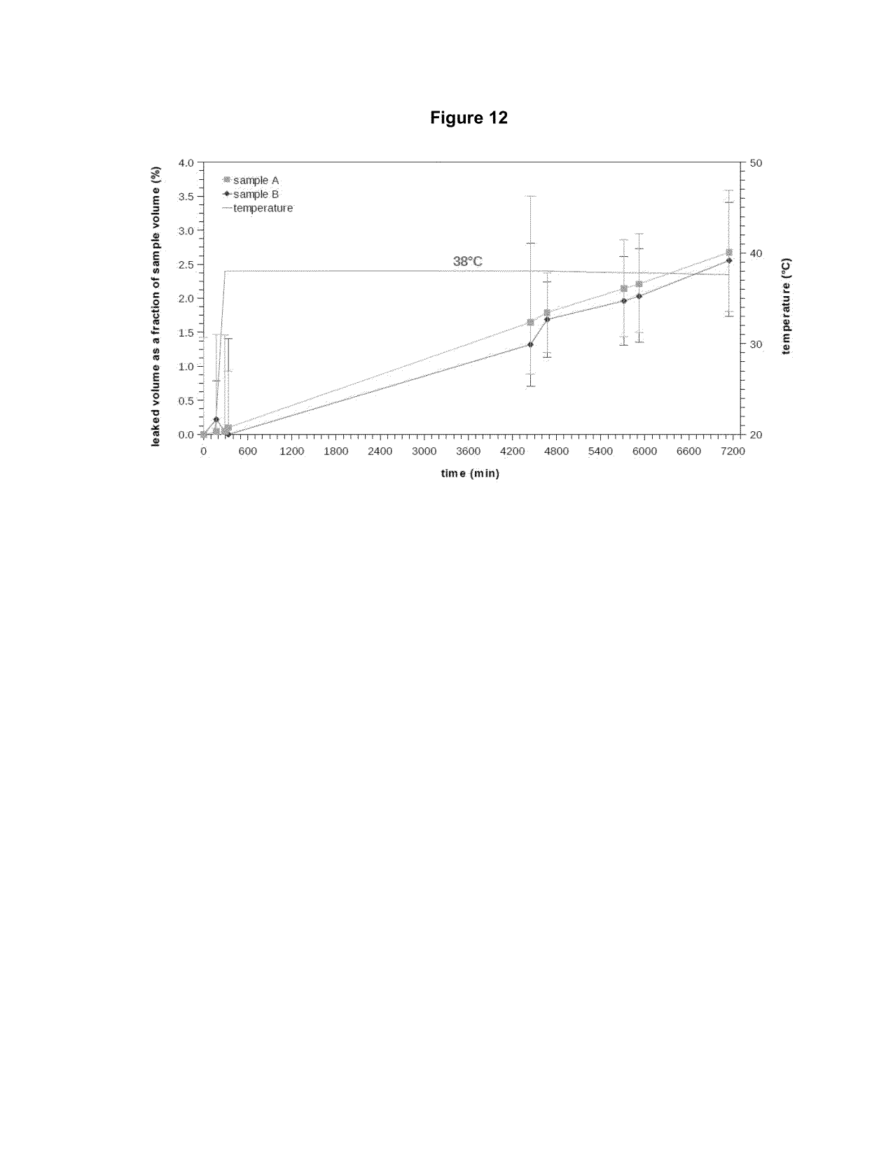

However, FIG. 12 shows the result of an HRL experiment at 38° C. over 5 days, indicating a slight leakage to approximately 2.5% of the total sample volume. The maximum capacity of this sample (SX122) was in the range of 8-9%, such that only one third of the enclosed amount was released at this time.

### Partial Leakage at 39° C.

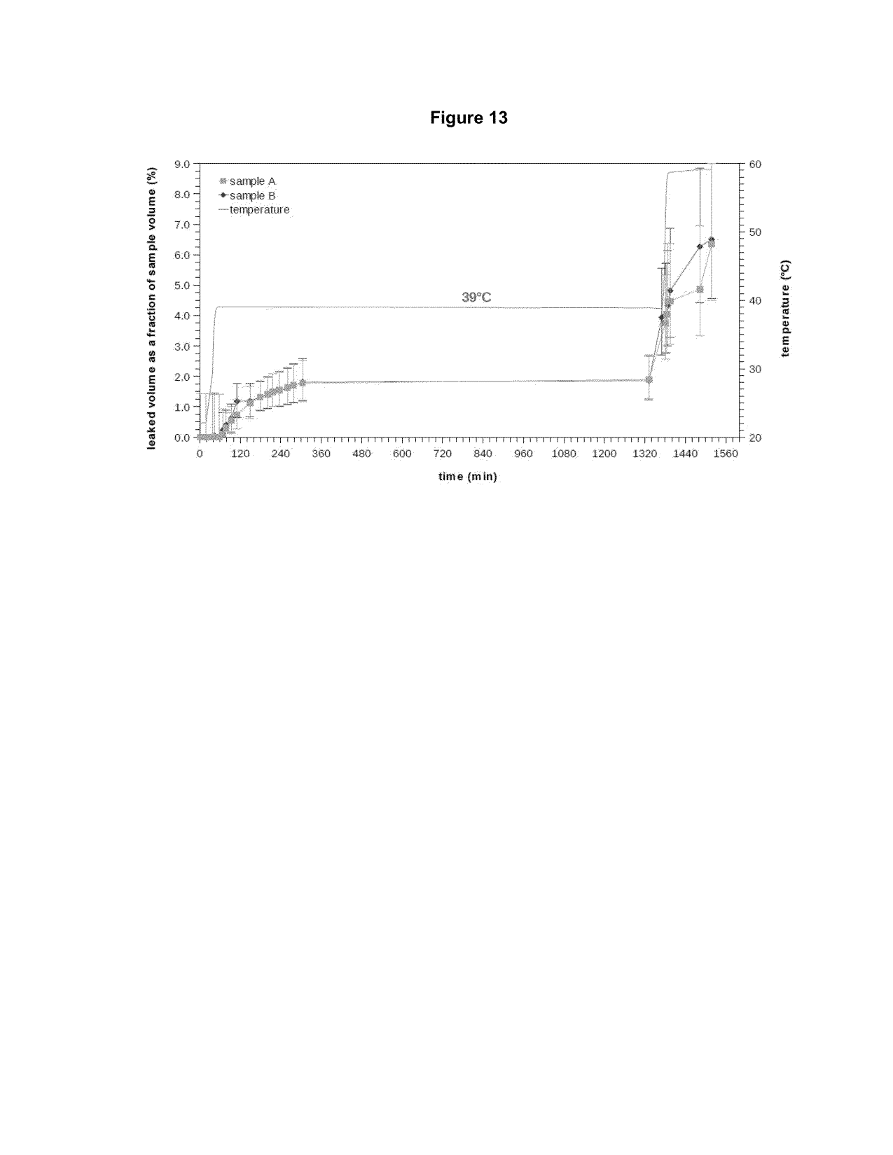

FIG. 13 shows a HRL experiment at 39° C. It can be seen that WF10 with a volume of about 2% of the total sample volume was released after ca. 4 hours. Further, leakage did not appear, even after leaving the experimental setup at 39° C. overnight. Only after further heating, did the rest of the enclosed WF10 leak, until a volume of about 6-7% of the sample volume was released.

### Leakage Above 39° C.

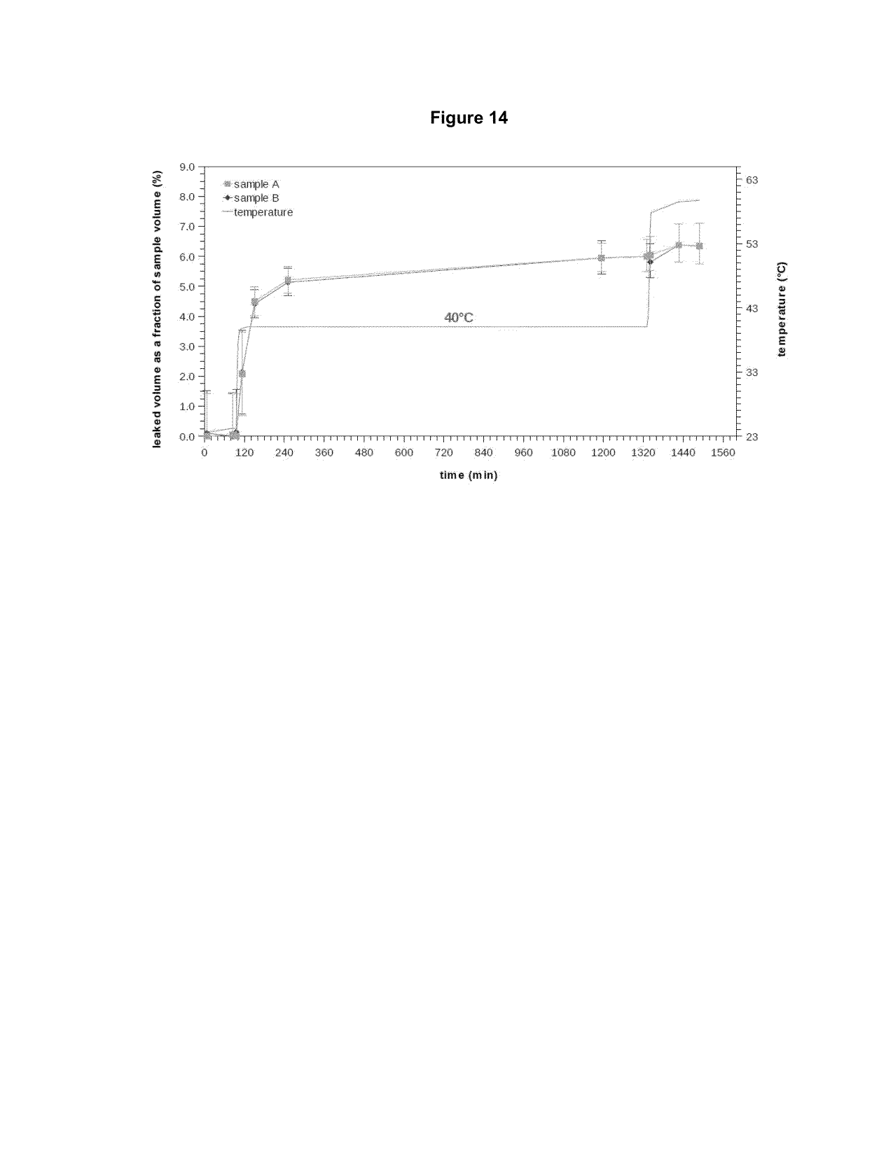

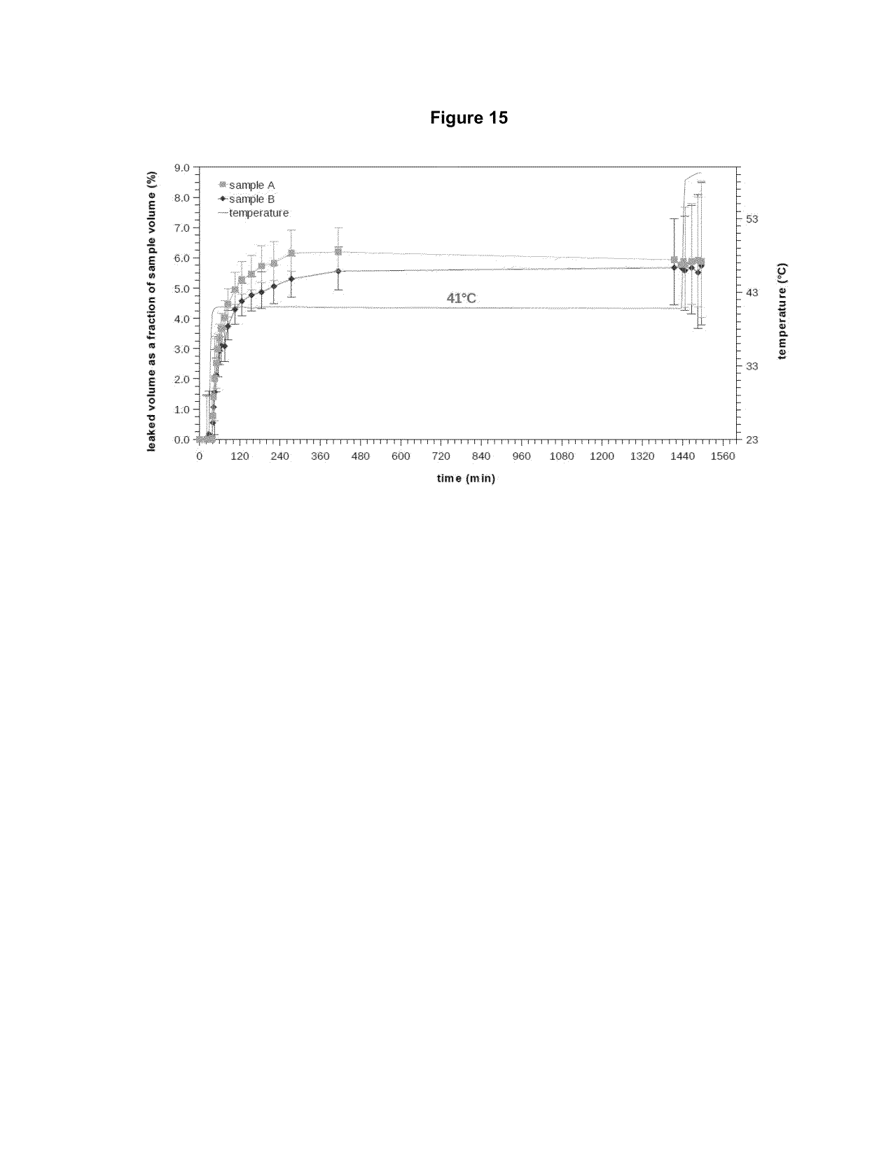

FIGS. 14 and 15 illustrate the results of the HRL experiments at 40 and 41° C., respectively. The full amount of enclosed substance was released over a period of about 4 hours.

### Enclosed Volume

The volume enclosed by the liposomes in SX122 was found to be approximately 8-9% of the sample volume. For SX126, it was 6-7% and, thus, lower. Even though the samples went through the same preparation procedure with nearly identical parameters, the difference was not negligible.

### Stability

For both samples, the duration between preparation and last experiment was approximately one month during which no leakage occurred in the storage vessel at 6° C. Therefore, it can be concluded that DPPC liposomes are stable for at least one month under constant cooling.

### Example 13

### 4-Week Stability Study

### (a) Manufacture and Characterization of the Solutions

Materials

| | |
|---|---|
| Sodium chlorite (solid) | Clariant, |
| Sodium chlorite (liquid, 25%) | Merck |
| Sodium chloride | Merck |
| Sodium chlorate | Sigma-Aldrich, A.C.S., |
| Sodium sulfate anhydrous | Sigma-Aldrich, A.C.S., |
| Sodium carbonate anhydrous | Merck |
| 1N Sodium hydroxide | Merck, TitriPur ™, |
| Di-sodium hydrogenphosphate anhyd. | Merck |
| Sodium dihydrogenphosphate | Merck |
| monohydrate | |
| Water for Injection (WFI) | Fresenius |

### Osmotic Stress Considerations

Due to their non-ideal behavior, the “real” (experimental) osmotic pressure of salt solutions cannot be calculated from their molal concentrations with the necessary precision. Therefore, the experimental value for a solution's osmotic pressure (osmolality) is expected to deviate from the theoretical value (osmolarity) by approximately −5 to −10%. The typical theoretical value reported for the osmolarity of saline (0.9% sodium chloride) is 308 mosmol/L. This should correspond to an osmolality of 310.8 mosmol/kg. It is commonly assumed this would be the “real” value.

The specified osmolality for WF10 is 290 to 330 mosmol/kg (mean: 310) as the experimental value. This apparently matches the value of saline, be it 308 mosmol/L (osmolarity) or 310.8 mosmol/kg (osmolality). However, it has been established experimentally that, for saline, the real value is 287 mosmol/kg (mean from: 290; 287; 284).

In order to avoid osmotic stress, the osmolalities of the solutions to be manufactured were designed to be close to the value of an ideal WF10, which was 310 mosmol/kg, in order to minimize the difference from the value for saline, which is 287 mosmol/kg. This was possible only by extensive experimental work.

Sample 1: Isotonic WF10 with no Chlorite (Note TCDO Referred to in this Sample was OXO-K993)

Starting Concentrate: TCDO with No Chlorite (yTCDO)

| Salt | Weight [g] |
|---|---|
| Sodium chlorite (solid) | 0 |
| Sodium chloride | 1.649 |
| Sodium chlorate | 0.957 |
| Sodium sulfate* | 0.518 |
| Sodium carbonate* | 0.353 |
| 1N Sodium hydroxide | 6.0 mL |
| Water^{#} to make a total of | 50.0 |

*anhydrous;
^{#}Water for injection

7.84 g of yTCDO was diluted in water for injection to make up a final volume of 50.0 mL.

Sample 2: Isotonic WF10 with No Chlorate (Note TCDO Referred to in this Sample was OXO-K993)

Starting Concentrate: TCDO with No Chlorate (zTCDO)

| Salt | Weight [g] |
|---|---|
| Sodium chlorite (solid) | 3.540 |
| Sodium chloride | 1.295 |
| Sodium chlorate | 0 |
| Sodium sulfate* | 0.394 |
| Sodium carbonate* | 0.141 |
| 1N Sodium hydroxide | 6.0 mL |
| WFI^{#} to make a total of | 50.0 |

*anhydrous;
^{#}Water for injection

5.44 g of zTCDO was diluted in water for injection to make up a final volume of 50.0 mL.

### Sample 3: Isotonic Unbuffered Chlorate ClO₃⁻

883 mg of sodium chlorate (equivalent to 662.2 mg of chlorate) was dissolved in 50 g of water for injection.

### Sample 4: Isotonic Chlorite Buffered to pH 7.4

(i) Phosphate Buffer pH 7.2

Solution 1: 2.84 g of di-sodium hydrogenphosphate (Na₂HPO₄, anhydrous) was dissolved in 100 g of water for injection.

Solution 2: 2.76 g of sodium dihydrogenphosphate (NaH₂PO₄.H₂O) was dissolved in 100 g of water for injection.

Buffer pH 7.2: Under control of a glass electrode, Solution 2 was added to Solution 1 until a pH of 7.21 was reached.

Osmolality: 403 mosmol/kg (mean from 400; 406).

(ii) Sodium Chlorite Solution

9.0 g of commercially available sodium chlorite solution 25% (wt/wt) was diluted with 93 g of water for injection.

Osmolality: 506.5 mosmol/kg (mean from 504; 509)

(iii) Isotonic Phosphate Buffer pH 7.2

38.0 g of phosphate buffer pH 7.2 from (a) was made up to 50.0 g with water for injection.

Osmolality: 310 mosmol/kg (mean from 309; 310; 311)

(iv) Isotonic Sodium Chlorite Solution

30.3 g of the sodium chlorite solution from (ii) was made up to 50.0 g with water for injection.

Osmolality: 294.7 mosmol/kg (mean from 293; 295; 296)

(v) Isotonic Chlorite Solution Buffered at pH 7.4

Under control of a glass electrode, the isotonic phosphate buffer pH 7.2 from (iii) was added to the isotonic sodium chlorite solution from (iv) (50 g); until a pH of 7.39 was reached.

The analytical characterization of the prepared solutions is reported in Table 3.

### (b) Preparation of Liposomes Encapsulating the Prepared Solutions

Liposome samples were prepared using one or two of the following phospholipids:
1,2-dimyristoyl-sn-glycero-3-phosphocholine DMPC, purchased as powder from Lipoid GmbH (LIPOID PC 14:0/14:0), of which 801 mg were dissolved in 60 mL chloroform (13.35 mg/mL)
   1,2-dipalmitoyl-sn-glycero-3-phosphocholine DPPC, purchased as powder from Lipoid GmbH (LIPOID PC 16:0/16:0), of which 10.617 g were dissolved in 126 mL chloroform (84.26 mg/mL)
   hydrogenated phosphatidylcholine (soy) Hydro Soy PC, purchased as powder from Lipoid GmbH (LIPOID S PC-3) of which 1,208 mg were dissolved in 25 mL chloroform (48.32 mg/mL)

Samples 1-4 were encapsulated in the above mentioned lipids. In order to avoid osmotic stress (inside vs. outside), these solutions were made to be isotonic (approx. 300 mosmol/kg). Additionally, for one of the samples, an i.v. solution of WF10 (Immunokine™) manufactured by Pharma Hameln, Germany was used. Also prepared was a 0.9% saline solution from sodium chloride and deionized water (NaCl from VWR, GPR Rectapure)

13 liposomal samples were prepared for the experiments, and were identified as follows:

| Name | content | lipid |
|---|---|---|
| Sample 5 | Sample 2 | DPPC |
| Sample 6 | Sample 3 | DPPC |
| Sample 7 | Sample 1 | DPPC |
| Sample 8 | Sample 4 | DPPC |
| Sample 9 | Sample 2 | DPPC/DMPC (9 mol/1 mol) |
| Sample 10 | Sample 3 | DPPC/DMPC (9 mol/1 mol) |
| Sample 11 | Sample 1 | DPPC/DMPC (9 mol/1 mol) |
| Sample 12 | Sample 4 | DPPC/DMPC (9 mol/1 mol) |
| Sample 13 | Sample 2 | Hydro Soy PC (S PC-3) |
| Sample 14 | Sample 3 | Hydro Soy PC (S PC-3) |
| Sample 15 | Sample 1 | Hydro Soy PC (S PC-3) |
| Sample 16 | Sample 4 | Hydro Soy PC (S PC-3) |
| Sample 17 | WF10 | Hydro Soy PC (S PC-3) |

### Sample Preparation

Samples were prepared using the procedures generally described in Example 6 with some modifications. The precise parameters are provided in the subsections below.

### Preparation Timeline

The following provides a chronology of the whole sample preparation:

Sep. 5, 2012 Evaporation, hydration & extrusion for Samples 5, 6, 7, 9, 10 and 11
Sep. 6, 2012 Evaporation, hydration & extrusion for Samples 8, 12, 13, 14, 15, 16 and 17

### Sep. 10, 2012 Dialysis of Samples 5-8

### Sep. 11, 2012 Dialysis of Samples 9-12

### Sep. 12, 2012 Dialysis of Samples 13-17

### Sep. 14, 2012 Dialysis of Sample 17

All samples or partly finished samples were stored in a fridge (1-4° C.) at the end of the preparation or between the different preparation steps, respectively.

### Evaporation & Hydration

For DPPC samples, 2000 μL of the DPPC-chloroform-solution (84.26 mg/mL) was evaporated and hydrated with 574 μL of the corresponding ion-mixture.

DPPC/DMPC samples were prepared by evaporating 1300 μL of the DPPC-chloroform-solution and 2000 μL of the DMPC-chloroform-solution (13.35 mg/mL). 6390 μL of the corresponding ion-mixture was used for hydration.

In both cases hydration took place at 47° C. and lasted less than 5 minutes.

For hydro soy PC samples, the hydration temperature was set to 60° C., while the duration of the procedure was less than 5 minutes, as well. 4000 μL of the lipid-chloroform-solution (48.32 mg/mL) was evaporated prior to hydration, which took place with 6170 μL of the corresponding ion-mixture.

### Extrusion

Extrusion took place at a temperature of 47° C. for DPPC and DPPC/DMPC samples and at 60° C. for hydro soy PC samples. 10 successive extrusion steps were applied to all samples but Sample 17, where 9 successive steps were applied. The overall duration for those steps was less than 15 minutes for all Samples but 13, 14, and 16, where it was less than 25 minutes.

### Dialysis

Dialysis medium was 0.9% saline solution, prepared by adding 45.0 g NaCl to a 5000 mL volumetric flask, which was then completed to volume with deionized water.

All samples were dialyzed in a 3-12 mL dialysis frame. 5 successive steps were performed, each with (900 mL of dialysis medium and a duration of at least 45 min. Note that a new method of dialysis is available that utilizes microdialysis (Roth Mini-Dialyzer). It requires sample volumes (i.e., liposome preparation) in the range of only 100 μL and a dialysis volume (i.e., saline) in the range of 1-2 mL. Using this method provides an enhancement in the limit of quantitation by a factor of approximately 10 compared to the previous method.

### 4-Week Storage

The LTS-setup as described in Example 11 has been replaced by the following general method: After dialysis, the samples were removed from the dialysis frame and were stored in whatever vial seems appropriate. The separation of the liposomes from their outer medium took place on the day of examination, where a fraction of the sample was withdrawn and subjected to microdialysis, followed by monitoring the potentially leaked components in the dialysate. Details are presented in the following.

On Sep. 17, 2012, 4 mL of each sample was equally distributed over four 1.5 mL vessels (1 mL each). Two of those vessels where then stored in a fridge at 1-4° C. while the other two remained at room temperature. All vessels stored at the same temperature were appropriately labeled and placed in the same container together with a temperature logger, which was programmed to record the temperature every 5 minutes. Afterwards, the containers were sealed and stored at their respective temperatures. The log data recorded constant temperatures between 1-4° C. for the fridge samples and 21-23° C. for the samples stored at room temperature.

To ensure the integrity of all samples at the beginning of the experiment, 100 μL of the sample volumes was removed and the outer medium separated as described in the next section. These samples were then stored at −25° C. to perform an o-tolidin-analysis along with the other samples after the experiment.

The containers were reopened on Oct. 24, 2012 and 100 mL of sample was removed from each vessel to perform an o-tolidin-analysis of the outer sample medium. The procedure is described in the following subsection.

### Outer Medium Separation and Analysis

As mentioned before and contrary to the LTS-setup described in Examples 8 and 11, the samples were not stored in the dialysis chamber during an LTS experiment, but in appropriate vessels. Therefore, separation of the liposomes from their outer medium was needed before leaked substances were detected in the outer medium. For this separation small dialysis units with a maximum volume of 100 μL were used (ZelluTrans/Roth Mini-Dyalyzer, MWCO 12000 by Carl Roth, Prod. no.: 4775.1). The dialysis was performed against 1500 μL of 0.9% saline solution as dialysis medium and took place in closed 2 mL reaction vessels. For the current experiments, 100 μL of each sample was filled into the dialysis units on Oct. 24, 2012 and were left at the corresponding temperatures (either room or 1-4° C.) until Oct. 29, 2012. On this day, the dialysis units were transferred to another 2 mL reaction vessel, filled with 1500 μL of 0.9% saline solution. The “old” 2 mL vessels where then stored overnight at 1-4° C. The “new” setup was heated to 60° C. and left overnight, to force a complete leakage.

The initial samples, taken on Sep. 17, 2012, were subjected to an overnight separation procedure on this day. On the next day, the dialysis units were removed and the vials with the separated outer medium were stored at −25° C., to be analyzed along with the other samples.

On Oct. 30, 2012, an o-tolidin-analysis of the dialysis medium was performed for all three cases (initial integrity test, medium after storage and medium after storage and heating) following the descriptions of the o-tolidin-procedure in Example 7, Method B.

The concentrations of the components were not calculated. Instead, the procedure was restricted to monitoring absorption values in order to make the decision: leakage or not. This is due to the fact, that all of the quantitation methods refer to pure WF10 which has a chlorite/chlorate content different from most of the encapsulated substances. Comparing the absorption data from measurements before and after the forced complete leakage can give a rough estimation of the extent of the leakage.

### Results and Analysis

The absorbance values were comparable among each other but were not normalized with respect to a standard optical path length of 1 cm. For the absorption measurements the limit of detection (LOD), defined in Example 14 (0.095), was used. In cases where the absorbance values were below the LOD, leakage was too weak to be detected or did not take place at all. Table 4 provides the numeric values of the absorption measurements.

The values for each sample were the average of the two separately stored vessels. The results from the forced leakage are included, showing a huge signal compared with all other signals, indicating, that all samples contained properly filled liposomes. The signal from all initial measurements was below the LOD and, therefore, illustrates, that no sample contained already leaked substance in the outer medium. Concerning the individual results, three perspectives should be considered: Storage temperature, lipid composition and enclosed substance.

### Results by Storage Temperature

Storage temperature had the strongest impact on the samples' leakage behavior. All leakage, except in one case, occurred in samples stored at room temperature, and in the case of the only leaking fridge-sample (Sample 8), its leakage at room temperature exhibited the strongest signal of all samples in the test.

### Results by Enclosed Substance

All liposomes which exhibited signals above the LOD were either filled with chlorite or chlorate. Samples, filled with chlorate leaked in all room temperature scenarios and even at fridge-temperature one sample (Sample 8) slightly exceeded the LOD. For chlorite all of the room temperature samples but one (Sample 14) did so.

### Results by Lipid

Leakage was much weaker in samples made with hydro soy PC(S PC-3) which has a higher PTT (55° C.) than DPPC (41° C.) and DMPC (23° C.). In case of DPPC/DMPC mixture, the PTT is not that clearly defined and spans a temperature range between the PTT's of DPPC and DMPC.

### Example 14

### Osmolarity Effects on WHO Encapsulating Liposomes

It is known that lipid membranes are, to a certain extent, permeable to small molecules, such as water, whereas large molecules as well as charged ones cannot pass such a membrane, or do it at much lower rates. Osmosis is a direct consequence of this effect. It appears at a semipermeable boundary between two aqueous reservoirs with different concentrations of osmotic particles (e.g. larger molecules). The systems tend to equilibrate both concentrations, which is only possible by diluting the higher concentrated reservoir with water molecules from the lower concentrated one. Moving dissolved particles to the lower concentrated reservoir to raise the concentration there is not possible, since the particles are not able to pass the boundary. The consequence of the “invading” water molecules is an increase in volume of the higher concentrated reservoir, which leads to a pressure on the reservoir walls. Eventually this pressure might become too high for the wall to withstand and it will burst.

The bilayer hull of a liposome is a semipermeable membrane and therefore prone to osmotic effects. One might suggest that osmotic gradients may induce leakage of the liposomes or even cause them to burst. In a brief experiment, the leaking behavior of WF10 filled liposomes was studied, where concentrated WF10 was used to increase the osmolarity of the interior with respect to the surrounding saline medium.

The aim of this study was not to gain detailed information about leakage parameters under osmotic effects, but to find a general tendency of leakage under such conditions.

### Samples

Two samples were prepared, using 2× or 3× concentrated WF10 prepared with OXO-K993 solution provided by Nuvo Research GmbH. (Lot 4053) in the following way:

2×WF10: 10 g OXO-K993 completed to 50 mL with water
3×WF10: 15 g OXO-K993 completed to 50 mL with water

Sample preparation started on Jul. 4, 2012 and was performed according to the usual procedure (Example 6). 2.1 mL of a 84.26 mg/mL DPPC-chloroform-solution was added to the round-bottom-flask. This corresponds to a lipid mass of 177 mg. DPPC was purchased from LIPOID GmbH. Hydration was done with 6.0 mL of the respective WF10 concentration (2× or 3×) prepared as described above. During the hydration process, the sample was heated to 46±3 C for approximately 5 min. Extrusion took place at 50±3° C. and lasted approximately 20 min, including all 10 consecutive passes. Afterward samples were stored overnight at 6° C. in a fridge. Dialysis steps were performed in 900 mL saline solution, prepared with 90.034 g NaCl (for 2×WF10) and 135.063 g NaCl (for 3×WF10) per 5000 mL solution. 5 subsequent steps lasting 95, 80, 80, 60 and 80 min were carried out. Afterwards, samples were removed from the dialysis frames and where stored at 6° C.

### HRL Leakage

The HRL leakage test was started on Jul. 6, 2012 with a 50 minute dialysis step in 0.9% saline solution with 45.016 g NaCl per 5000 mL solution to remove the higher concentrated storage medium of the liposomes. Afterwards the procedure was carried out as described in Example 12 without using the heat bath, such that it took place at room temperature 22. As usual, both samples were split prior to the experiments and processed in two parallel measuring cylinders, such that for each sample, two data sets were available and plotted in the results section.

### Determination of Enclosed Volume

To determine the volume of the enclosed WF10 in the samples, full leakage was induced by heating the HRL-setup, described above, to 60° C. for several hours. An o-tolidin-analysis of the surrounding saline medium was then used to evaluate the WF10 content.

### Results & Evaluation

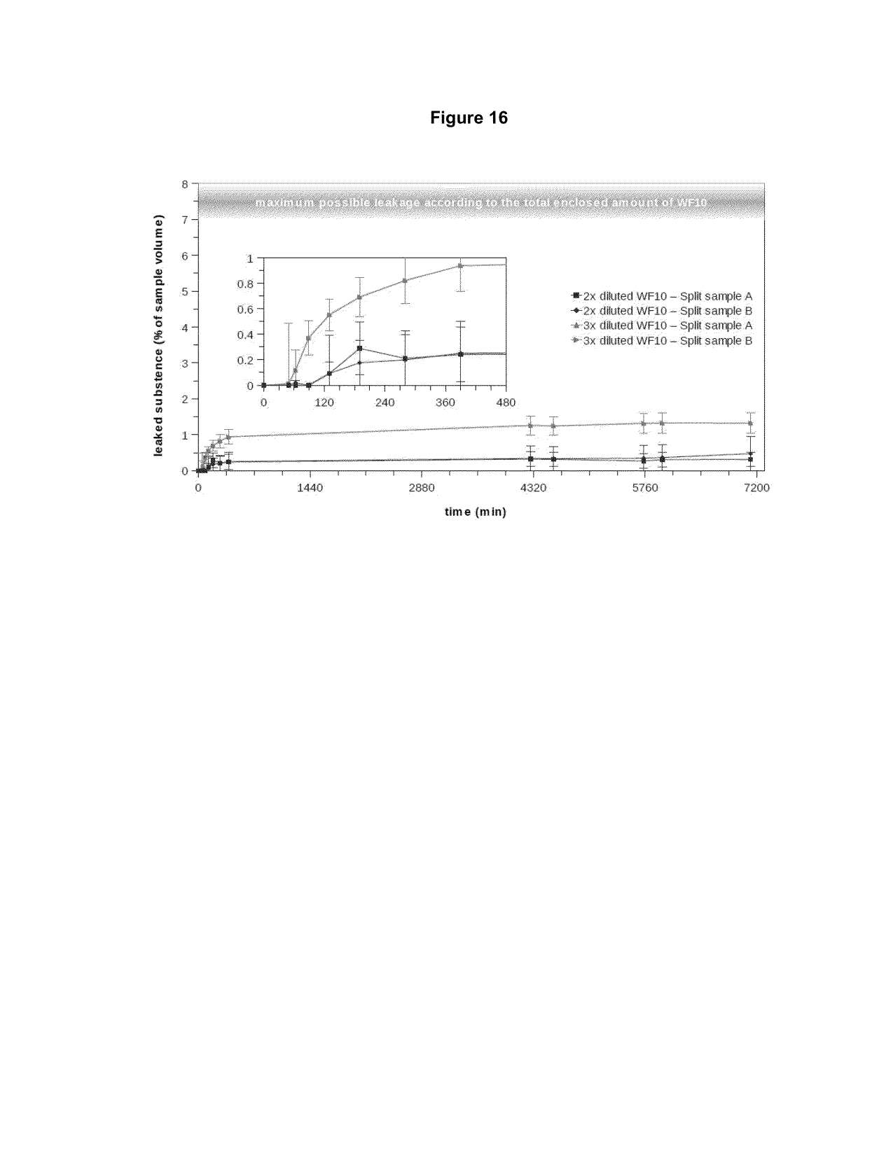

Evaluation of the medium-samples taken during the experiment was performed as described in Example 7 (Method B). The fact, that WF10 concentration was two or three times higher than usual was considered when computing the released WF10 amount from the determined WF10 concentrations in the storage medium. FIG. 16 illustrates the results in terms of leaked WF10 related to the whole sample volume. It can be seen, that leakage was detected after 1-2 hours. During the whole experimental period, approximately 5% of the enclosed double concentrated WF10 and 18% of the triple concentrated WF10 was released. Almost all leaked WF10 was released during the first 6 hours after leakage started. During the subsequent 4 days the changes where so small that no statement can be made whether leakage stopped or continued at a very slow rate.

### Example 15

### Preparation of Liposomes Using Ethanol Injection Method

WF10 containing liposomes were prepared using the crossflow ethanol injection method with the following lipid compositions: 1,2-dipalmitoyl-sn-glycero-3-phosphocholine (DPPC), DPPC with a 10% fraction of 1,2-dimyristoyl-sn-glycero-3-phosphocholine (DMPC), DPPC with a 10% fraction of 1,2-dipalmitoyl-sn-glycero-3-phosphoglycerol (DPPG) and DPPC with a 10% fraction of 1,2-Dimyristoyl-sn-glycero-3-phosphoglycerol (DMPG). The charged lipids (DPPG and DMPG) were added to prevent aggregation of liposomes during processing and storage.

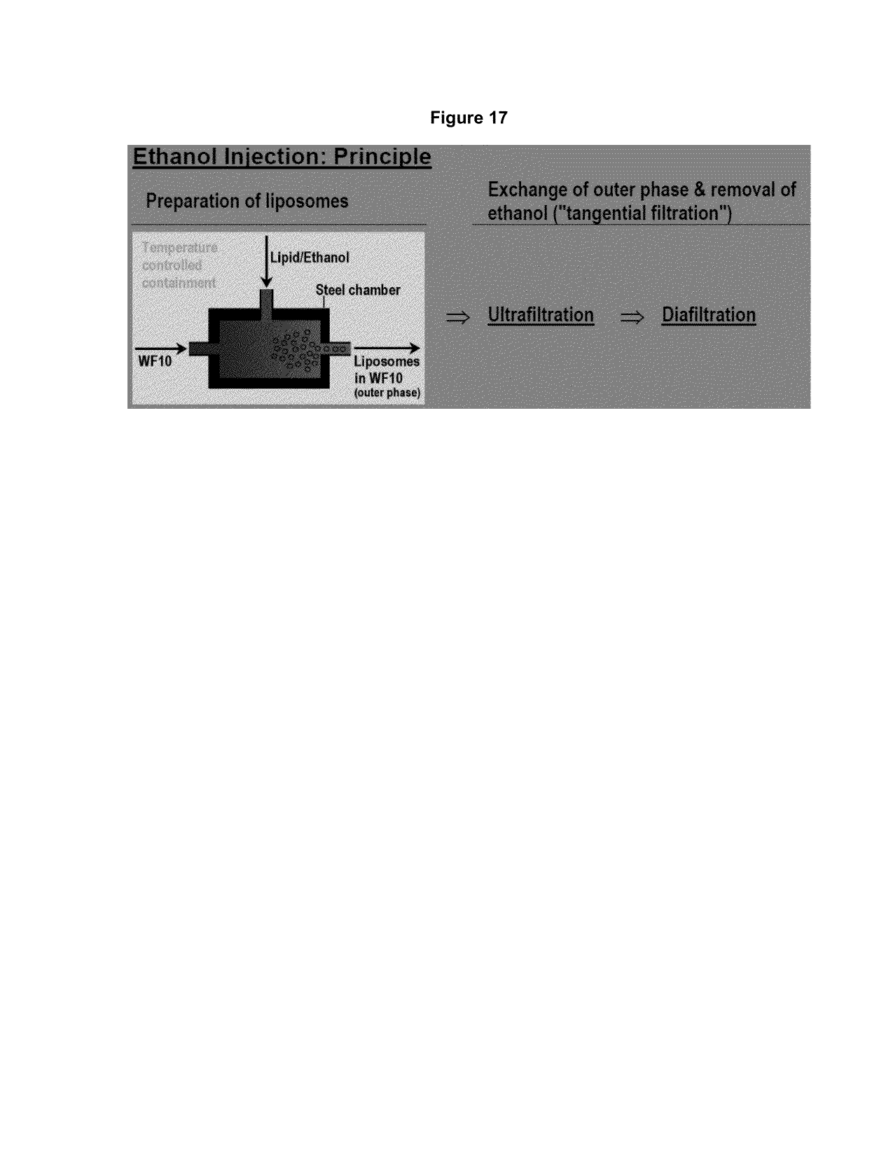

FIG. 17 shows a schematic of the preparation of liposomes using the ethanol injection method. This method results in direct formation of small unilamellar liposomes of desired size with an acceptable size distribution.

Characteristics of the applied crossflow ethanol injection method include the following
Batch size is determined (not limited) by the volume of the sample vessels
Resulting liposomes are instantly of controlled size and size distribution
Minimal thermal stress
The outer phase must be exchanged and residual ethanol must be removed:
   Ultrafiltration is applied to concentrate the primary product
   Diafiltration will
      Exchange the outer phase (WF10) by saline
      Remove the ethanol from the outer phase and from the enclosed WF10/ethanol mixture

### Materials and Methods

DPPC, DMPC, DMPG and DPPG were supplied by Lipoid GmbH (Germany). The 96% Pharm. Eur. grade ethanol for dissolving the lipid components was purchased from Merck KGaA (Germany).

The aqueous phase for the vesicle preparation was WF10 Solution—a 10% dilution of active ingredient OXO-K993 in water according to the instructions of Nuvo Manufacturing GmbH. 0.154M (physiological) NaCl Solution was used as aqueous phase for dilution. All reagents were purchased at highest purity and if possible at USP or Pharm. Eur. Grade.

Liposomes were produced with Polymun's liposome technology (crossflow injection technique) as described in Wagner et al., Journal of Liposome Research, 2002, 12(3): 259-270. Ultra-/diafiltration (small scale) was performed using a Sartocoon Slice Casette 100 kD UF membrane (Sartorius Stedim Biotech GmbH, Germany).

Measurements for the determination of liposome size were performed by Dynamic-Laser-Light-Scattering with a Malvern Nano ZS. This system was equipped with a 4 mW Helium/Neon Laser at 633 nm wavelength and measures the liposome samples with the non-invasive backscatter technology at a detection angle of 171°. Liposomes were diluted in aqueous phase to reach optimal liposome concentration and the experiments were carried out at 25° C. The results are presented in an average diameter of the liposome suspension (z-average mean) with the polydispersity index to determine liposome homogeneity. In addition, the determination of the zeta potential was also performed with the Malvern Nano ZS. Therefore, the samples were diluted with 0.154 M NaCl solution.

The quantification of DPPC was performed by reversed phase HPLC.

The quantification of WF10 was performed by o-tolidin method (see Example 7, Method B) measuring the absorbance at 442 nm.

### Experimental and Results

All experiments were performed with Polymun's liposome technology. The injection module used for all experiments was equipped with a 350 μm injection hole diameter. In the course of development, pure DPPC and DPPC/DMPC liposomes showed a small Zeta potential. Therefore two further lipid formulations (DPPC/DMPG, DPPC/DPPG) were used. Several process parameters were varied in order to study the effect on liposome properties and to optimize the process parameters to meet the requirements on the final product.

The starting conditions of the process development for the pure DPPC and the DPPC/DMPC liposomes were the following:
Temperature of the aqueous phases: 40° C.
Temperature of the lipid-ethanol solution: 40° C.
Aqueous phases for the vesicle preparation: WF10 solution
Aqueous phases for dilution: 0.154M (physiological) NaCl
Ethanol phase (lipid-ethanol solution): 75 mL for 1000 mL aqueous phase (total production volume: 1075 mL)
WF10 concentration (intermediate volume): 79.1 μg/mL

The starting conditions of the process development for the DPPC/DMPG and the DPPC/DPPG liposomes were the following:
Temperature of the aqueous phases: 55° C.
Temperature of the lipid-ethanol solution: 55° C.
Aqueous phases for the vesicle preparation: WF10 solution
Aqueous phases for dilution 0.154M (physiological) NaCl
Ethanol phase (lipid-ethanol solution): 75 mL for 1000 mL aqueous phase (total production volume: 1075 mL)
WF10 concentration (intermediate volume): 79.1 μg/mL

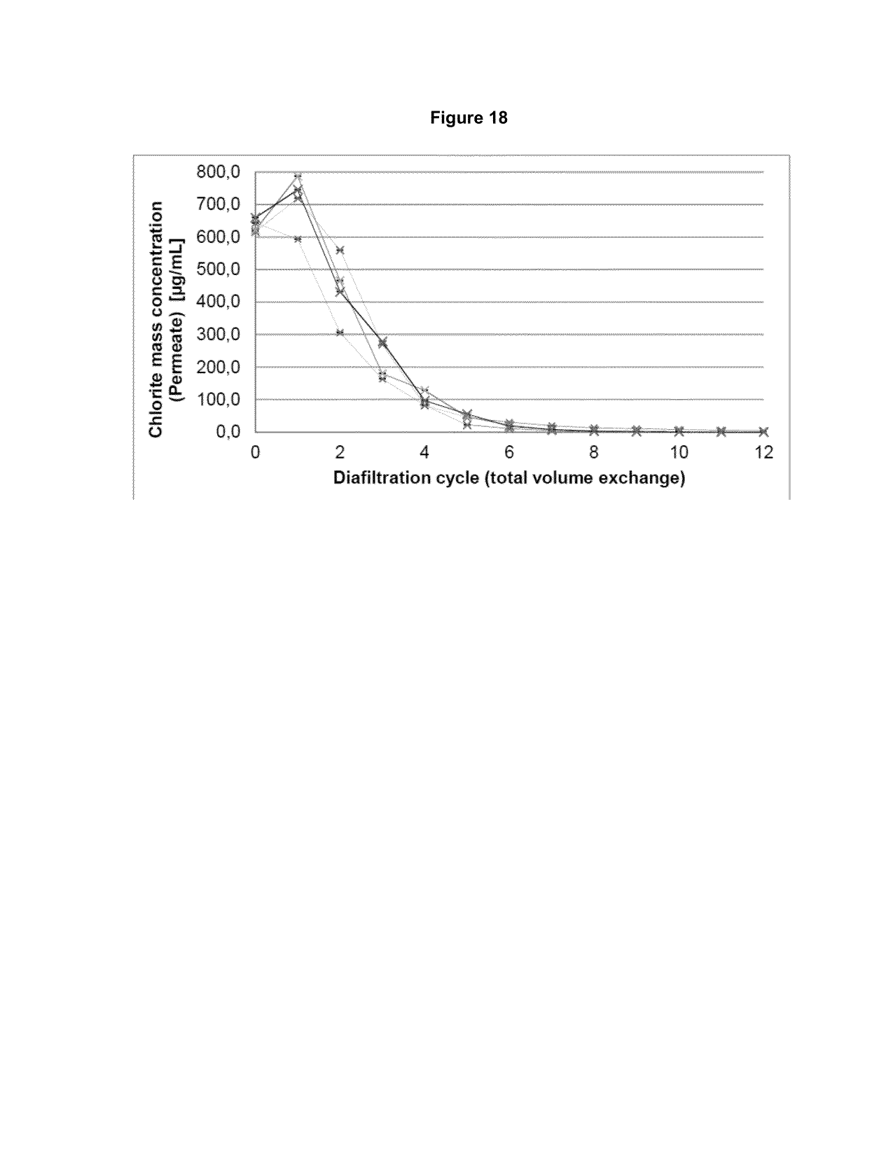

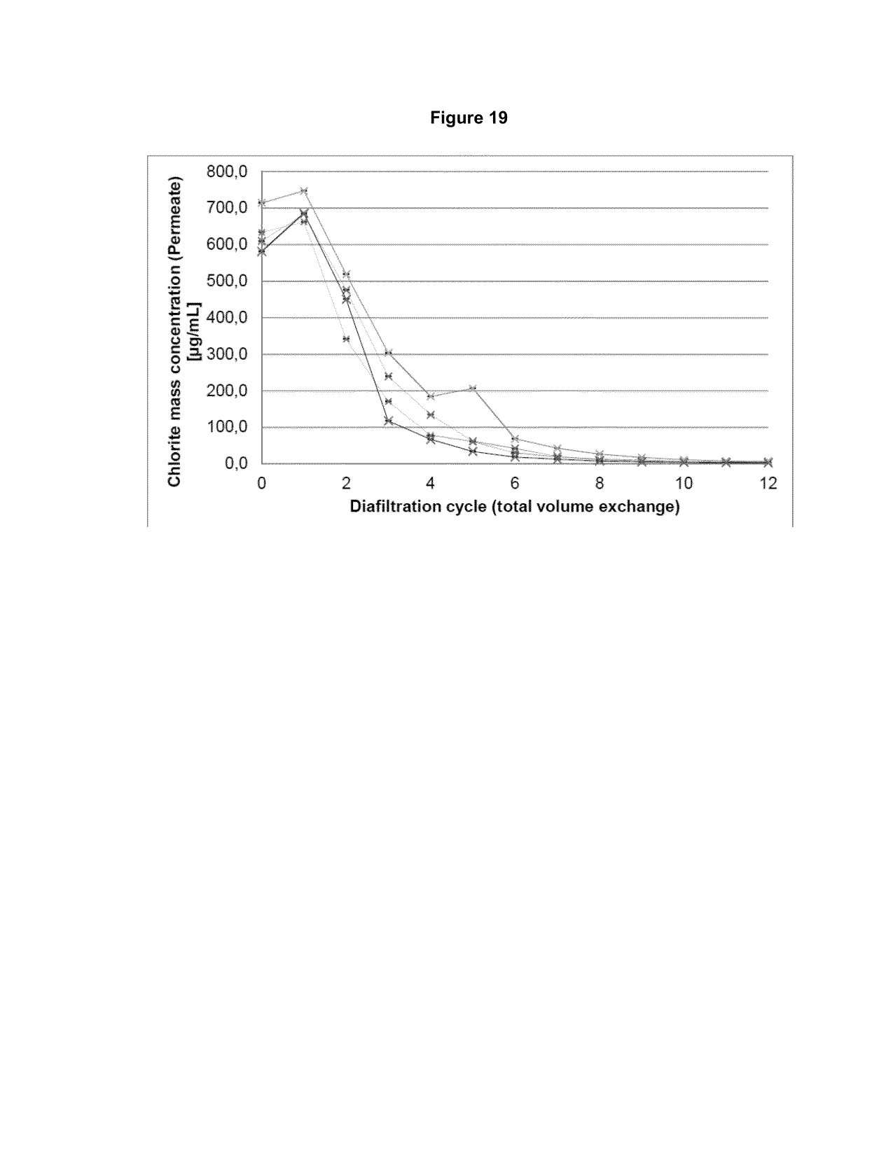

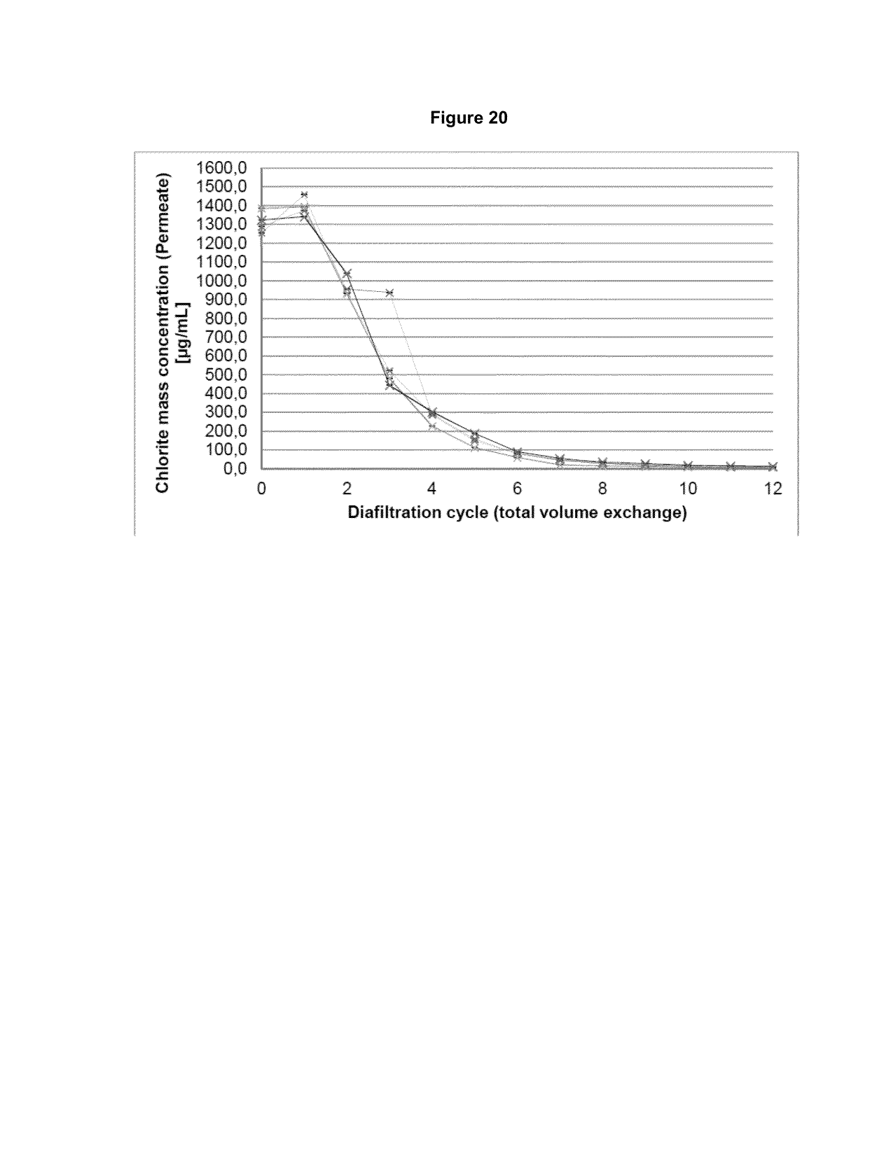

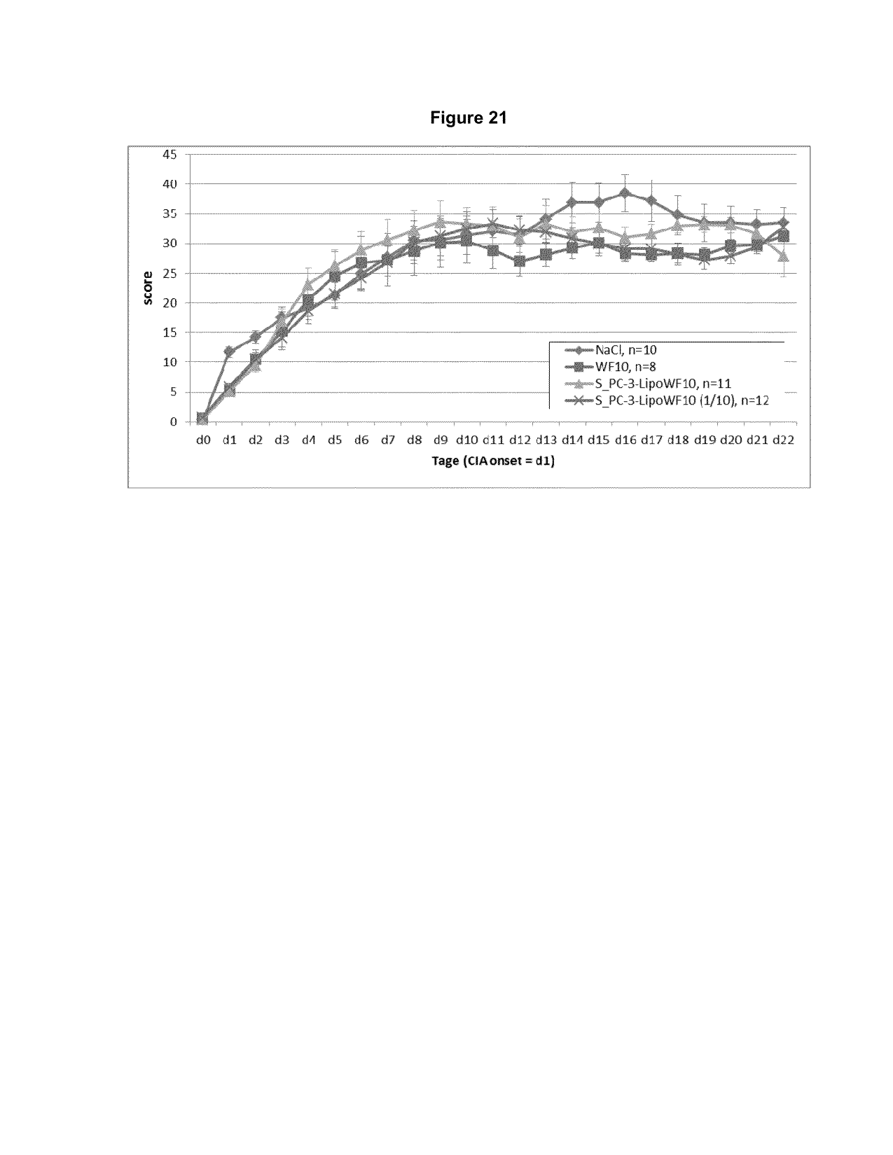

Table 5 shows the data for the various WF10-encapsulated liposomes prepared using the ethanol injection method, including liposome size, polydispersity index (PdI), DPPC quantification, and chlorite quantification. Table 6 shows the same results for a repeated run of the experiments reported in Table 5. FIGS. 18 and 19 show the amount of chlorite collected in the filtrate during the diafiltration process for the experiments reported in Tables 5 and 6, respectively. Table 7 shows the data for various WF10-encapsulated liposomes prepared using the ethanol injection method and containing 2× the concentration of WF10 compared to the liposomes prepared for Tables 5 and 6, including liposome size, polydispersity index (PdI), DPPC quantification, and chlorite quantification, and FIG. 20 shows the amount of chlorite collected in the filtrate during the diafiltration process for the same 2×WF10 liposomes.

Table 8 shows the results of a longer term stability study performed on the samples reported in Tables 6 and 7.

### Example 16

### Collagen Induced Arthritis (CIA) Animal Model Study

Female DBA/1 mice were sensitized with a collagen II solution on day 0 and boosted on day 21. The animals were scored from day 17 for clinical signs of collagen induced arthritis (CIA). If the evaluated score exceeded a value of 5, CIA was considered to be established. Animals which developed CIA within the first 35 days after the immunization were included in the study. Animals which developed CIA after this time point were excluded. From the day of the CIA onset animals were treated with WF10 (0.25 ml/kg), WF10 in liposomal form (LipoWF10), or NaCl. The scoring was done for a total of 21 days after CIA onset. After this period the animals were sacrificed.

### Study Location:

Medizinisch-Experimentelles Zentrum der Medizinischen Fakultät Leipzig, Liebigstraβe 26a, 04103 Leipzig; Max-Burger-Forschungszentrum (MBZ), Johannisallee 30, 04103 Leipzig

### Method:

Female DAB/1 mice (Janvier), 7-8 weeks old with a bodyweight of about 17 g (±2) became acclimatized in the facility for 4 weeks after shipment. Animals were housed at a specialized and qualified facility (Medizinisch-Experimentelles Zentrum (MEZ) Universitat Leipzig, Medizinische Fakultat). Animals were identified by earmarking.

All mice were immunized by intradermal injection of 100 μl Collagen-CFA-Emulsion on day 0 (CFA stands for complete Freund's adjuvant). On day 21 CIA was boosted by injecting 100 μl of Collagen-IFA-Emulsion (IFA stands for incomplete Freund's adjuvant).

When an animal exceeded a score threshold of 5, CIA was considered to be established. The pharmacological treatment was started on that day and was performed for different time intervals. Eight mice obtained 100 μl i.v. of WF10 in a dosage of 0.25 ml/kg bodyweight on day 1, 3 and 5 from the onset of CIA. Eleven mice obtained 100 μl i.v. of S_PC-3-LipoWF10 providing a WF10 dosage of approximately 0.25 ml/kg bodyweight and twelve mice obtained 100 μl i.v. of S_PC-3-LipoWF10 (1:10 diluted) providing a WF10 dosage of approximately 0.025 ml/kg bodyweight. The control group comprised 10 mice, which received 200 μl NaCl. All dosing was performed on day 1, 3 and 5 of the onset of CIA. The liposome preparation “S_PC-3-LipoWF10” contained WF10 as the inner phase and saline as the outer phase and hydrogenated soybean phospholipid as the lipid (see Example 9). The liposomes had a WF10 inclusion rate of 5% which provides an approximate volume of 5 μL of WF10 in the 100 μL preparation. The S_PC-3-LipoWF10 1:10 dilution was a dilution of the original liposome preparation by a factor of 10, therefore, as a result, the absolute amount of WF10 was reduced although it is expected that the liposomal vesicles will continue to contain essentially pure WF10. Animals which developed CIA within the first 35 days after the immunization were included into the study. Animals which developed CIA after this time point were excluded.

Starting on day 17 measured from the initial collagen injection all mice were scored daily. Persons who evaluated the animals' score were blind with respect to the pharmacological treatment. All limbs were inspected for redness and swelling. The maximum score per limb was 15/day and the maximum score for one animal was a total 60/day. From these single values the mean value was calculated for each group.

### Results

Starting on day 17 after the first immunization the animals were scored for signs of collagen induced arthritis (CIA). When the score for an animal exceeded a value of 5, the CIA was considered to be induced and this day was termed dl for this animal. Similarly, the n^{th} day after onset of CIA was denoted do for each animal. FIG. 21 shows the mean values of the CIA score during the experiment from d0 (one day before the onset of CIA) to d22.

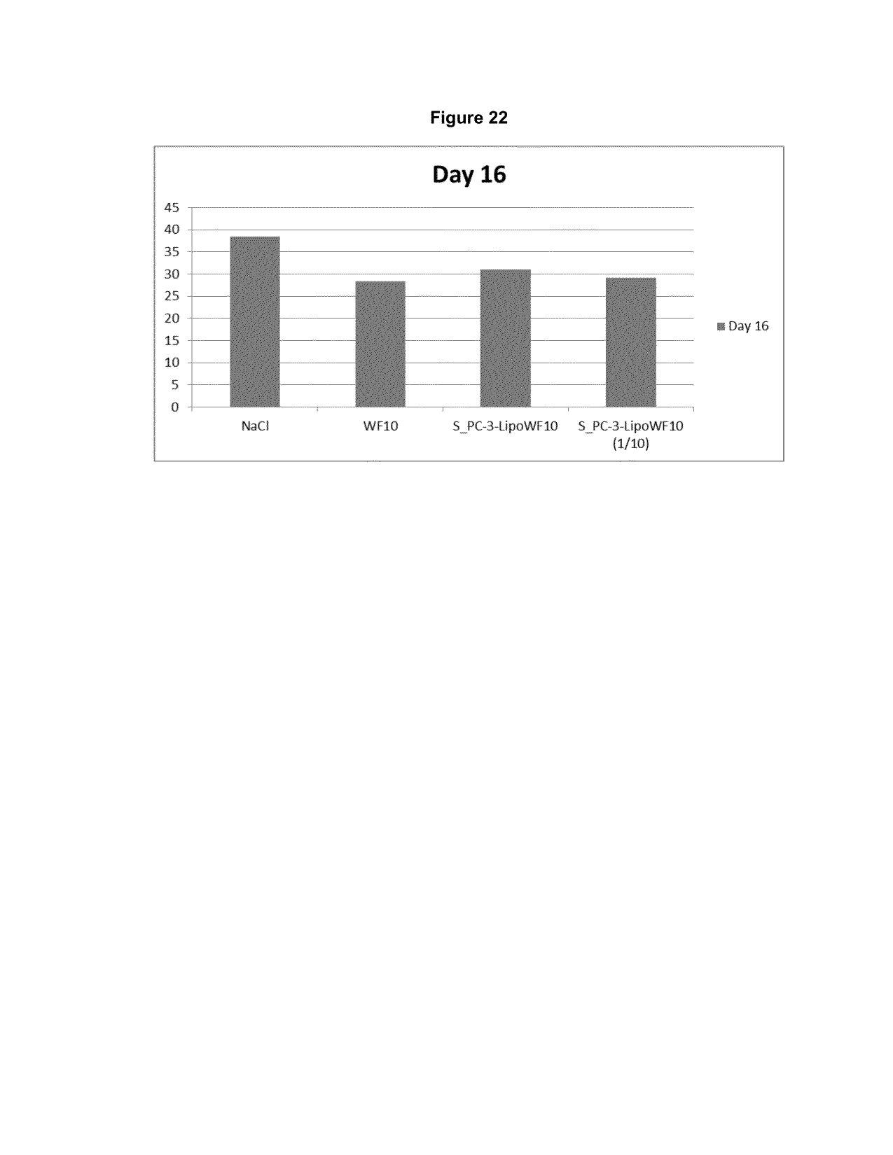

In CIA experiments, the effect of treatment usually starts around d12 and the separation is strongest around d15-d17. After that, the disease-score goes down due to the model. FIG. 22 shows the mean values scores for d16.

The relevant portions of all publications, patents and patent applications are herein incorporated by reference to the same extent as if each individual publication, patent or patent application was specifically and individually indicated to be incorporated by reference. Where a term in the present application is found to be defined differently in a document incorporated herein by reference, the definition provided herein is to serve as the definition for the term.

TABLE 1

| Abbreviation | CAS¹ | Name | Type |
|---|---|---|---|
| DDPC | 3436-44-0 | 1,2-Didecanoyl-sn-glycero-3- | Phosphatidylcholine |
| | | phosphocholine | |
| DEPA-NA | 80724- | 1,2-Dierucoyl-sn-glycero-3- | Phosphatidic acid |
| | 31-8 | phosphate (Sodium Salt) | |
| DEPC | 56649- | 1,2-Dierucoyl-sn-glycero-3- | Phosphatidylcholine |
| | 39-9 | phosphocholine | |
| DEPE | 988-07-2 | 1,2-Dierucoyl-sn-glycero-3- | Phosphatidylethanolamine |
| | | phosphoethanolamine | |
| DEPG-NA | | 1,2-Dierucoyl-sn-glycero- | Phosphatidylglycerol |
| | | 3[Phospho-rac-(1-glycerol . . . ) | |
| | | (Sodium Salt) | |
| DLOPC | 998-06-1 | 1,2-Dilinoleoyl-sn-glycero-3- | Phosphatidylcholine |
| | | phosphocholine | |
| DLPA-NA | | 1,2-Dilauroyl-sn-glycero-3- | Phosphatidic acid |
| | | phosphate (Sodium Salt) | |
| DLPC | 18194- | 1,2-Dilauroyl-sn-glycero-3- | Phosphatidylcholine |
| | 25-7 | phosphocholine | |
| DLPE | | 1,2-Dilauroyl-sn-glycero-3- | Phosphatidylethanolamine |
| | | phosphoethanolamine | |
| DLPG-NA | | 1,2-Dilauroyl-sn-glycero- | Phosphatidylglycerol |
| | | 3[Phospho-rac-(1-glycerol . . . ) | |
| | | (Sodium Salt) | |
| DLPG-NH4 | | 1,2-Dilauroyl-sn-glycero- | Phosphatidylglycerol |
| | | 3[Phospho-rac-(1-glycerol . . . ) | |
| | | (Ammonium Salt) | |
| DLPS-NA | | 1,2-Dilauroyl-sn-glycero-3- | Phosphatidylserine |
| | | phosphoserine (Sodium Salt) | |
| DMPA-NA | 80724-3 | 1,2-Dimyristoyl-sn-glycero-3- | Phosphatidic acid |
| | | phosphate (Sodium Salt) | |
| DMPC | 18194- | 1,2-Dimyristoyl-sn-glycero-3- | Phosphatidylcholine |
| | 24-6 | phosphocholine | |
| DMPE | 988-07-2 | 1,2-Dimyristoyl-sn-glycero-3- | Phosphatidylethanolamine |
| | | phosphoethanolamine | |
| DMPG-NA | 67232- | 1,2-Dimyristoyl-sn-glycero- | Phosphatidylglycerol |
| | 80-8 | 3[Phospho-rac-(1-glycerol . . . ) | |
| | | (Sodium Salt) | |
| DMPG-NH4 | | 1,2-Dimyristoyl-sn-glycero- | Phosphatidylglycerol |
| | | 3[Phospho-rac-(1-glycerol . . . ) | |
| | | (Ammonium Salt) | |
| DSPG-NH4 | 108347- | 1,2-Distearoyl-sn-glycero- | Phosphatidylglycerol |
| | 80-4 | 3[Phospho-rac-(1-glycerol . . . ) | |
| | | (Ammonium Salt) | |
| DMPG- | | 1,2-Dimyristoyl-sn-glycero- | Phosphatidylglycerol |
| NH4/NA | | 3[Phospho-rac-(1-glycerol . . . ) | |
| | | (Sodium/Ammonium Salt) | |
| DMPS-NA | | 1,2-Dimyristoyl-sn-glycero-3- | Phosphatidylserine |
| | | phosphoserine (Sodium Salt) | |
| DOPA-NA | | 1,2-Dioleoyl-sn-glycero-3- | Phosphatidic acid |
| | | phosphate (Sodium Salt) | |
| DOPC | 4235- | 1,2-Dioleoyl-sn-glycero-3- | Phosphatidylcholine |
| | 95-4 | phosphocholine | |
| DOPE | 4004-5- | 1,2-Dioleoyl-sn-glycero-3- | Phosphatidylethanolamine |
| | 1- | phosphoethanolamine | |
| DOPG-NA | 62700- | 1,2-Dioleoyl-sn-glycero- | Phosphatidylglycerol |
| | 69-0 | 3[Phospho-rac-(1-glycerol . . . ) | |
| | | (Sodium Salt) | |
| DOPS-NA | 70614- | 1,2-Dioleoyl-sn-glycero-3- | Phosphatidylserine |
| | 14-1 | phosphoserine (Sodium Salt) | |
| DPPA-NA | 71065- | 1,2-Dipalmitoyl-sn-glycero-3- | Phosphatidic acid |
| | 87-7 | phosphate (Sodium Salt) | |
| DPPC | 63-89-8 | 1,2-Dipalmitoyl-sn-glycero-3- | Phosphatidylcholine |
| | | phosphocholine | |
| DPPE | 923-61- | 1,2-Dipalmitoyl-sn-glycero-3- | Phosphatidylethanolamine |
| | 5 | phosphoethanolamine | |
| DPPG-NA | 67232- | 1,2-Dipalmitoyl-sn-glycero- | Phosphatidylglycerol |
| | 81-9 | 3[Phospho-rac-(1-glycerol . . . ) | |
| | | (Sodium Salt) | |
| DPPG-NH4 | 73548- | 1,2-Dipalmitoyl-sn-glycero- | Phosphatidylglycerol |
| | 70-6 | 3[Phospho-rac-(1-glycerol . . . ) | |
| | | (Ammonium Salt) | |
| DPPS-NA | | 1,2-Dipalmitoyl-sn-glycero-3- | Phosphatidylserine |
| | | phosphoserine (Sodium Salt) | |
| DSPA-NA | 108321- | 1,2-Distearoyl-sn-glycero-3- | Phosphatidic add |
| | 18-2 | phosphate (Sodium Salt) | |
| DSPC | 816-94- | 1,2-Distearoyl-sn-glycero-3- | Phosphatidylcholine |
| | 4 | phosphocholine | |
| DSPE | 1069- | 1,2-Distearoyl-sn-glycero-3- | Phosphatidylethanolamine |
| | 79-0 | phosphoethanolamine | |
| DSPG-NA | 67232- | 1,2-Distearoyl-sn-glycero- | Phosphatidylglycerol |
| | 82-0 | 3[Phospho-rac-(1-glycerol . . . ) | |
| | | (Sodium Salt) | |
| DSPS-NA | | 1,2-Distearoyl-sn-glycero-3- | Phosphatidylserine |
| | | phosphoserine (Sodium Salt) | |
| Egg | | | |
| Sphingomyelin | | | |
| empty Liposome | | | |
| EPC | | Egg-PC | Phosphatidylcholine |
| HEPC | | Hydrogenated Egg PC | Phosphatidylcholine |
| HSPC | | High purity Hydrogenated | Phosphatidylcholine |
| | | Soy PC | |
| HSPC | | Hydrogenated Soy PC | Phosphatidylcholine |
| LYSOPC | 18194- | 1-Myristoyl-sn-glycero-3- | Lysophosphatidylcholine |
| MYRISTIC | 24-6 | phosphocholine | |
| LYSOPC | 17364- | 1-Palmitoyl-sn-glycero-3- | Lysophosphatidylcholine |
| PALMITIC | 16-8 | phosphocholine | |
| LYSOPC | 19420- | 1-Stearoyl-sn-glycero-3- | Lysophosphatidylcholine |
| STEARIC | 57-6 | phosphocholine | |
| Milk | | 1-Myristoyl-2-palmitoyl-sn- | Phosphatidylcholine |
| Sphingomyelin | | glycero 3-phosphocholine | |
| MPPC | | | Phosphatidylcholine |
| MSPC | | 1-Myristoyl-2-stearoyl-sn- | Phosphatidylcholine |
| | | glycero-3-phosphocholine | |
| PMPC | | 1-Palmitoyl-2-myristoyl-sn- | Phosphatidylcholine |
| | | glycero-3-phosphocholine | |
| POPC | 26853- | 1-Palmitoyl-2-oleoyl-sn- | Phosphatidylcholine |
| | 31-6 | glycero-3-phosphocholine | |
| POPE | | 1-Palmitoyl-2-oleoyl-sn- | Phosphatidylethanolamine |
| | | glycero-3- | |
| | | phosphoethanolamine | |
| POPG-NA | 81490- | 1-Palmitoyl-2-oleoyl-sn- | Phosphatidylglycerol |
| | 05-3 | glycero-3[Phospho-rac-(1- | |
| | | glycerol) . . . ] (Sodium Salt) | |
| PSPC | | 1-Palmitoyl-2-stearoyl-sn- | Phosphatidylcholine |
| | | glycero-3-phosphocholine | |
| SMPC | | 1-Stearoyl-2-myristoyl-sn- | Phosphatidylcholine |
| | | glycero-3-phosphocholine | |
| SOPC | | 1-Stearoyl-2-oleoyl-sn- | Phosphatidylcholine |
| | | glycero-3-phosphocholine | |
| SPPC | | 1-Stearoyl-2-palmitoyl-sn- | Phosphatidylcholine |
| | | glycero-3-phosphocholine | |

¹Chemical Abstracts Service Registry Number

TABLE 2
WF10 Liposomes prepared from DPPC, DPPC/DMPC, hydrogenated
soybean phospholipid (3_PC3) and 3_PC3/DMPC

| | | | | | Leaking at |
|---|---|---|---|---|---|
| | Amount | Amount | Extrusion | Tight at | or above |
| Preparation | Lipid | WF10 | Temp [° C.] | [° C.] | [° C.] |
| DPPC | 394 mg | 13500 μL | 45° C. | 38 | 41 |
| DPPC/DMPC | 143 mg/ | 5400 μL | 45° C. | 22 | 38 |
| 9:1 | 15 mg | | | | |
| S_PC3 | 78 mg | 2500 μL | 60° C. | 50 | 60 |
| S_PC3/DMPC | 82 mg/ | 4184 μL | 60° C. | | |
| 3:5 | 42 mg | | | | |
| S_PC3/DMPC | 25 mg/ | 2500 μL | 60° C. | | |
| 5:3 | 42 mg | | | | |

TABLE 3
Analytical characterization of the manufactured
solutions from Example 15

| Sample | Osmolality* | |
|---|---|---|
| No. | [mosmol/kg] | Concentration of Active |
| 1 | 305 (303; 307) | |
| 2 | 301 (300; 302) | Chlorite: 4773 ppm (by UV) (1.12-fold |
| | | compared to WF10 {4250 ppm}) |
| 3 | 300.5 (299; 302) | Chlorate: 13 844 ppm (calculated) (9.23-fold |
| | | compared to WF10 {1500 ppm}) |
| 4 | 304 (303; 303; 306) | Chlorite: 5887 ppm (by UV) (1.39-fold |
| | | compared to WF10 {4250 ppm}) |

TABLE 4
Absorption Measurements From Example 15

| Sample | Initial | Fridge | Room | Forced |
|---|---|---|---|---|
| No. | test | storage | storage | leakage |
| 5 | <LOD | <LOD | <LOD | 3.7623 |
| 6 | <LOD | <LOD | 0.1816 | 3.7970 |
| 7 | <LOD | <LOD | <LOD | 3.1831 |
| 8 | <LOD | 0.1243 | 1.1388 | 3.8322 |
| 9 | <LOD | <LOD | <LOD | 3.6707 |
| 10 | <LOD | <LOD | 0.4243 | 3.7630 |
| 11 | <LOD | <LOD | <LOD | 3.3011 |
| 12 | <LOD | <LOD | 1.0170 | 3.8020 |
| 13 | <LOD | <LOD | <LOD | 3.5692 |
| 14 | <LOD | <LOD | <LOD | 3.5893 |
| 15 | <LOD | <LOD | <LOD | 3.5574 |
| 16 | <LOD | <LOD | 0.2286 | 3.7784 |
| 17 | <LOD | <LOD | <LOD | 3.8766 |

TABLE 5

| | | | DPPC | Chlorite | Chlorite | Chlorite |
|---|---|---|---|---|---|---|
| Lipid | Liposome | | recovery | encapsulation | recovery | recovery |
| formulation | size (nm) | PdI | (%) | rate (%) | (encapsulated) % | (encapsulated)* % |
| DPPC (Int) | 308.8 | 0.375 | | | | |
| DPPC (Ret) | 5042 | 1.000 | 83.0 | 93.6 | 6.4 | 7.7 |
| DPPC/DMPC | 111.1 | 0.314 | | | | |
| (Int) | | | | | | |
| DPPC/DMPC | 117.4 | 0.381 | 76.1 | 92.3 | 4.4 | 5.8 |
| (Ret) | | | | | | |
| DPPC/DPPG | 145.8 | 0.303 | | | | |
| (Int) | | | | | | |
| DPPC/DPPG | 141.6 | 0.295 | 65.3 | 97.6 | 2.5 | 3.9 |
| (Ret) | | | | | | |
| DPPC/DMPG | 128.9 | 0.306 | | | | |
| (Int) | | | | | | |
| DPPC/DMPG | 129.0 | 0.277 | 73.7 | 97.0 | 2.5 | 3.5 |
| (Ret) | | | | | | |

Int = intermediate product
Ret = retentate (after ultra/diafiltration)
*Theoretical recovery of the encapsulated chlorite assuming 100% DPPC recovery

TABLE 6

| | | | DPPC | Chlorite | Chlorite | Chlorite |
|---|---|---|---|---|---|---|
| Lipid | Liposome | | recovery | encapsulation | recovery | recovery |
| formulation | size (nm) | PdI | (%) | rate (%) | (encapsulated) % | (encapsulated)* % |
| DPPC (Int) | 125.2 | 0.283 | | | | |
| DPPC (Ret) | 309.3 | 0.466 | 74.0 | 94.8 | 8.8 | 11.9 |
| DPPC/DMPC | 127.8 | 0.341 | | | | |
| (Int) | | | | | | |
| DPPC/DMPC | 123.8 | 0.346 | 75.5 | 90.5 | 4.1 | 5.4 |
| (Ret) | | | | | | |
| DPPC/DPPG | — | — | | | | |
| (Int) | | | | | | |
| DPPC/DPPG | 147.5 | 0.258 | 73.1 | 95.8 | 2.1 | 2.9 |
| (Ret) | | | | | | |
| DPPC/DMPG | 316.6 | 0.362 | | | | |
| (Int) | | | | | | |
| DPPC/DMPG | 327.3 | 0.520 | 56.9 | 82.3 | 2.5 | 4.4 |
| (Ret) | | | | | | |

Int = intermediate product
Ret = retentate (after ultra/diafiltration)
*Theoretical recovery of the encapsulated chlorite assuming 100% DPPC recovery

TABLE 7

| | | | DPPC | Chlorite | Chlorite | Chlorite |
|---|---|---|---|---|---|---|
| Lipid | Liposome | | recovery | encapsulation | recovery | recovery |
| formulation | size (nm) | PdI | (%) | rate (%) | (encapsulated) % | (encapsulated)* % |
| DPPC (Int) | 124.2 | 0.317 | | | | |
| DPPC (Ret) | 117.7 | 0.285 | 66.8 | 91.6 | 5.6 | 8.4 |
| DPPC/DMPC | 118.2 | 0.283 | | | | |
| (Int) | | | | | | |
| DPPC/DMPC | 118.5 | 0.319 | 70.7 | 53.0 | 0.7 | 1.0 |
| (Ret) | | | | | | |
| DPPC/DPPG | 133.2 | 0.239 | | | | |
| (Int) | | | | | | |
| DPPC/DPPG | 132.3 | 0.236 | 70.0 | 74.1 | 1.6 | 2.3 |
| (Ret) | | | | | | |
| DPPC/DMPG | 123.5 | 0.310 | | | | |
| (Int) | | | | | | |
| DPPC/DMPG | 145.2 | 0.241 | 71.3 | 39.4 | 0.6 | 0.9 |
| (Ret) | | | | | | |

Int = intermediate product
Ret = retentate (after ultra/diafiltration)
*Theoretical recovery of the encapsulated chlorite assuming 100% DPPC recovery

TABLE 8

| | | Absorption | Duration of | Duration of |
|---|---|---|---|---|
| Concentration | | in o-tolidin | storage | storage |
| of WF10^{a} | Lipid(s) | test | (days) | (weeks) |
| WF10 (1x) | DPPC | <LOD | 65 | 9 |
| WF10 (1x) | DPPC, DMPC | <LOD^{b} | 92 | 13 |
| WF10 (1x) | DPPC, DPPG | <LOD | 86 | 12 |
| WF10 (1x) | DPPC, DMPG | <LOD | 86 | 12 |
| WF10 (2x) | DPPC | 0.170599997 | 71 | 10 |
| WF10 (2x) | DPPC, DMPC | 0.226199995 | 71 | 10 |
| WF10 (2x) | DPPC, DPPG | <LOD | 70 | 10 |
| WF10 (2x) | DPPC, DMPG | 0.1307000071 | 70 | 10 |

^{a}WF10 (1x) indicates that the liposomes had contained WF10 in its normal concentration. This avoids a significant difference in osmotic pressure relative to the saline as the outer phase. WF10 (2x) indicates that the liposomes had contained WF10 in a double concentration causing osmotic stress relative to the saline of the outer phase.
^{b}LOD had been established as an absorption value of 0.095

1. A liposomal composition comprising liposomes having at least one lipid bilayer and chlorite, chlorate or a mixture thereof encapsulated inside the liposomes, wherein the lipid bilayer is comprised of one or more suitable lipids.

2. The composition of claim 1, wherein the composition comprises chlorite, and wherein the chlorite is a stabilized chlorite.

3. The composition of claim 2, wherein the composition comprises chlorite and wherein the chlorite is OXO-K993.

4. The composition of claim 2, wherein the stabilized chlorite comprises 1-10%, 10-20%, 20-30%, 30-50% or 50-90% (w/v) of OXO-K993.

5. The composition of claim 1, wherein the composition comprises, based on the total encapsulated ion content of the composition, about 0.01% (w/w) to about 50% (w/w), about 0.1% (w/w) to about 20% (w/w), about 0.5% (w/w) to about 10% (w/w), about 0.3% (w/w) to about 3% (w/w) or about 1.0% (w/w) of encapsulated chlorite.

6. The composition of claim 1, wherein the composition comprises, based on the total encapsulated ion content of the composition, about 0.01% (w/w) to about 50% (w/w), about 0.1% (w/w) to about 20% (w/w), about 0.5% (w/w) to about 10% (w/w), about 0.3% (w/w) to about 3% (w/w) or about 1.0% of encapsulated chlorate.

7. The composition of claim 1, wherein the composition comprises, based on the total encapsulated ion content of the composition, about 0.01% (w/w) to about 50% (w/w), about 0.1% (w/w) to about 20% (w/w), about 0.5% (w/w) to about 10% (w/w), about 0.3% (w/w) to about 3% (w/w) or about 1.0% of a mixture of encapsulated chlorite and chlorate.

8. The composition of claim 1, wherein the encapsulated chlorite, chlorate or mixture thereof has a pH that is about 5 to about 14, about 6 to about 13, about 8 to about 12.5, or about 10 to about 12.

9. The composition of claim 1, wherein the lipids comprised in the liposomes are suitable for the entrapment of chlorite, chlorate or a mixture thereof having a pH of about 5 to about 14, about 6 to about 13, about 8 to about 12.5, or about 10 to about 12.

10. The composition of claim 1, wherein the suitable lipids are selected from the group consisting of a phospholipid, a sphingolipid, and mixtures thereof.

11. The composition of claim 10, wherein the phospholipid is a phosphatidylcholine (PC) comprising saturated or unsaturated fatty acyl chains of sufficient length, and the sphingolipid is sphingomyelin (SM).

12. The composition of claim 1, wherein the suitable lipids are selected from those that form liposomes that remain stable for an acceptable period of time at storage temperatures falling within the range of about 5° C. to about 50° C.

13. The composition of claim 12, wherein the suitable lipids are selected from those that form liposomes that are impermeable to leakage of ions at a temperature of about 5° C.

14. The composition of claim 1, wherein the liposomes comprise two or more types of suitable lipids.

15. The composition of claim 14, wherein the liposomes comprise two types of suitable lipids and the lipids are present in a molar ratio of 20:1 to 1:1, 15:1 to 5:1 or 10:1 to 9:1.

16. The composition of claim 1, further comprising at least one additional component that enhances the rigidity and/or reduce the permeability of the lipid bilayer(s).

17. The composition of claim 16, wherein the at least one additional component is present in an amount from about 0.1 to about 50%, about 1 to about 30%, about 5 to about 25% or about 10 to about 20% of the total lipid content.

18. The composition of claim 1, wherein the at least one additional component is selected from cholesterol and cholesterol sulfate.

19. The composition of claim 1, further comprising at least one additional component that provides the liposomes with a zeta potential that reduces aggregation of the liposomes.

20. The composition of claim 19, wherein the zeta potential that reduces aggregation of the liposomes is at least more positive than +5 mV or more negative than −5 mV.

21. The composition of claim 1, wherein the at least one additional component that provides the liposomes with a zeta potential that reduces aggregation of the liposomes is a charged lipid.

22. The composition of claim 21, wherein the charged lipid is a negatively charged lipid.

23. The composition of claim 22, wherein the negatively charged lipid is a phosphatidyl glycerol, a phosphatidyl ethanolamine, a phosphatidyl serine or a phosphatidic acid.

24. The composition of claim 22, wherein the negatively charged lipid is selected from the salts of 1,2,-dipalmitoyl-sn-glycero-3-phosphoglycerol (DPPG), 1,2-dimyristoyl-sn-glycero-3-phosphoglycerol (DMPG), 1,2-dioleoyl-sn-glycero-3-phosphoglycerol (DOPG), 1,2-distearoyl-sn-glycero-3-phosphoglycerol (DSPG), 1,2-distearoyl-sn-glycero-3-phosphoethanolamine (DSPE), or 1,2-distearoyl-sn-glycero-3-phosphoethanolamine-methyl-polyethyleneglycol (MPEG-DSPE), and mixtures thereof.

25. The composition of claim 21, wherein the charged lipid is present in an amount that provides a ratio of uncharged:charged lipids of 20:1 to 1:1, 15:1 to 5:1 or 10:1 to 9:1.

26. The composition of claim 1, wherein the one or more suitable lipids are selected from 1,2-dipalmitoyl-2-sn-glycero-3-phosphocholine (DPPC), 1,2-dimyristoyl-2-sn-glycero-3-phosphocholine (DMPC), 1-myristoyl-2-stearoyl-sn-glycero-3-phosphocholine (MSPC), 1-palmitoyl-2-myristoyl-sn-glycero-3-phosphocholine (PMPC), 1,2-distearoyl-sn-glycero-3-phosphocholine (DSPC), hydrogenated Egg PC (HEPC), 1-palmitoyl-2-stearoyl-sn-glycero-3-phosphocholine (PSPC), 1-stearoyl-2-myristoyl-sn-glycero-3-phosphocholine (SMPC), 1-stearoyl-2-oleoyl-sn-glycero-3-phosphocholine (SOPC), 1-stearoyl-2-palmitoyl-sn-glycero-3-phosphocholine (SPPC), diarachidoylphosphatidylcholine (DAPC), 1,2-dibehenoyl-sn-glycero-3-phosphocholine (DBPC), 1,2-dierucoyl-sn-glycero-3-phosphocholine (DEPC), 1,2-Dioleoyl-sn-glycero-3-phosphocholine (DOPC), hydrogenated soybean phospholipids, egg yolk phospholipids, sphingomyelin (SM), milk sphingomyelin and mixtures thereof.

27. The composition of claim 1, wherein the one or more suitable lipids are selected from 1,2-dipalmitoyl-2-sn-glycero-3-phosphocholine (DPPC), 1,2-dimyristoyl-2-sn-glycero-3-phosphocholine (DMPC), hydrogenated soybean phospholipids, egg yolk phospholipids, and mixtures thereof,

28. The composition of claim 1, wherein the one or more suitable lipids are 1,2-dipalmitoyl-2-sn-glycero-3-phosphocholine (DPPC) and 1,2-dimyristoyl-2-sn-glycero-3-phosphocholine (DMPC) in a molar ratio of 9:1; 1,2-dipalmitoyl-2-sn-glycero-3-phosphocholine (DPPC) and 1,2-dimyristoyl-sn-glycero-3-phosphoglycerol (DMPG) in a molar ratio of 9:1; 1,2-dipalmitoyl-2-sn-glycero-3-phosphocholine (DPPC) and 1,2,-dipalmitoyl-sn-glycero-3-phosphoglycerol (DPPG) in a molar ratio of 9:1 or DPPC.

29. The composition of claim 1, wherein the lipid bilayer is surface modified with molecules that increase hydrophilicity.

30. The composition of claim 1, wherein the lipid bilayer is modified by attaching cell-specific targeting moieties to its surface to facilitate association with a specific cell or tissue type.

31. A liposome comprising at least one lipid bilayer and chlorite, chlorate or a mixture thereof entrapped inside the liposome, wherein the lipid bilayer is comprised of one or more suitable lipids.

32. The liposome of claim 31, having an average diameter of about 80 nm to about 300 nm, about 90 nm to about 200 nm, about 100 nm to about 140 nm, about 80 nm to about 15 microns, about 300 nm to about 12 microns or about 7 microns to about 10 microns.

33. The liposome of claim 32, wherein the chlorite, chlorate or mixture thereof is entrapped with an efficiency or inclusion rate of 1% to about 50%, about 2% to about 25% or about 5% to about 15%.

34. A method of preparing liposomes having at least one lipid bilayer and chlorite, chlorate or a mixture thereof encapsulated inside the liposomes, wherein the lipid bilayer is comprised of one or more suitable lipids, the method comprising:
(a) adding an aqueous solution of chlorite, chlorate or a mixture thereof to a vessel having a film of the one or more lipids on at least a portion of an inner surface;
(b) agitating the vessel under conditions sufficient to remove wholly or partially the film from the inner surface to provide a turbid solution comprising the chlorite- and/or chlorate-entrapped liposomes;
(c) treating the turbid solution to reduce the average diameter of the liposomes to a desired amount; and
(d) optionally treating the liposomes to remove chlorite, chlorate or the mixture thereof from a solution external to the liposomes.

35. The method of claim 34, wherein the molar ratio of the one or more suitable lipids to the chlorite, chlorate or a mixture thereof in (a) is about 0.01:1 to about 10000:1, about 0.1:1 to about 5000:1, about 0.5:1 to about 2500:1, about 1:1 to about 1000:1, or about 0.1:1 to about 100:1.

36. A method of preparing liposomes having at least one lipid bilayer and chlorite, chlorate or a mixture thereof encapsulated inside the liposomes, wherein the lipid bilayer is comprised of one or more suitable lipids, wherein the method is an ethanol injection method.

37. The method of claim 36, wherein the ethanol injection method is performed using a crossflow technique.

38. A pharmaceutical composition comprising the liposomal composition of any one of claim 1, admixed with at least one physiologically acceptable carrier or excipient.

39. A method for treating a disease, disorder or condition for which administration of chlorite, chlorate or a mixture thereof is beneficial comprising administering an effective amount of the liposomal composition of claim 1 to a subject in need thereof.

40. A method for regulating macrophage function comprising administering an effective amount of the liposomal composition of claim 1 to a subject in need thereof.

41. The method of claim 40, wherein regulating macrophage function results in treatment of diseases that produce symptoms of chronic inflammation as a result of an inappropriate immune response.

42. The method of claim 39, wherein the diseases, disorders or conditions for which administration of chlorite, chlorate or a mixture thereof is beneficial are selected from autoimmune diseases, diseases caused by inappropriate immune response, wound healing, radiation syndrome and exposure to environmental toxins.

43. The method of claim 42, wherein the diseases, disorders or conditions are selected from myasthenia gravis, systemic lupus erythematosus, serum disease, diabetes, rheumatoid arthritis, juvenile rheumatoid arthritis, rheumatic fever, Sjorgen syndrome, systemic sclerosis, spondylarthropathies, Lyme disease, sarcoidosis, autoimmune hemolysis, autoimmune hepatitis, autoimmune neutropenia, autoimmune polyglandular disease, autoimmune thyroid disease, multiple sclerosis, inflammatory bowel disease, colitis, Crohn's disease, chronic fatigue syndrome, chronic obstructive pulmonary disease (COPD), graft rejection, graft vs. host disease, allergic asthma, allergic rhinitis, atopic dermatitis, inappropriate response to tissue insult, hepatitis B, hepatitis C, chronic hepatitis, obstructive bronchitis, emphysema, neoplastic disorders (cancer), HIV infection, AIDS, neurodegenerative disease, AIDS-associated dementia, microbial infections and other viral infections.

44. The method of claim 43, wherein the disease, disorder or condition is selected from allergic asthma, allergic rhinitis, atopic dermatitis, neoplastic disorder, spinal cord pathology, HIV infection and AIDS.

45. The method of claim 44, wherein the neoplastic disorder is a cancer of the gastrointestinal tract, head, neck, breast or pancreas.

46. A method comprising providing the liposomal composition of claim 1 to a user and informing the user of certain safety or clinical effects.

47. A method comprising providing a liposomal composition of claim 1 to a user and informing the user that the liposomal composition is more stable, target specific, or therapeutically effective than a non-liposomal composition that provides, or would be expected to provide, a similar therapeutic effect.

48. The method of claim 47, wherein the user is informed by way of published material.

49. A liposomal composition comprising one or more inner core phases and an outer continuous phase, the inner core phases comprising chlorite, chlorate or a mixture thereof and being contained in a plurality of liposomal vesicles wherein the walls of the vesicles comprise at least one lipid bilayer and the outer continuous phase of the liposomal composition is substantially free of chlorite and chlorate.

50. The composition of claim 49, wherein the outer continuous phase comprises sodium chloride.

51. The composition of claim 49, wherein the outer continuous phase has a pH of about 5-8.

52. The composition of claim 49, wherein the inner core phase has a pH of about 8-13.

53. The composition of claim 49, having a pH difference between the inner core phase and outer continuous phase of about 1 to about 7.

54. The composition of claim 49, wherein the liposomal composition shows pharmaceutically-acceptable stability from about 3-48 months.

55. The composition of claim 54, wherein the pharmaceutically acceptable stability is achieved with storage of the composition at a temperature of about 5° C. to about 50° C.

56. The composition of claim 49, wherein the composition is substantially free of degradation products.
